(19) United States
(12) Patent Application Publication
(10) Pub. No.: US 2024/0025913 A1
(43) Pub. Date: Jan. 25, 2024

(54) TERTIARY CARBOXAMIDE COMPOUNDS

(71) Applicant: Vivace Therapeutics, Inc., San Mateo, CA (US)

(72) Inventors: Andrei W. KONRADI, Burlingame, CA (US); Tracy Tzu-Ling Tang LIN, Redwood City, CA (US)

(21) Appl. No.: 18/249,704

(22) PCT Filed: Oct. 19, 2021
(86) PCT No.: PCT/US2021/055668

§ 371 Date: Apr. 19, 2023

Related U.S. Application Data
(60) Provisional application No. 63/094,627, filed on Oct. 21, 2020.

(51) Int. Cl. C07D 487/10; C07D 205/04; C07D 295/192; C07D 207/14; C07D 211/58; C07D 211/26; C07D 211/56; C07D 207/09; C07D 401/12; C07C 233/65; C07C 255/00; A61P 35/00

(52) U.S. Cl. CPC C07D 487/10; C07D 205/04; C07D 295/192; C07D 207/14; C07D 211/58; C07D 211/26; C07D 211/56; C07D 207/09; C07D 401/12; C07C 233/65; C07C 255/00; A61P 35/00

(57) ABSTRACT

Provided herein are compounds and pharmaceutical compositions comprising said compounds that are useful for treating diseases. Specific diseases include those that are mediated by YAP/TAZ or those that are modulated by the interaction between YAP/TAZ and TEAD.

### CROSS-REFERENCE

This application claims benefit of U.S. Provisional Patent Application No. 63/094,627, filed on Oct. 21, 2020, which is incorporated herein by reference in its entirety.

### BACKGROUND OF THE DISCLOSURE

YAP and TAZ are transcriptional co-activators of the Hippo pathway network and regulate cell proliferation, migration, and apoptosis. Inhibition of the Hippo pathway promotes YAP/TAZ translocation to the nucleus, wherein YAP/TAZ interact with transcriptional enhancer associate domain (TEAD) transcription factors and coactivate the expression of target genes and promote cell proliferation. Hyperactivation of YAP and TAZ and/or mutations in one or more members of the Hippo pathway network have been implicated in numerous cancers. Described herein are inhibitors associated with one or more members of the Hippo pathway network, such as inhibitors of YAP/TAZ or inhibitors that modulate the interaction between YAP/TAZ and TEAD.

### SUMMARY OF THE DISCLOSURE

Provided herein are bicyclic compounds and pharmaceutical compositions comprising said compounds. In some embodiments, the subject compounds are useful for the treatment of diseases.

In one aspect, the present disclosure provides a compound of Formula (A), or a pharmaceutically acceptable salt or solvate thereof:

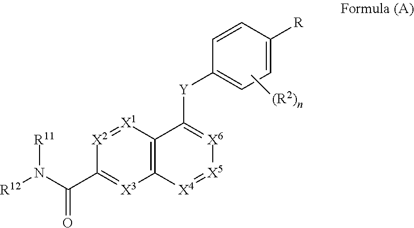

wherein,
X¹ is N or CR^{X1}; X² is N or CR^{X2}; X³ is N or CR^{X3}; X⁴ is N or CR^{X4}; X⁵ is N or CR^{X5}; X⁶ is N or CR^{X6};
Y is O, S, or NR³;
each R^{X1}, R^{X2}, R^{X3}, R^{X4}, R^{X5}, and R^{X6} is independently hydrogen, halogen, nitro, —OR³, —SR³, —CN, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₇cycloalkyl, or substituted or unsubstituted C₂-C₆heterocycloalkyl;
R is halogen, nitro, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, or substituted or unsubstituted C₁-C₆fluoroalkyl;
R¹¹ and R¹² on the same nitrogen atom taken together with the nitrogen atom to which they are attached to form:

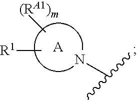

Ring A is substituted or unsubstituted N-containing heterocycloalkyl;
R¹ is hydrogen, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆ aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl;
m is 0, 1, 2, 3, 4, 5, or 6;
or each of R¹¹ and R¹² is independently —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R² is independently halogen, nitro, —CN, —OR³, —SR³, —S(═O)₂R³, —NR³R⁴, —C(═O)OR³, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R³ and R⁴ is independently hydrogen, —CN, substituted or unsubstituted C₁-C₆alkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or R³ and R⁴ on the same nitrogen atom taken together with the nitrogen atom to which they are attached to form a substituted or unsubstituted N-containing C₃-C₇ heterocycloalkyl; and
n is 0, 1, 2, 3, or 4.

In some embodiments, the compound of Formula (A) has the structure of Formula (I), or a pharmaceutically acceptable salt or solvate thereof:

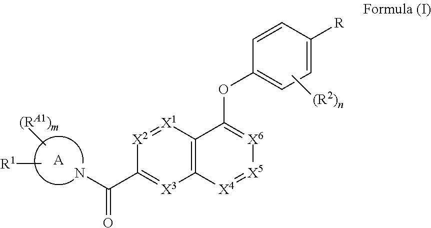

wherein,
Ring A is substituted or unsubstituted N-containing heterocycloalkyl;
X¹ is N or CR^{X1}; X² is N or CR^{X2}; X³ is N or CR^{X3}; X⁴ is N or CR^{X4}; X⁵ is N or CR^{X5}; X⁶ is N or CR^{X6};
each R^{X1}, R^{X2}, R^{X3}, R^{X4}, R^{X5}, and R^{X6} is independently hydrogen, halogen, nitro, —OR³, —SR³, —CN, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆ alkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₇cycloalkyl, or substituted or unsubstituted C₂-C₆heterocycloalkyl;
R is halogen, nitro, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, or substituted or unsubstituted C₁-C₆fluoroalkyl;
R¹ is hydrogen, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆ aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl;
each R² is independently halogen, nitro, —CN, —OR³, —SR³, —S(═O)₂R³, —NR³R⁴, —C(═O)OR³, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R³ and R⁴ is independently hydrogen, —CN, substituted or unsubstituted C₁-C₆alkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or R³ and R⁴ on the same nitrogen atom taken together with the nitrogen atom to which they are attached to form a substituted or unsubstituted N-containing C₃-C₇ heterocycloalkyl;
m is 0, 1, 2, 3, 4, 5, or 6; and
n is 0, 1, 2, 3, or 4.

Any combination of the groups described above for the various variables is contemplated herein. Throughout the specification, groups and substituents thereof are chosen by one skilled in the field to provide stable moieties and compounds.

In another aspect, the present disclosure provides a compound or pharmaceutically acceptable salt thereof, wherein the compound is a compound from Table 1, or a pharmaceutically acceptable salt or solvate thereof.

In another aspect, the present disclosure provides a pharmaceutical composition comprising a pharmaceutically acceptable excipient and a compound disclosed herein or a pharmaceutically acceptable salt or solvate thereof.

In another aspect, the present disclosure provides a method of inhibiting one or more of proteins encompassed by, or related to, the Hippo pathway in a subject, comprising administering to a subject a compound disclosed herein, or a pharmaceutically acceptable salt or solvate thereof.

In another aspect, the present disclosure provides a method of inhibiting transcriptional coactivator with PDZ binding motif/Yes-associated protein transcriptional coactivator (TAZ/YAP) in a subject comprising administering to a subject a compound disclosed herein, or a pharmaceutically acceptable salt or solvate thereof. In some embodiments, the subject has cancer, polycystic kidney disease, or liver fibrosis. In some embodiments, the cancer is selected from mesothelioma, hepatocellular carcinoma, meningioma, malignant peripheral nerve sheath tumor, Schwannoma, lung cancer, bladder carcinoma, cutaneous neurofibromas, prostate cancer, pancreatic cancer, glioblastoma, endometrial adenosquamous carcinoma, anaplastic thyroid carcinoma, gastric adenocarcinoma, esophageal adenocarcinoma, ovarian cancer, ovarian serous adenocarcinoma, melanoma, and breast cancer.

In another aspect, the present disclosure provides a method of treating cancer in a subject in need thereof comprising administering to the subject in need thereof a therapeutically effective amount of a compound disclosed herein, or a pharmaceutically acceptable salt or solvate thereof. In some embodiments, the cancer is selected from mesothelioma, hepatocellular carcinoma, meningioma, malignant peripheral nerve sheath tumor, Schwannoma, lung cancer, bladder carcinoma, cutaneous neurofibromas, prostate cancer, pancreatic cancer, glioblastoma, endometrial adenosquamous carcinoma, anaplastic thyroid carcinoma, gastric adenocarcinoma, esophageal adenocarcinoma, ovarian cancer, ovarian serous adenocarcinoma, melanoma, and breast cancer.

In another aspect, the present disclosure provides a method of treating polycystic kidney disease or liver fibrosis in a subject in need thereof comprising administering to the subject in need thereof a therapeutically effective amount of a compound disclosed herein, or a pharmaceutically acceptable salt or solvate thereof.

### DETAILED DESCRIPTION OF THE DISCLOSURE

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, in some embodiments, ranges and amounts are expressed as “about” a particular value or range. About also includes the exact amount. Hence “about 5 μL” means “about 5 μL” and also “5 μL.” Generally, the term “about” includes an amount that is expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly, or a hospice worker).

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each Ra is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each R^{f} is independently alkyl, fluoroalkyl, carbocyclyl, carbocyclylalkyl, aryl, aralkyl, heterocyclyl, heterocyclylalkyl, heteroaryl, or heteroarylalkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each Ra is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each R^{f} is independently alkyl, fluoroalkyl, carbocyclyl, carbocyclylalkyl, aryl, aralkyl, heterocyclyl, heterocyclylalkyl, heteroaryl, or heteroarylalkyl.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each Ra is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each R^{f} is independently alkyl, fluoroalkyl, carbocyclyl, carbocyclylalkyl, aryl, aralkyl, heterocyclyl, heterocyclylalkyl, heteroaryl, or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. In some embodiments, the points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each Ra is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each R^{f} is independently alkyl, fluoroalkyl, carbocyclyl, carbocyclylalkyl, aryl, aralkyl, heterocyclyl, heterocyclylalkyl, heteroaryl, or heteroarylalkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin, and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—CN, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2), and —R^{b}—S(O)ₜN(R^{a})₂ (where t is 1 or 2), where each Ra is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl, or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aryloxy” refers to a radical bonded through an oxygen atom of the formula —O-aryl, where aryl is as defined above.

“Aralkyl” refers to a radical of the formula —R^{c}-aryl where R is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, and in some embodiments, include fused, spiro, or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. In some embodiments, the carbocyclyl is saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₅ cycloalkyl). In other embodiments, a cycloalkyl comprises three to four carbon atoms (e.g., C₃-C₄ cycloalkyl). An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —CN, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(Ra)C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2), and —R^{b}—S(O)ₜN(R^{a})₂ (where t is 1 or 2), where each Ra is independently hydrogen, alkyl, fluoroalkyl, cycloalkyl, cycloalkylalkyl, aryl, aralkyl, heterocyclyl, heterocyclylalkyl, heteroaryl, or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Halo” or “halogen” refers to bromo, chloro, fluoro, or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” or “heterocycle” refers to a stable 3- to 18-membered non-aromatic ring radical that comprises two to twelve carbon atoms and from one to six heteroatoms selected from nitrogen, oxygen, and sulfur. Unless stated otherwise specifically in the specification, the heterocyclyl radical is a monocyclic, bicyclic, tricyclic, or tetracyclic ring system, which include fused, spiro, or bridged ring systems in some embodiments. The heteroatoms in the heterocyclyl radical are optionally oxidized. One or more nitrogen atoms, if present, are optionally quaternized. The heterocyclyl radical is partially or fully saturated. In some embodiments, the heterocyclyl is attached to the rest of the molecule through any atom of the ring(s). In some embodiments, the heterocyclyl is saturated, (i.e., containing single bonds only) or unsaturated (i.e., containing one or more double bonds or triple bonds.) A fully saturated heterocyclyl radical is also referred to as “heterocycloalkyl.” Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —CN, —R^{b}—CN, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2), and —R^{b}—S(O)ₜN(R^{a})₂ (where t is 1 or 2), where each Ra is independently hydrogen, alkyl, fluoroalkyl, cycloalkyl, cycloalkylalkyl, aryl, aralkyl, heterocyclyl, heterocyclylalkyl, heteroaryl, or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl. In some embodiments, the alkyl part of the heteroalkyl radical is optionally substituted as defined for an alkyl group.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen, and sulfur. As used herein, in some embodiments, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo-[4,5]thieno[2,3-d]pyrimidinyl, 6,7,8,9-tetrahydro-5H-cyclohepta[4,5]-thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2), and —R^{b}—S(O)ₜN(R^{a})₂ (where t is 1 or 2), where each Ra is independently hydrogen, alkyl, fluoroalkyl, cycloalkyl, cycloalkylalkyl, aryl, aralkyl, heterocyclyl, heterocyclylalkyl, heteroaryl, or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O— heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments, the compounds disclosed herein contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans). Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

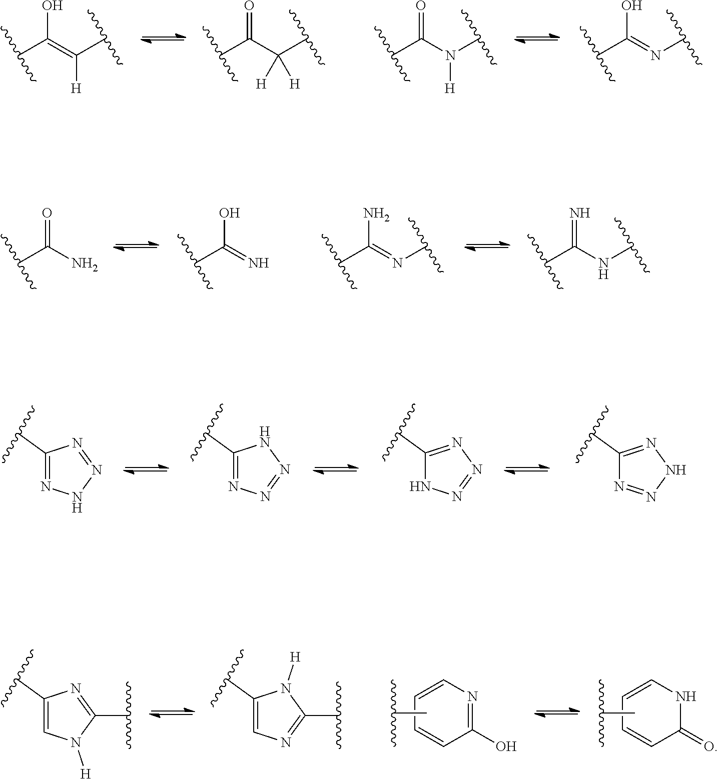

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. A pharmaceutically acceptable salt of any one of the compounds described herein is intended to encompass any and all pharmaceutically suitable salt forms. Pharmaceutically acceptable salts of the compounds described herein are optionally pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). In some embodiments, acid addition salts of basic compounds are prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines, and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins, and the like. See Berge et al., supra.

As used herein, “treatment” or “treating” or “palliating” or “ameliorating” are used interchangeably herein. These terms refer to an approach for obtaining beneficial or desired results including, but not limited to, therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is afflicted with the underlying disorder in some embodiments. For prophylactic benefit, in some embodiments, the compositions are administered to a patient at risk of developing a particular disease, or to a patient reporting one or more of the physiological symptoms of a disease, even though a diagnosis of this disease has not been made.

“Prodrug” is meant to indicate a compound that is converted under physiological conditions or by solvolysis to a biologically active compound described herein. Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. In some embodiments, prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian subject, cleaves to form a free hydroxy, free amino, or free mercapto group, respectively. Examples of prodrugs include, but are not limited to, acetate, formate, and benzoate derivatives of alcohol or amine functional groups in the active compounds and the like.

### Compounds

In some embodiments, the compounds disclosed herein are bicyclic compounds.

In one aspect, the present disclosure provides a compound of Formula (A), or a pharmaceutically acceptable salt or solvate thereof:

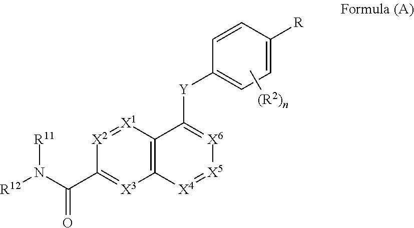

wherein,
X¹ is N or CR^{X1}; X² is N or CR^{X2}; X³ is N or CR^{X3}; X⁴ is N or CR^{X4}; X⁵ is N or CR^{X5}; X⁶ is N or CR^{X6};
Y is O, S, or NR³;
each R^{X1}, R^{X2}, R^{X3}, R^{X4}, R^{X5}, and R^{X6} is independently hydrogen, halogen, nitro, —OR³, —SR³, —CN, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₇cycloalkyl, or substituted or unsubstituted C₂-C₆heterocycloalkyl;
R is halogen, nitro, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, or substituted or unsubstituted C₁-C₆fluoroalkyl;
R¹¹ and R¹² on the same nitrogen atom taken together with the nitrogen atom to which they are
   attached to form:

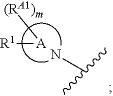

Ring A is substituted or unsubstituted N-containing heterocycloalkyl;
R¹ is hydrogen, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆ aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl;
m is 0, 1, 2, 3, 4, 5, or 6;
or each of R¹¹ and R¹² is independently —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
or R¹¹ is hydrogen; and R¹² is —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R² is independently halogen, nitro, —CN, —OR³, —SR³, —S(═O)₂R³, —NR³R⁴, —C(═O)OR³, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R³ and R⁴ is independently hydrogen, —CN, substituted or unsubstituted C₁-C₆alkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or R³ and R⁴ on the same nitrogen atom taken together with the nitrogen atom to which they are attached to form a substituted or unsubstituted N-containing C₃-C₇ heterocycloalkyl; and
n is 0, 1, 2, 3, or 4.

In some embodiments of a compound of Formula (A), or a pharmaceutically acceptable salt or solvate thereof, Y is S or NR³. In some embodiments of a compound of Formula (A), or a pharmaceutically acceptable salt or solvate thereof, Y is O; and R¹¹ and R¹² on the same nitrogen atom taken together with the nitrogen atom to which they are attached to form:

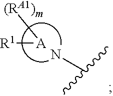

Ring A is substituted or unsubstituted N-containing heterocycloalkyl; or each of R¹¹ and R¹² is independently —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl.

In some embodiments, the compound of Formula (A) has the structure of Formula (I), or a pharmaceutically acceptable salt or solvate thereof:

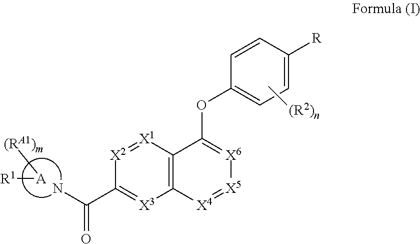

wherein,
Ring A is substituted or unsubstituted N-containing heterocycloalkyl;
X¹ is N or CR^{X1}; X² is N or CR^{X2}; X³ is N or CR^{X3}; X⁴ is N or CR^{X4}; X⁵ is N or CR^{X5}; X⁶ is N or CR^{X6};
each R^{X1}, R^{X2}, R^{X3}, R^{X4}, R^{X5}, and R^{X6} is independently hydrogen, halogen, nitro, —OR³, —SR³, —CN, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆ alkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₇cycloalkyl, or substituted or unsubstituted C₂-C₆heterocycloalkyl;
R is halogen, nitro, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, or substituted or unsubstituted C₁-C₆fluoroalkyl;
R¹ is hydrogen, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆ aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl;
each R² is independently halogen, nitro, —CN, —OR³, —SR³, —S(═O)₂R³, —NR³R⁴, —C(═O)OR³, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R³ and R⁴ is independently hydrogen, —CN, substituted or unsubstituted C₁-C₆alkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or R³ and R⁴ on the same nitrogen atom taken together with the nitrogen atom to which they are attached to form a substituted or unsubstituted N-containing C₃-C₇ heterocycloalkyl;
m is 0, 1, 2, 3, 4, 5, or 6; and
n is 0, 1, 2, 3, or 4.

In some embodiments of a compound of Formula (A) or Formula (I), or a pharmaceutically acceptable salt or solvate thereof, Ring A is substituted or unsubstituted monocyclic C₃-C₁₁heterocycloalkyl. In some embodiments, Ring A is C₃-C₉heterocycloalkyl comprising 1-3 N ring atoms, 0-1 O ring atoms, and 0-1 S ring atoms. In some embodiments, Ring A is C₃-C₁₁heterocycloalkyl comprising 1-2 N ring atoms, 0-1 O ring atoms, and 0-1 S ring atoms. In some embodiments, Ring A is C₃-C₉heterocycloalkyl comprising 1-2 N ring atoms and 0-1 O ring atoms. In some embodiments, Ring A is C₃-C₉heterocycloalkyl comprising 1-2 N ring atoms and 0-1 S ring atoms. In some embodiments, Ring A is C₃-C₇heterocycloalkyl comprising 1-2 N ring atoms and 0-1 O ring atoms. In some embodiments, Ring A is C₃-C₇heterocycloalkyl comprising 1-2 N ring atoms and 0-1 S ring atoms. In some embodiments, Ring A is C₃-C₇heterocycloalkyl comprising 1-2 N ring atoms. In some embodiments, Ring A is C₃-C₇heterocycloalkyl comprising 1 N ring atom.

In some embodiments of a compound of Formula (A) or Formula (I), or a pharmaceutically acceptable salt or solvate thereof, Ring A is substituted or unsubstituted monocyclic C₃-C₇heterocycloalkyl. In some embodiments,

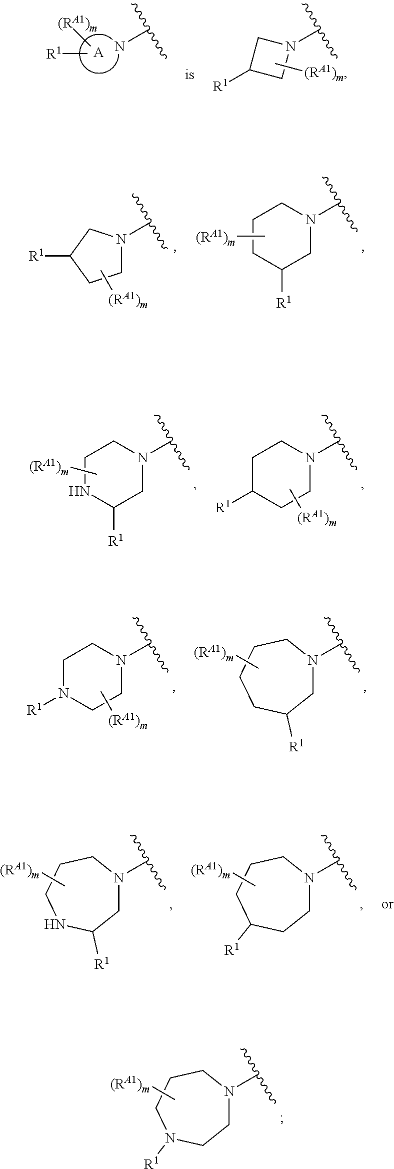

wherein each R^{A1} is independently selected from F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl; R³ is hydrogen, substituted or unsubstituted C₁-C₄alkyl, or substituted or unsubstituted C₃-C₆cycloalkyl; and m is 0, 1, 2, 3, 4, 5, or 6. In some embodiments,

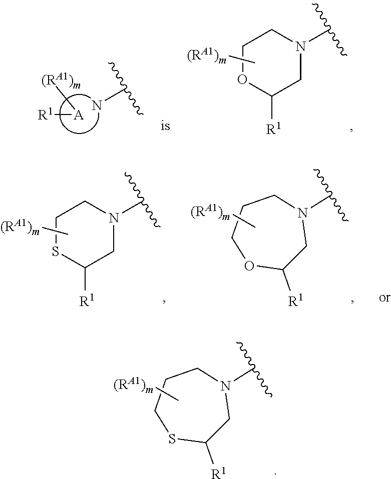

In some embodiments,

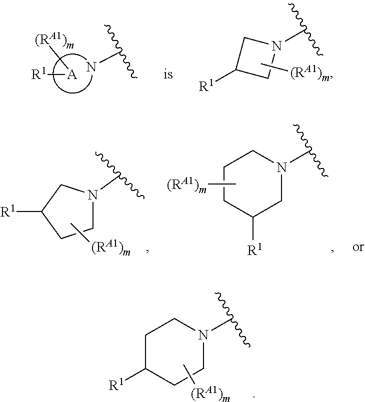

In some embodiments,

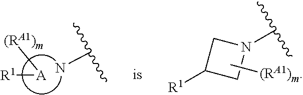

In some embodiments of a compound of Formula (A) or Formula (I), or a pharmaceutically acceptable salt or solvate thereof, Ring A is substituted or unsubstituted polycyclic C₃-C₁₆heterocycloalkyl.

In some embodiments, Ring A is substituted or unsubstituted fused- or spiro-C₄-C₁₂heterocycloalkyl. In some embodiments, Ring A is substituted or unsubstituted fused C₄-C₁₂heterocycloalkyl. In some embodiments, Ring A is substituted or unsubstituted spiro C₄-C₁₂heterocycloalkyl. In some embodiments of a compound of Formula (A) or Formula (I), or a pharmaceutically acceptable salt or solvate thereof, Ring A is substituted or unsubstituted fused C₄-C₁₂heterocycloalkyl. In some embodiments,

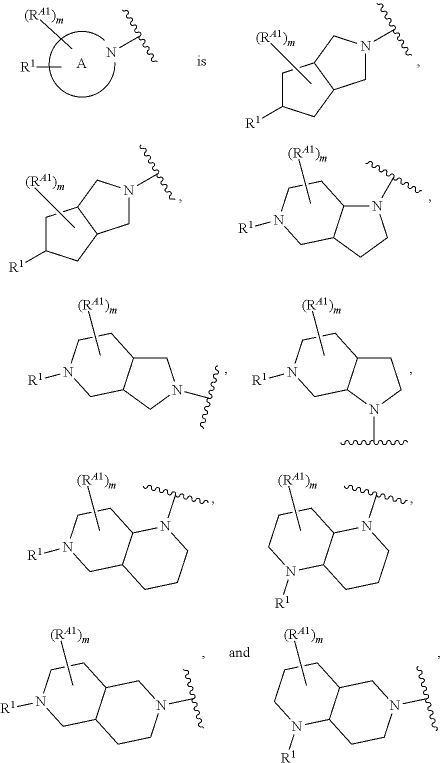

wherein each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl; R³ is hydrogen, substituted or unsubstituted C₁-C₄alkyl, or substituted or unsubstituted C₃-C₆cycloalkyl; and m is 0, 1, 2, 3, 4, 5, or 6.

In some embodiments of a compound of Formula (A) or Formula (I), or a pharmaceutically acceptable salt or solvate thereof, Ring A is substituted or unsubstituted spiro C₄-C₁₂heterocycloalkyl. In some embodiments,

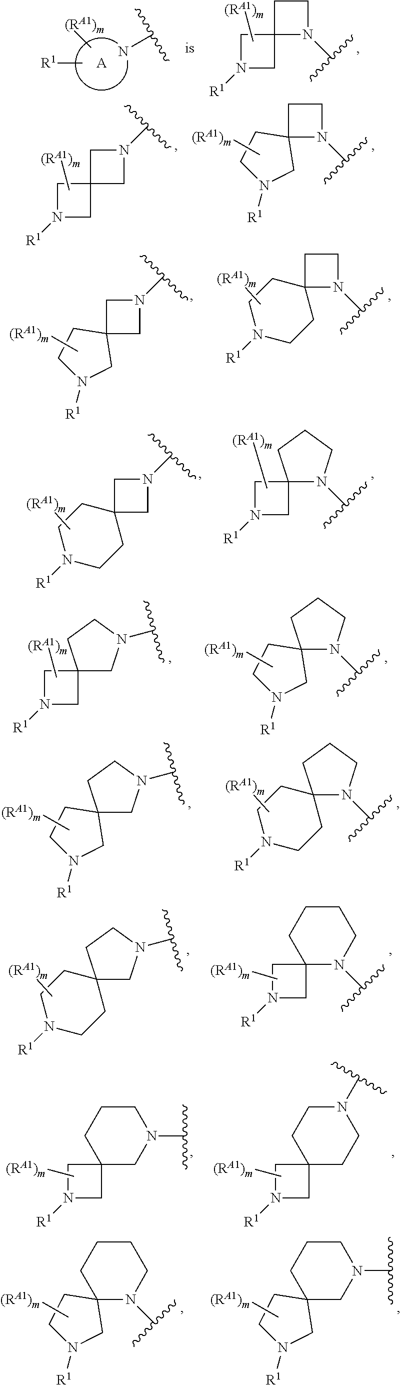

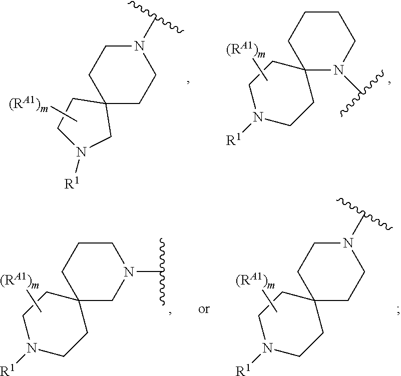

wherein each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl; R³ is hydrogen, substituted or unsubstituted C₁-C₄alkyl, or substituted or unsubstituted C₃-C₆cycloalkyl; and m is 0, 1, 2, 3, 4, 5, or 6.

In some embodiments,

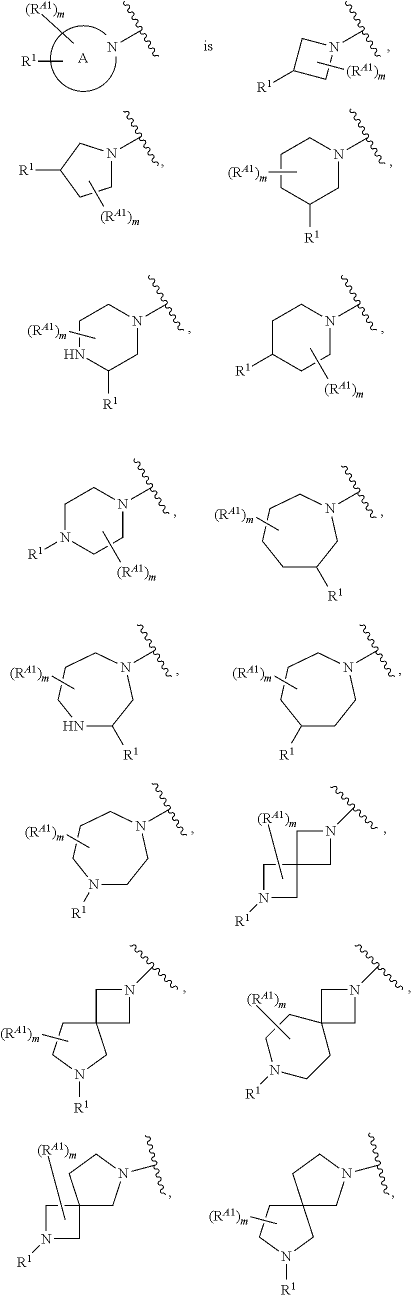

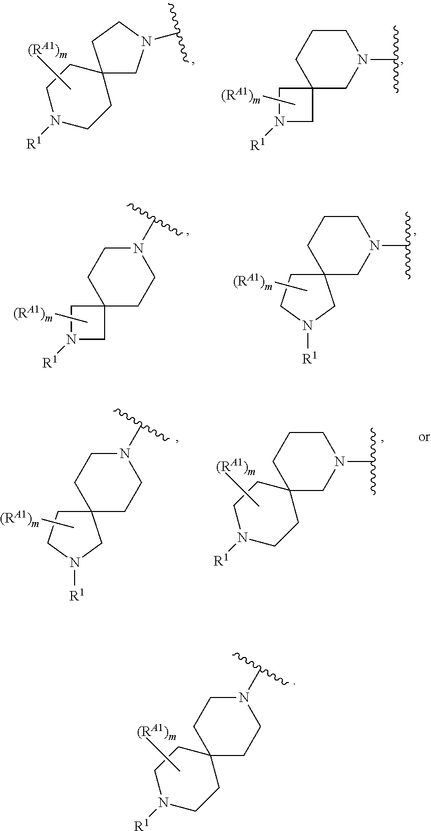

In some embodiments, m is 0, 1 or 2. In some embodiments, m is 0. In some embodiments, m is 1. In some embodiments, m is 2.

In some embodiments,

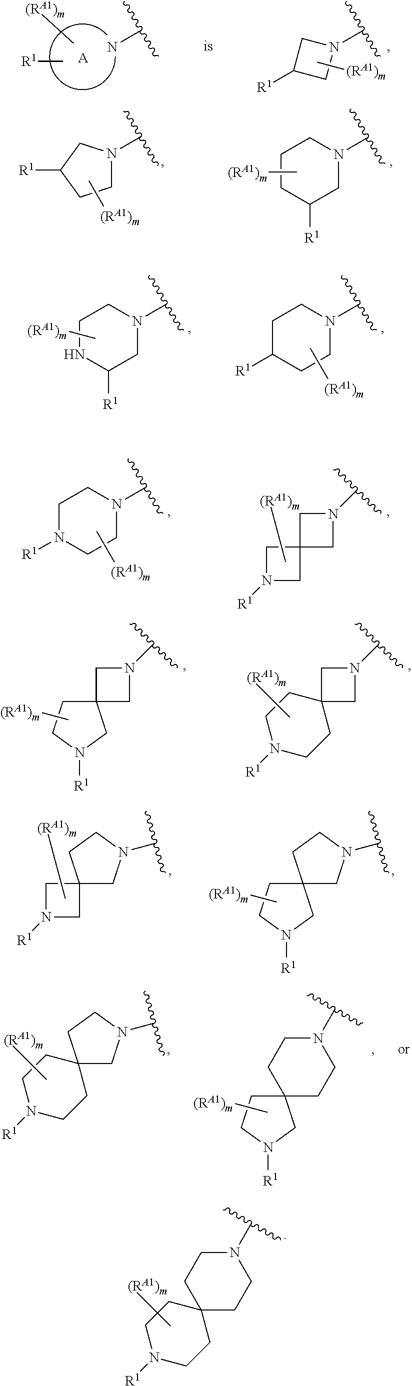

In some embodiments,

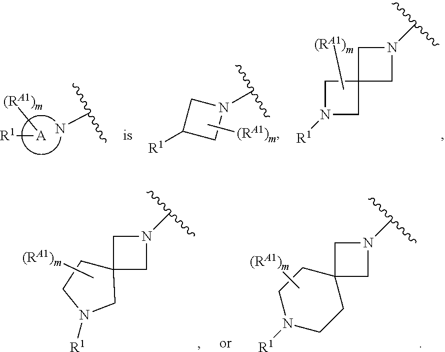

In some embodiments, m is 0, 1 or 2. In some embodiments, m is 0. In some embodiments, m is 1. In some embodiments, m is 2.

In some embodiments,

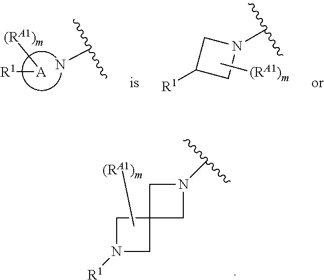

In some embodiments, m is 0, 1 or 2. In some embodiments, m is 0. In some embodiments, m is 1. In some embodiments, m is 2.

In some embodiments,

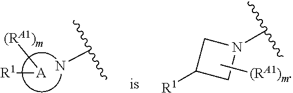

In some embodiments, m is 0, 1 or 2. In some embodiments, m is 0. In some embodiments, m is 1. In some embodiments, m is 2.

In some embodiments,

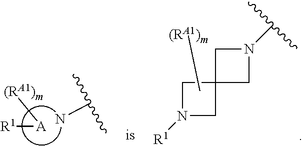

In some embodiments, m is 0, 1 or 2. In some embodiments, m is 0. In some embodiments, m is 1. In some embodiments, m is 2.

In some embodiments of a compound of Formula (A) or Formula (I), or a pharmaceutically acceptable salt or solvate thereof, each R^{A1} is independently F, —CN, —OR³, or substituted or unsubstituted C₁-C₄alkyl; and R³ is hydrogen, substituted or unsubstituted C₁-C₄alkyl, or substituted or unsubstituted C₃-C₆cycloalkyl. In some embodiments, each R^{A1} is independently F, —OH, —CH₃, or —OCH₃. In some embodiments, each R^{A1} is independently F, —CH₃, or —OCH₃. In some embodiments, each R^{A1} is independently —CH₃ or —OCH₃. In some embodiments, R^{A1} is F. In some embodiments, R^{A1} is —OH. In some embodiments, R^{A1} is —CH₃. In some embodiments, R^{A1} is —OCH₃.

In some embodiments of a compound of Formula (A) or Formula (I), or a pharmaceutically acceptable salt or solvate thereof, m is 1 or 2. In some embodiments, m is 0. In some embodiments, m is 1. In some embodiments, m is 2.

In some embodiments of a compound of Formula (A) or Formula (I), or a pharmaceutically acceptable salt or solvate thereof, R¹ is —OR³, —SR³, —C(═O)R³, —C(═O)NR³R⁴, —C(═O)OR³, —NR³R⁴, substituted or unsubstituted C₁-C₆ alkyl, or substituted or unsubstituted C₁-C₆ aminoalkyl; and each R³ and R⁴ is independently hydrogen, substituted or unsubstituted C₁-C₄alkyl, or substituted or unsubstituted C₃-C₆cycloalkyl. In some embodiments, R¹ is substituted or unsubstituted C₁-C₆ alkyl. In some embodiments, R¹ is C₁-C₄ alkyl substituted with —OR³ or —NR³R⁴. In some embodiments, each R³ and R⁴ is independently hydrogen or C₁-C₄ alkyl. In some embodiments, R¹ is C₁-C₄ alkyl substituted with —OH or —NH₂. In some embodiments, R¹ is —CH₂OH, —CH₂CH₂OH, —CH₂NH₂, or —CH₂CH₂NH₂. In some embodiments, R¹ is —OR³ or —NR³R⁴. In some embodiments, each R³ and R⁴ is independently hydrogen or C₁-C₄ alkyl. In some embodiments, each R³ and R⁴ is hydrogen. In some embodiments, each R³ and R⁴ is independently C₁-C₄ alkyl. In some embodiments, R³ is hydrogen and R⁴ is C₁-C₄ alkyl. In some embodiments, the C₁-C₄ alkyl is selected from —CH₃, —CH₂CH₃, —CH₂CH₂CH₃, and —CH(CH₃)₂. In some embodiments, R¹ is —NH₂ or —OH. In some embodiments, R¹ is —NH₂. In some embodiments, R¹ is —OH.

In another aspect, the present disclosure provides a compound of Formula (II), or a pharmaceutically acceptable salt or solvate thereof:

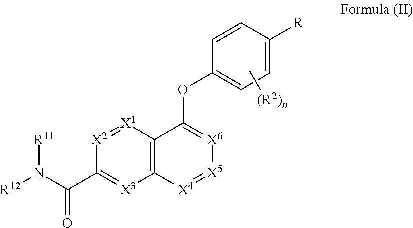

wherein,
X¹ is N or CR^{X1}; X² is N or CR^{X2}; X³ is N or CR^{X3}; X⁴ is N or CR^{X4}; X⁵ is N or CR^{X5}; X⁶ is N or CR^{X6};
each R^{X1}, R^{X2}, R^{X3}, R^{X4}, R^{X5}, and R^{X6} is independently hydrogen, halogen, nitro, —OR³, —SR³, —CN, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₇cycloalkyl, or substituted or unsubstituted C₂-C₆heterocycloalkyl;
R is halogen, nitro, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, or substituted or unsubstituted C₁-C₆fluoroalkyl;
each R¹¹ and R¹² is independently —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R² is independently halogen, nitro, —CN, —OR³, —SR³, —S(═O)₂R³, —NR³R⁴, —C(═O)OR³, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; each R³ and R⁴ is independently hydrogen, —CN, substituted or unsubstituted C₁-C₆alkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or R³ and R⁴ on the same nitrogen atom taken together with the nitrogen atom to which they are attached to form a substituted or unsubstituted N-containing C₃-C₇ heterocycloalkyl; and
n is 0, 1, 2, 3, or 4.

In some embodiments of a compound of Formula (A) or Formula (II), or a pharmaceutically acceptable salt or solvate thereof, each R¹¹ and R¹² is independently —CN, —OR³, —SR³, —C(═O)R³, —C(═O)NR³R⁴, —C(═O)OR³, —S(═O)R³, —S(═O)₂R³, —NR³R⁴, substituted or unsubstituted C₁-C₆alkyl, substituted or unsubstituted C₁-C₆aminoalkyl, or substituted or unsubstituted C₃-C₁₀cycloalkyl; and each R³ and R⁴ is independently hydrogen, substituted or unsubstituted C₁-C₄alkyl, or substituted or unsubstituted C₃-C₆cycloalkyl. In some embodiments, each R¹¹ and R¹² is independently —CN, —OR³, —S(═O)₂R³, —NR³R⁴, substituted or unsubstituted C₁-C₆alkyl, or substituted or unsubstituted C₁-C₆aminoalkyl. In some embodiments, each R¹¹ and R¹² is independently —CN or substituted or unsubstituted C₁-C₆alkyl. In some embodiments, each R¹¹ and R¹² is independently —CN, —CH₃, —CH₂CH₃, or —CH₂CH₂CH₃.

In some embodiments of a compound of Formula (A), Formula (I), or Formula (II), or a pharmaceutically acceptable salt or solvate thereof, X¹ is CR^{X1}; X¹ is CR^{X2}; and X³ is CR^{X3}. In some embodiments, X¹ is N; X¹ is CR²; and X³ is CR^{X3}. In some embodiments, X¹ is CR^{X1}; X¹ is CR^{X2}; and X³ is N.

In some embodiments of a compound of Formula (A), Formula (I), or Formula (II), or a pharmaceutically acceptable salt or solvate thereof, X⁴ is CR^{X4}; X⁵ is CR^{X5}; and X⁶ is CR^{X6}. In some embodiments, X⁴ is CR^{X4}; X⁵ is CR^{X5}; and X⁶ is N.

In some embodiments of a compound has the following structure, or a pharmaceutically acceptable salt or solvate thereof:

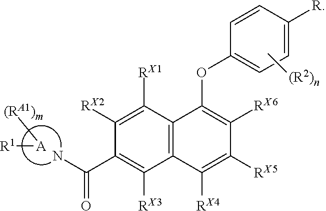

In some embodiments of a compound has the following structure, or a pharmaceutically acceptable salt or solvate thereof:

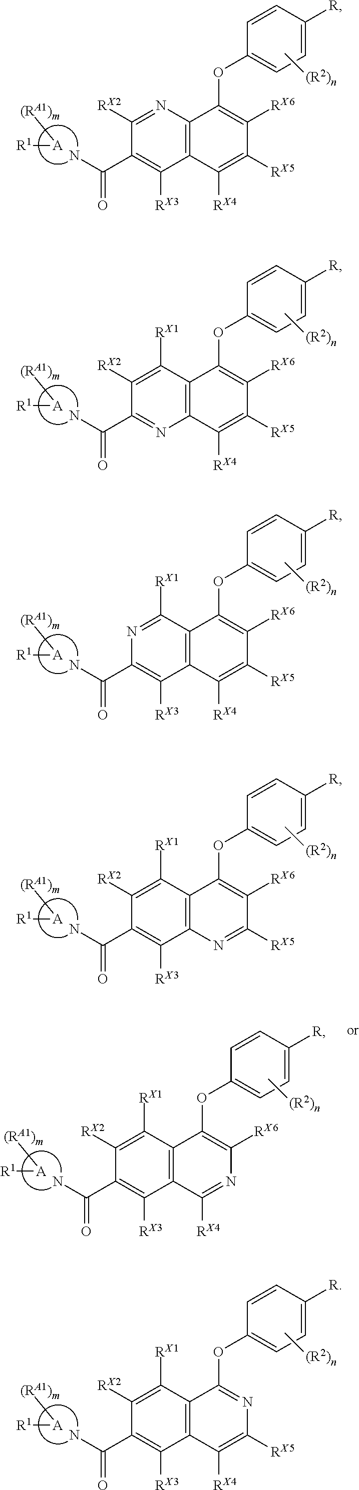

In some embodiments of a compound has the following structure, or a pharmaceutically acceptable salt or solvate thereof:

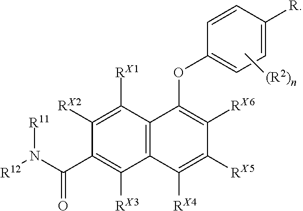

In some embodiments of a compound has the following structure, or a pharmaceutically acceptable salt or solvate thereof:

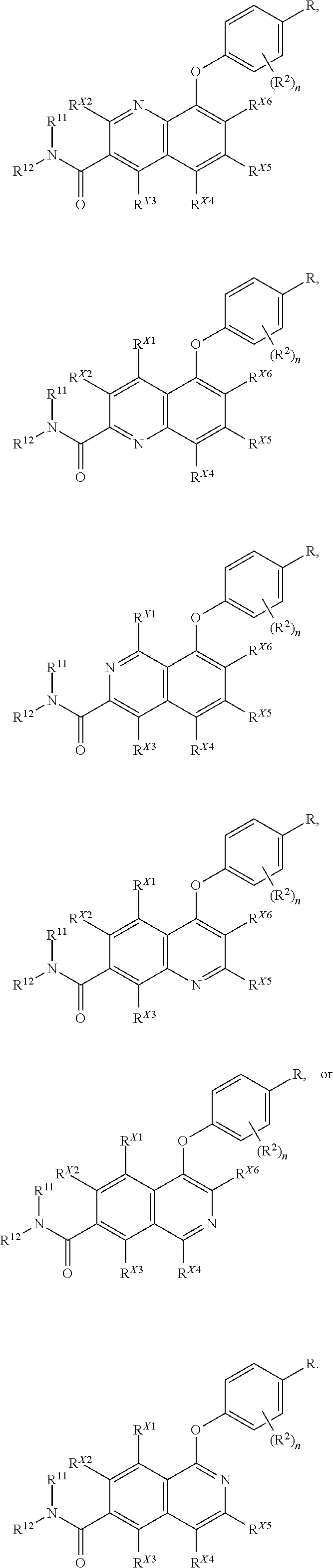

In some embodiments of a compound of Formula (A), Formula (I), or Formula (II), or a pharmaceutically acceptable salt or solvate thereof, each R^{X1}, R^{X2}, R^{X3}, R^{X4}, R^{X5}, and R^{X6}, when present, is independently hydrogen, halogen, —OR³, —SR³, —CN, —S(═O)R³, —S(═O)₂R³, —NR³R⁴, —NR³S(═O)₂R³, —NR³C(═O)R³, substituted or unsubstituted C₁-C₆alkyl, substituted or unsubstituted C₁-C₆fluoroalkyl, substituted or unsubstituted C₂-C₄alkenyl, substituted or unsubstituted C₂-C₄alkynyl, or substituted or unsubstituted C₁-C₆heteroalkyl; and each R³ and R⁴ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, or substituted or unsubstituted C₂-C₁₀heterocycloalkyl; or R³ and R⁴ are taken together with the nitrogen atom to which they are attached to form a substituted or unsubstituted C₃-C₇ heterocycloalkyl. In some embodiments, each R^{X1}, R^{X2}, R^{X3}, R^{X4}, R^{X5}, and R^{X6}, when present, is independently hydrogen, F, Cl, Br, I, —CH₃, —CH₂CH₃, cyclopropyl, —C≡CH, —OH, —OCH₃, —OCH₂CH₃, —OCF₃, —SCH₃, cyclopropyloxy, —NH₂, —NHC(═O)CH₃, —N(CH₃)C(═O)CH₃, —NHS(═O)₂CH₃, —N(CH₃)S(═O)₂CH₃, —S(═O)CH₃, or —S(═O)₂CH₃. In some embodiments, each R^{X1}, R^{X2}, R^{X3}, R^{X4}, R^{X5}, and R^{X6}, when present, is independently hydrogen, F, Cl, Br, —CH₃, —OH, —OCH₃, or —OCF₃. In some embodiments, each R^{X1}, R^{X2}, R^{X3}, R^{X4}, R^{X5}, and R^{X6}, when present, is independently hydrogen, F, or —OCH₃. In some embodiments, each R^{X1}, R^{X2}, R^{X3}, R^{X4}, R^{X5}, and R^{X6}, when present, is hydrogen. In some embodiments, one of R^{X1}, R^{X2}, R^{X3}, R^{X4}, R^{X5}, and R^{X6}, is hydrogen. In some embodiments, two of R^{X1}, R^{X2}, R^{X3}, R^{X4}, R^{X5}, and R^{X6}, is hydrogen. In some embodiments, three of R^{X1}, R^{X2}, R^{X3}, R^{X4}, R^{X5} and R^{X6}, is hydrogen. In some embodiments, four of R^{X1}, R^{X2}, R^{X3}, R^{X4}, R^{X5}, and R^{X6}, is hydrogen. In some embodiments, five of Rx, R^{X2}, R^{X3}, R^{X4}, R^{X5}, and R^{X6}, is hydrogen.

In some embodiments, R^{X1}, when present, is hydrogen. In some embodiments, R^{X2} when present, is hydrogen. In some embodiments, R^{X3}, when present, is hydrogen. In some embodiments, R^{X4}, when present, is hydrogen. In some embodiments, R^{X5}, when present, is hydrogen. In some embodiments, R^{X6}, when present, is hydrogen.

In some embodiments of a compound of Formula (A), Formula (I), or Formula (II), or a pharmaceutically acceptable salt or solvate thereof, R is halogen, nitro, —CN, —OR³, —C(═O)R³, —C(═O)NR³R⁴, —C(═O)OR³, —S(═O)R³, —S(═O)₂R³, —NR³S(═O)₂R³, —NR³C(═O)R³, —NR³C(═O)OR³, or substituted or unsubstituted C₁-C₆fluoroalkyl; and each R³ and R⁴ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, or substituted or unsubstituted C₂-C₁₀heterocycloalkyl; or R³ and R⁴ are taken together with the nitrogen atom to which they are attached to form a substituted or unsubstituted C₃-C₇ heterocycloalkyl. In some embodiments, R is F, Cl, Br, I, nitro, —CN, —OCH₂F, —OCHF₂, —OCF₃, —C(═O)CH₃, —C(═O)OCH₃—C(═O)NH₂, —C(═O)NHCH₃, —C(═O)N(CH₃)₂, —S(═O)CH₃, —S(═O)₂CH₃, —NHS(═O)₂CH₃, —N(CH₃)S(═O)₂CH₃, —NHC(═O)CH₃, —N(CH₃)C(═O)CH₃, —NHC(═O)OCH₃, —N(CH₃)C(═O)OCH₃, —CH₂F, —CHF₂, or —CF₃. In some embodiments, R is F, Cl, —CN, —OCF₃, —CHF₂, or —CF₃. In some embodiments, R is F, Cl, —OCF₃, —CHF₂, or —CF₃. In some embodiments, R is —OCF₃, —CHF₂, or —CF₃. In some embodiments, R is —CF₃.

In some embodiments of a compound of Formula (A), Formula (I), or Formula (II), or a pharmaceutically acceptable salt or solvate thereof, each R² is independently halogen, nitro, —CN, —OR³, or substituted or unsubstituted C₁-C₆alkyl; and each R³ is independently hydrogen, substituted or unsubstituted C₁-C₆alkyl, or substituted or unsubstituted C₁-C₆fluoroalkyl. In some embodiments, each R² is independently F, Cl, —CN, —OCH₃, —OCF₃, or —CF₃. In some embodiments, each R² is independently F, Cl, —OCF₃, or —CF₃. In some embodiments, each R² is independently F or Cl.

In another aspect, the present disclosure provides a compound or pharmaceutically acceptable salt thereof, wherein the compound is a compound from Table 1, or a pharmaceutically acceptable salt or solvate thereof.

TABLE 1

| Compound | | |
|---|---|---|
| # | Structure | Name |
| 1A | 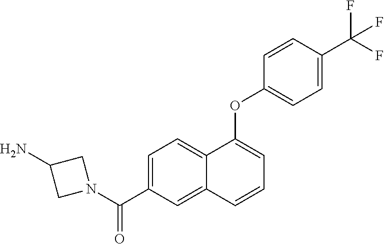 [SMILES: NC1CN(C(=O)c2ccc3c(Oc4ccc(C(F)(F)F)cc4)cccc3c2)C1] | (3-aminoazetidin-1-yl)(5-(4- (trifluoromethyl)phenoxy)naphthalen- 2-yl)methanone |
| 1 | 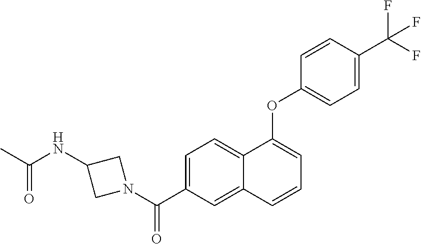 [SMILES: CC(=O)NC1CN(C(=O)c2ccc3c(Oc4ccc(C(F)(F)F)cc4)cccc3c2)C1] | N-(1-(5-(4- (Trifluoromethyl)phenoxy)-2- naphthoyl)azetidin-3-yl)acetamide |
| 2 | 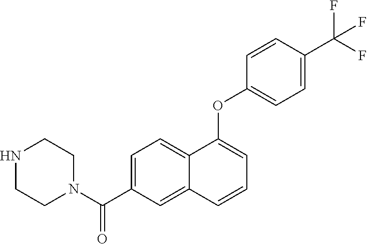 [SMILES: O=C(c1ccc2c(Oc3ccc(C(F)(F)F)cc3)cccc2c1)N1CCNCC1] | Piperazin-1-yl(5-(4- (trifluoromethyl)phenoxy)naphthalen- 2-yl)methanone |
| 3 | 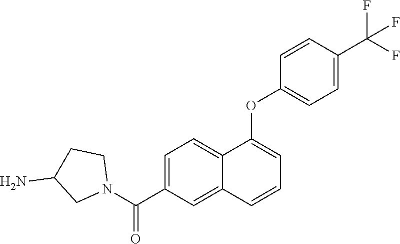 [SMILES: NC1CCN(C(=O)c2ccc3c(Oc4ccc(C(F)(F)F)cc4)cccc3c2)C1] | (3-Aminopyrrolidin-1-yl)(5-(4- (trifluoromethyl)phenoxy)naphthalen- 2-yl)methanone |
| 4 | 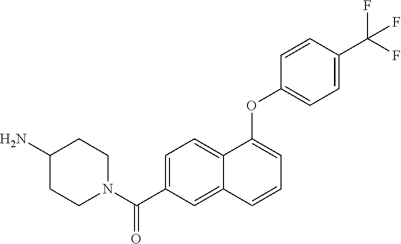 [SMILES: NC1CCN(C(=O)c2ccc3c(Oc4ccc(C(F)(F)F)cc4)cccc3c2)CC1] | (4-Aminopiperidin-1-yl)(5-(4- (trifluoromethyl)phenoxy)naphthalen- 2-yl)methanone |
| 5 | 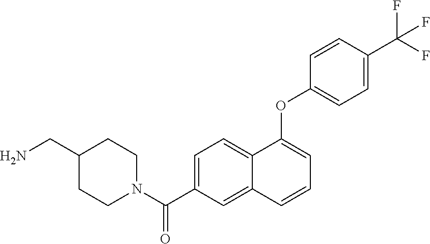 [SMILES: NCC1CCN(C(=O)c2ccc3c(Oc4ccc(C(F)(F)F)cc4)cccc3c2)CC1] | (4-(Aminomethyl)piperidin-1-yl)(5-(4- (trifluoromethyl)phenoxy)naphthalen- 2-yl)methanone |
| 6 | 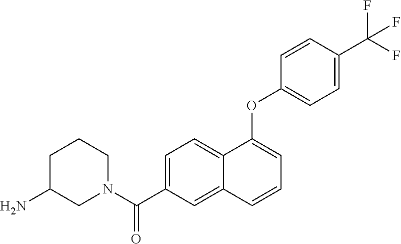 [SMILES: NC1CCCN(C(=O)c2ccc3c(Oc4ccc(C(F)(F)F)cc4)cccc3c2)C1] | (3-Aminopiperidin-1-yl)(5-(4- (trifluoromethyl)phenoxy)naphthalen- 2-yl)methanone |
| 7 | 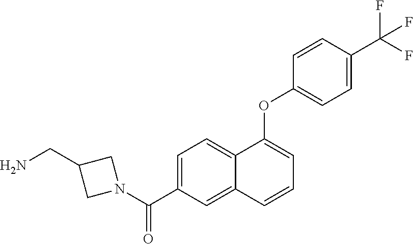 [SMILES: NCC1CN(C(=O)c2ccc3c(Oc4ccc(C(F)(F)F)cc4)cccc3c2)C1] | (3-(Aminomethyl)azetidin-1-yl)(5-(4- (trifluoromethyl)phenoxy)naphthalen- 2-yl)methanone |
| 8 | 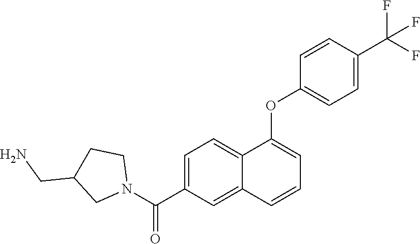 [SMILES: NCC1CCN(C(=O)c2ccc3c(Oc4ccc(C(F)(F)F)cc4)cccc3c2)C1] | (3-(Aminomethyl)pyrrolidin-1-yl)(5- (4- (trifluoromethyl)phenoxy)naphthalen- 2-yl)methanone |
| 9 | 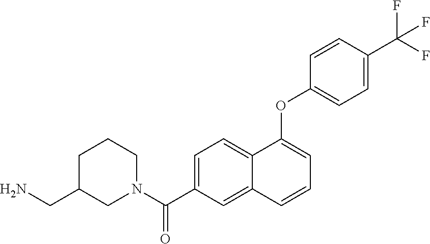 [SMILES: NCC1CCCN(C(=O)c2ccc3c(Oc4ccc(C(F)(F)F)cc4)cccc3c2)C1] | (3-(Aminomethyl)piperidin-1-yl)(5-(4- (trifluoromethyl)phenoxy)naphthalen- 2-yl)methanone |
| 10 | 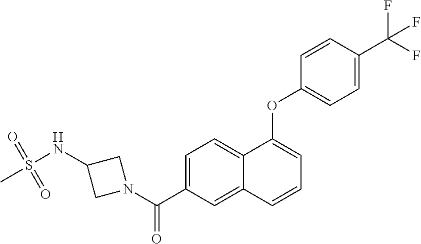 [SMILES: CS(=O)(=O)NC1CN(C(=O)c2ccc3c(Oc4ccc(C(F)(F)F)cc4)cccc3c2)C1] | N-((1-(5-(4- (trifluoromethyl)phenoxy)-2- naphthoyl)azetidin-3- yl)methyl)acetamide |
| 11 | 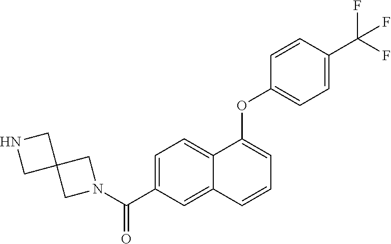 [SMILES: O=C(c1ccc2c(Oc3ccc(C(F)(F)F)cc3)cccc2c1)N1CC2(CNC2)C1] | (2,6-Diazaspiro[3.3]heptan-2-yl)(5-(4- (trifluoromethyl)phenoxy)naphthalen- 2-yl)methanone |
| 12 | 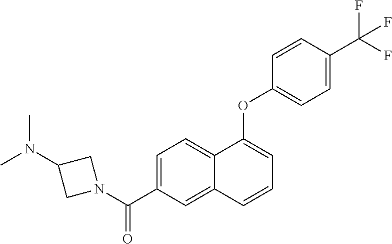 [SMILES: CN(C)C1CN(C(=O)c2ccc3c(Oc4ccc(C(F)(F)F)cc4)cccc3c2)C1] | (3-(Dimethylamino)azetidin-1-yl)(5- (4- (trifluoromethyl)phenoxy)naphthalen- 2-yl)methanone |
| 13 | 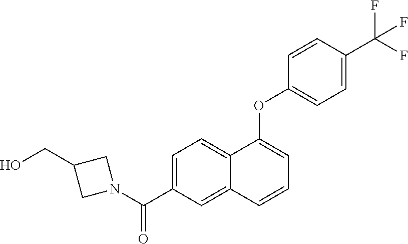 [SMILES: O=C(c1ccc2c(Oc3ccc(C(F)(F)F)cc3)cccc2c1)N1CC(CO)C1] | (3-(Hydroxymethyl)azetidin-1-yl)(5- (4- (trifluoromethyl)phenoxy)naphthalen- 2-yl)methanone |
| 14 | 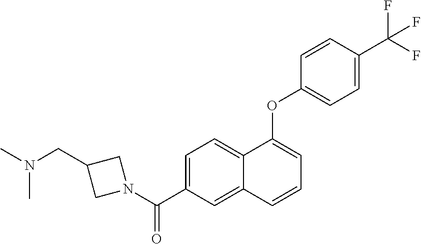 [SMILES: CN(C)CC1CN(C(=O)c2ccc3c(Oc4ccc(C(F)(F)F)cc4)cccc3c2)C1] | (3-((Dimethylamino)methyl)azetidin- 1-yl)(5-(4- (trifluoromethyl)phenoxy)naphthalen- 2-yl)methanone |
| 15 | 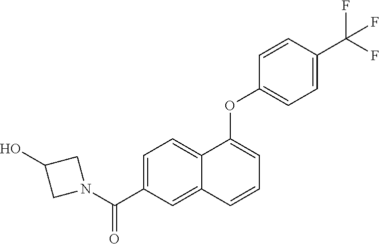 [SMILES: O=C(c1ccc2c(Oc3ccc(C(F)(F)F)cc3)cccc2c1)N1CC(O)C1] | (3-Hydroxyazetidin-1-yl)(5-(4- (trifluoromethyl)phenoxy)naphthalen- 2-yl)methanone |
| 16 | 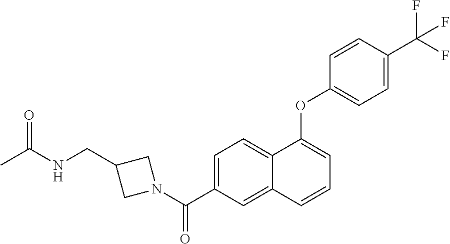 [SMILES: CC(=O)NCC1CN(C(=O)c2ccc3c(Oc4ccc(C(F)(F)F)cc4)cccc3c2)C1] | N-((1-(5-(4- (trifluoromethyl)phenoxy)-2- naphthoyl)azetidin-3- yl)methyl)acetamide |
| 17 | 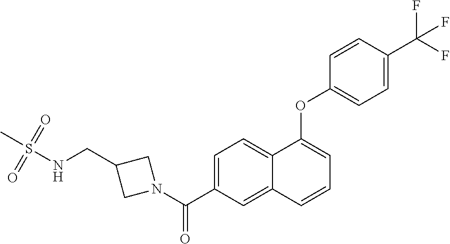 [SMILES: CS(=O)(=O)NCC1CN(C(=O)c2ccc3c(Oc4ccc(C(F)(F)F)cc4)cccc3c2)C1] | N-((1-(5-(4- (trifluoromethyl)phenoxy)-2- naphthoyl)azetidin-3- yl)methyl)methanesulfonamide |
| 18 | 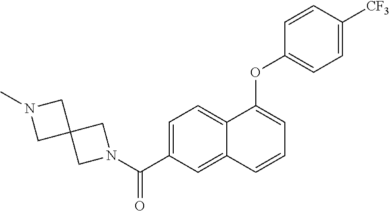 [SMILES: CN1CC2(C1)CN(C(=O)c1ccc3c(Oc4ccc(C(F)(F)F)cc4)cccc3c1)C2] | (6-Methyl-2,6-diazaspiro[3.3]heptan- 2-yl)(5-(4- (trifluoromethyl)phenoxy)naphthalen- 2-yl)methanone |
| 19 | 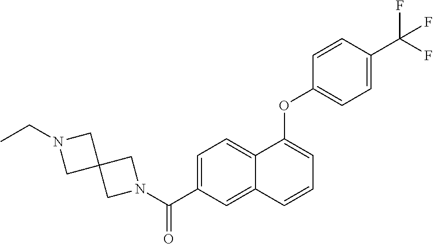 [SMILES: CCN1CC2(C1)CN(C(=O)c1ccc3c(Oc4ccc(C(F)(F)F)cc4)cccc3c1)C2] | (6-Ethyl-2,6-diazaspiro[3.3]heptan-2- yl)(5-(4- (trifluoromethyl)phenoxy)naphthalen- 2-yl)methanone |
| 20 | 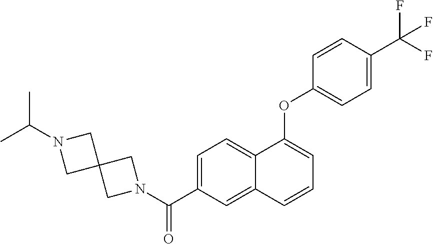 [SMILES: CC(C)N1CC2(CN(C(=O)c3ccc4c(Oc5ccc(C(F)(F)F)cc5)cccc4c3)C2)C1] | (6-Isopropyl-2,6- diazaspiro[3.3]heptan-2-yl)(5-(4- (trifluoromethyl)phenoxy)naphthalen- 2-yl)methanone |
| 21 | 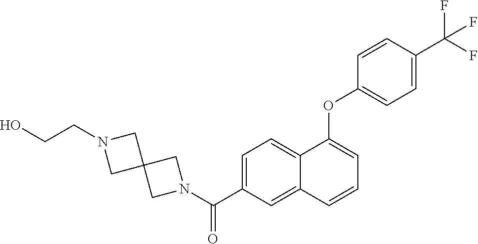 [SMILES: O=C(c1ccc2c(Oc3ccc(C(F)(F)F)cc3)cccc2c1)N1CC2(CN(CCO)C2)C1] | (6-(2-Hydroxyethyl)-2,6- diazaspiro[3.3]heptan-2-yl)(5-(4- (trifluoromethyl)phenoxy)naphthalen- 2-yl)methanone |
| 22 | 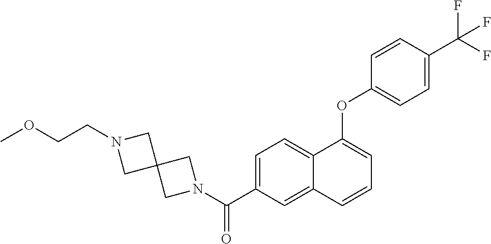 [SMILES: COCCN1CC2(C1)CN(C(=O)c1ccc3c(Oc4ccc(C(F)(F)F)cc4)cccc3c1)C2] | (6-(2-Methoxyethyl)-2,6- diazaspiro[3.3]heptan-2-yl)(5-(4- (trifluoromethyl)phenoxy)naphthalen- 2-yl)methanone |
| 23 | 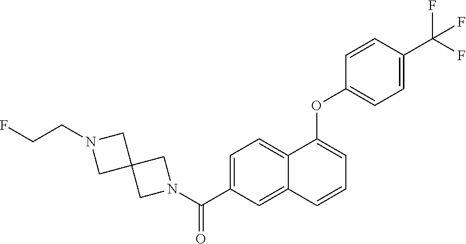 [SMILES: O=C(c1ccc2c(Oc3ccc(C(F)(F)F)cc3)cccc2c1)N1CC2(CN(CCF)C2)C1] | (6-(2-Fluoroethyl)-2,6- diazaspiro[3.3]heptan-2-yl)(5-(4- (trifluoromethyl)phenoxy)naphthalen- 2-yl)methanone |
| 24 | 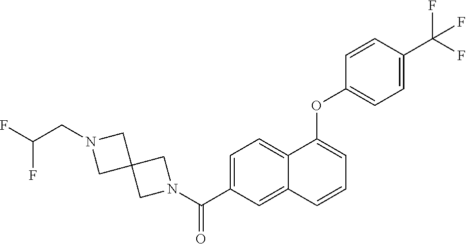 [SMILES: O=C(c1ccc2c(Oc3ccc(C(F)(F)F)cc3)cccc2c1)N1CC2(CN(CC(F)F)C2)C1] | (6-(2,2-Difluoroethyl)-2,6- diazaspiro[3.3]heptan-2-yl)(5-(4- (trifluoromethyl)phenoxy)naphthalen- 2-yl)methanone |
| 25 | 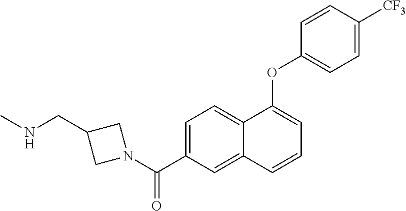 [SMILES: CNCC1CN(C(=O)c2ccc3c(Oc4ccc(C(F)(F)F)cc4)cccc3c2)C1] | (3-((Methylamino)methyl)azetidin-1- yl)(5-(4- (trifluoromethyl)phenoxy)naphthalen- 2-yl)methanone |
| 26 | 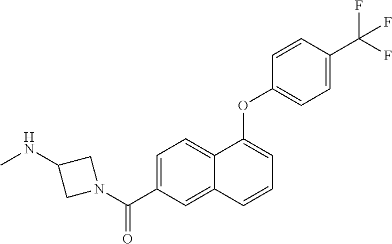 [SMILES: CNC1CN(C(=O)c2ccc3c(Oc4ccc(C(F)(F)F)cc4)cccc3c2)C1] | (3-(Methylamino)azetidin-1-yl)(5-(4- (trifluoromethyl)phenoxy)naphthalen- 2-yl)methanone |
| 27 | 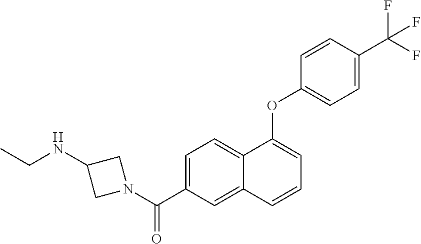 [SMILES: CCNC1CN(C(=O)c2ccc3c(Oc4ccc(C(F)(F)F)cc4)cccc3c2)C1] | (3-(Ethylamino)azetidin-1-yl)(5-(4- (trifluoromethyl)phenoxy)naphthalen- 2-yl)methanone |
| 28 | 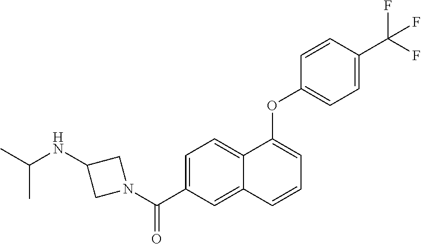 [SMILES: CC(C)NC1CN(C(=O)c2ccc3c(Oc4ccc(C(F)(F)F)cc4)cccc3c2)C1] | (3-(Isopropylamino)azetidin-1-yl)(5- (4- (trifluoromethyl)phenoxy)naphthalen- 2-yl)methanone |
| 29 | 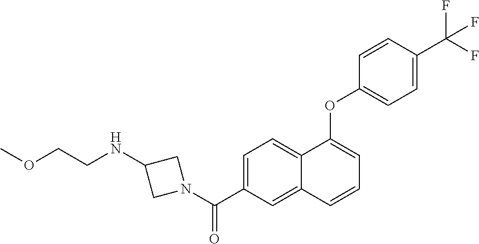 [SMILES: COCCNC1CN(C(=O)c2ccc3c(Oc4ccc(C(F)(F)F)cc4)cccc3c2)C1] | (3-((2-Methoxyethyl)amino)azetidin- 1-yl)(5-(4- (trifluoromethyl)phenoxy)naphthalen- 2-yl)methanone |
| 30 | 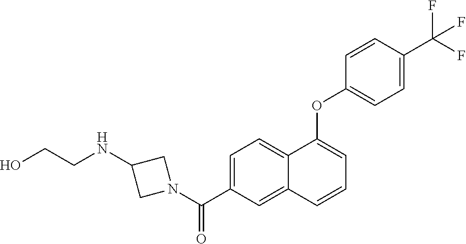 [SMILES: O=C(c1ccc2c(Oc3ccc(C(F)(F)F)cc3)cccc2c1)N1CC(NCCO)C1] | (3-((2-Hydroxyethyl)amino)azetidin-1- yl)(5-(4- (trifluoromethyl)phenoxy)naphthalen- 2-yl)methanone |
| 31 | 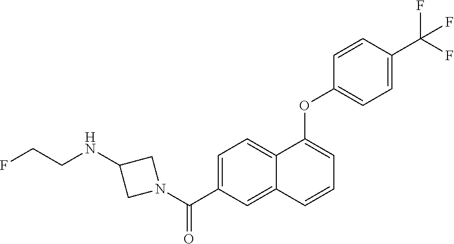 [SMILES: O=C(c1ccc2c(Oc3ccc(C(F)(F)F)cc3)cccc2c1)N1CC(NCCF)C1] | (3-((2-Fluoroethyl)amino)azetidin-1- yl)(5-(4- (trifluoromethyl)phenoxy)naphthalen- 2-yl)methanone |
| 32 | 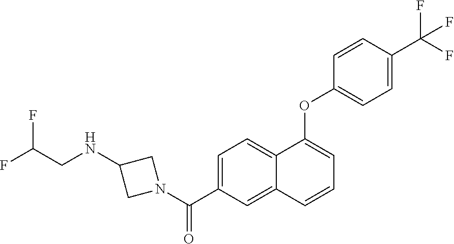 [SMILES: O=C(c1ccc2c(Oc3ccc(C(F)(F)F)cc3)cccc2c1)N1CC(NCC(F)F)C1] | (3-((2,2-Difluoroethyl)amino)azetidin- 1-yl)(5-(4- (trifluoromethyl)phenoxy)naphthalen- 2-yl)methanone |
| 33 | 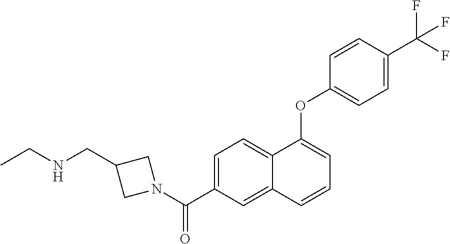 [SMILES: CCNCC1CN(C(=O)c2ccc3c(Oc4ccc(C(F)(F)F)cc4)cccc3c2)C1] | (3-((Ethylamino)methyl)azetidin-1- yl)(5-(4- (trifluoromethyl)phenoxy)naphthalen- 2-yl)methanone |
| 34 | 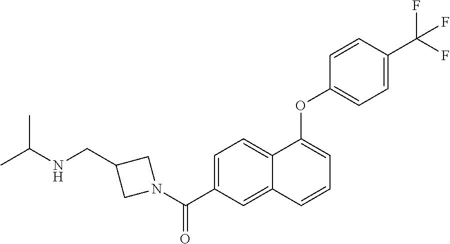 [SMILES: CC(C)NCC1CN(C(=O)c2ccc3c(Oc4ccc(C(F)(F)F)cc4)cccc3c2)C1] | (3-((Isopropylamino)methyl)azetidin- 1-yl)(5-(4- (trifluoromethyl)phenoxy)naphthalen- 2-yl)methanone |
| 35 | 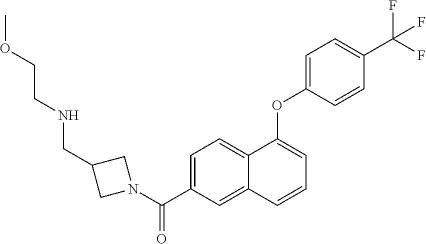 [SMILES: COCCNCC1CN(C(=O)c2ccc3c(Oc4ccc(C(F)(F)F)cc4)cccc3c2)C1] | (3-(((2- Methoxyethyl)amino)methyl)azetidin- 1-yl)(5-(4- (trifluoromethyl)phenoxy)naphthalen- 2-yl)methanone |
| 36 | 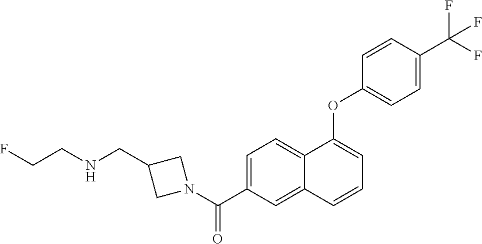 [SMILES: O=C(c1ccc2c(Oc3ccc(C(F)(F)F)cc3)cccc2c1)N1CC(CNCCF)C1] | (3-(((2- Fluoroethyl)amino)methyl)azetidin-1- yl)(5-(4- (trifluoromethyl)phenoxy)naphthalen- 2-yl)methanone |
| 37 | 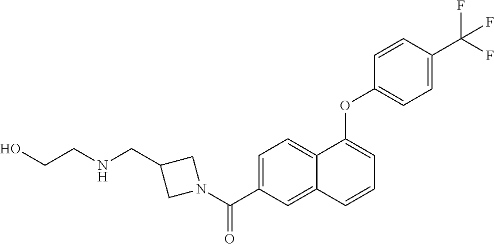 [SMILES: O=C(c1ccc2c(Oc3ccc(C(F)(F)F)cc3)cccc2c1)N1CC(CNCCO)C1] | (3-(((2- Hydroxyethyl)amino)methyl)azetidin- 1-yl)(5-(4- (trifluoromethyl)phenoxy)naphthalen- 2-yl)methanone |
| 38 | 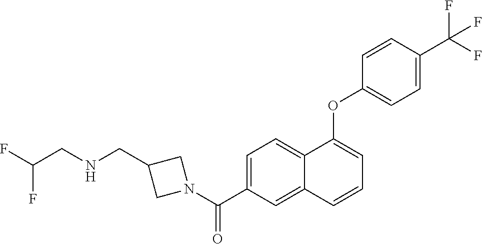 [SMILES: O=C(c1ccc2c(Oc3ccc(C(F)(F)F)cc3)cccc2c1)N1CC(CNCC(F)F)C1] | (3-(((2,2- Fifluoroethyl)amino)methyl)azetidin- 1-yl)(5-(4- (trifluoromethyl)phenoxy)naphthalen- 2-yl)methanone |
| 39 | 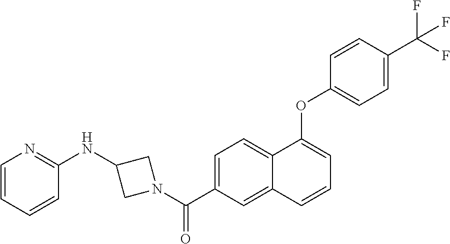 [SMILES: O=C(c1ccc2c(Oc3ccc(C(F)(F)F)cc3)cccc2c1)N1CC(Nc2ccccn2)C1] | (3-(Pyridin-2-ylamino)azetidin-1- yl)(5-(4- (trifluoromethyl)phenoxy)naphthalen- 2-yl)methanone |
| 40 | 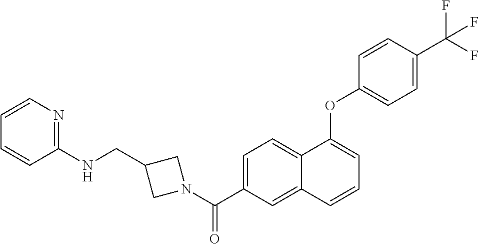 [SMILES: O=C(c1ccc2c(Oc3ccc(C(F)(F)F)cc3)cccc2c1)N1CC(CNc2ccccn2)C1] | (3-((Pyridin-2- ylamino)methyl)azetidin-1-yl)(5-(4- (trifluoromethyl)phenoxy)naphthalen- 2-yl)methanone |
| 41 | 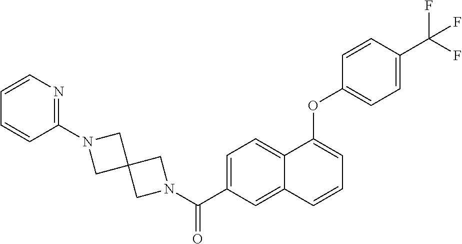 [SMILES: O=C(c1ccc2c(Oc3ccc(C(F)(F)F)cc3)cccc2c1)N1CC2(C1)CN(c1ccccn1)C2] | (6-(Pyridin-2-yl)-2,6- diazaspiro[3.3]heptan-2-yl)(5-(4- (trifluoromethyl)phenoxy)naphthalen- 2-yl)methanone |
| 42 | 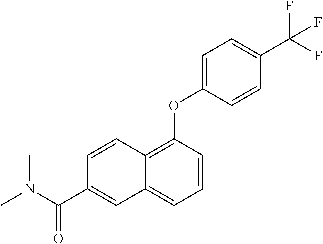 [SMILES: CN(C)C(=O)c1ccc2c(Oc3ccc(C(F)(F)F)cc3)cccc2c1] | N,N-dimethyl-5-(4- (trifluoromethyl)phenoxy)naphthalene- 2-carboxamide |
| 43 | 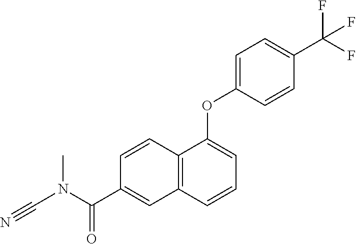 [SMILES: CN(C#N)C(=O)c1ccc2c(Oc3ccc(C(F)(F)F)cc3)cccc2c1] | N-cyano-N-methyl-5-(4- (trifluoromethyl)phenoxy)-2- naphthamide |

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Methods known to one of ordinary skill in the art are identified through various reference books and databases. Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

In some instances, specific and analogous reactants are identified through the indices of known chemicals prepared by the Chemical Abstract Service of the American Chemical Society, which are available in most public and university libraries, as well as through on-line databases (the American Chemical Society, Washington, D.C., is contacted for more details). Chemicals that are known but not commercially available in catalogs are prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In some embodiments, the compounds disclosed herein are prepared as described in the Examples section.

### Further Forms of Compounds

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, disclosed herein are dissociable complexes (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. In some embodiments, examples of isotopes that are incorporated into compounds of the disclosure include isotopes of hydrogen, carbon, nitrogen, oxygen, phosphorous, sulfur, fluorine, and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvates, hydrates, or derivatives thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the disclosure, or by separately reacting a purified compound in its free form with a suitable acid or base, and isolating the salt thus formed.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. In some embodiments, solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or methanol. In some embodiments, the compounds provided herein exist in unsolvated as well as solvated forms. In general, the solvated forms are considered equivalent to the unsolvated forms for the purposes of the compounds and methods provided herein.

### Prodrugs

In some embodiments, the compounds described herein exist in prodrug form. The disclosure provides for methods of treating diseases by administering such prodrugs. The disclosure further provides for methods of treating diseases by administering such prodrugs as pharmaceutical compositions.

In some embodiments, prodrugs include compounds wherein an amino acid residue, or a polypeptide chain of two or more (e. g., two, three, or four) amino acid residues is covalently joined through an amide or ester bond to a free amino, hydroxy, or carboxylic acid group of compounds of the present disclosure. The amino acid residues include, but are not limited to, the 20 naturally occurring amino acids and also includes 4-hydroxyproline, hydroxylysine, demosine, isodemosine, 3-methylhistidine, norvaline, beta-alanine, gamma-aminobutyric acid, citrulline, homocysteine, homoserine, ornithine, and methionine sulfone. In other embodiments, prodrugs include compounds wherein a nucleic acid residue, or an oligonucleotide of two or more (e. g., two, three or four) nucleic acid residues is covalently joined to a compound of the present disclosure.

Pharmaceutically acceptable prodrugs of the compounds described herein also include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, metal salts, and sulfonate esters. In some embodiments, compounds having free amino, amido, hydroxy, or carboxylic groups are converted into prodrugs. For instance, free carboxyl groups are derivatized as amides or alkyl esters. In certain instances, all of these prodrug moieties incorporate groups including, but not limited to, ether, amine, and carboxylic acid functionalities.

Hydroxy prodrugs include esters such as, though not limited to, acyloxyalkyl (e.g. acyloxymethyl, acyloxyethyl) esters, alkoxycarbonyloxyalkyl esters, alkyl esters, aryl esters, sulfonate esters, sulfate esters and disulfide containing esters, ethers, amides, carbamates, hemisuccinates, dimethylaminoacetates, and phosphoryloxymethyloxycarbonyls, as outlined in Advanced Drug Delivery Reviews 1996, 19, 115.

Amine derived prodrugs include, but are not limited to, the following groups and combinations of groups:

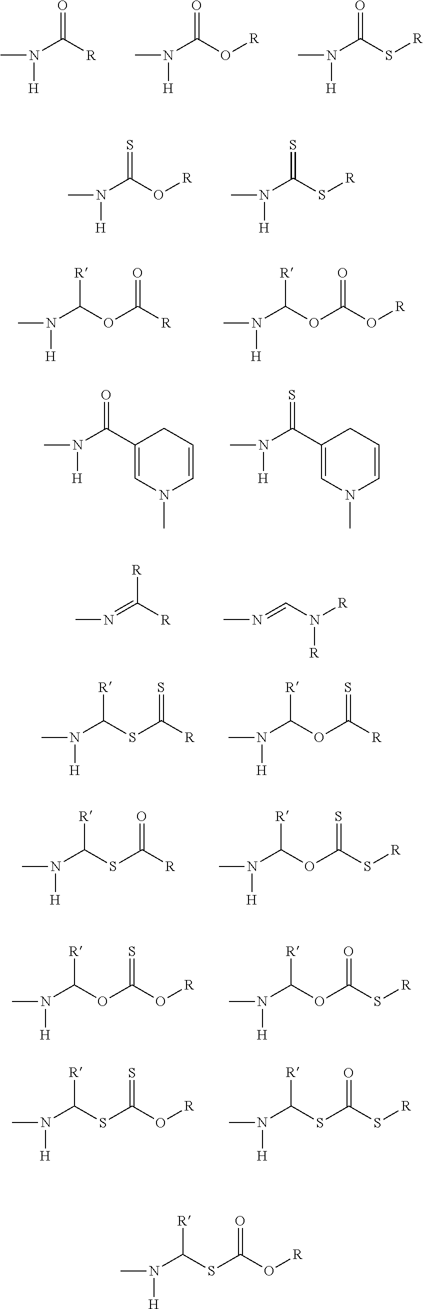

as well as sulfonamides and phosphonamides.

In certain instances, sites on any aromatic ring portions are susceptible to various metabolic reactions, therefore incorporation of appropriate substituents on the aromatic ring structures reduce, minimize, or eliminate this metabolic pathway.

### Metabolites

In some embodiments, compounds described herein are susceptible to various metabolic reactions. Therefore, in some embodiments, incorporation of appropriate substituents into the structure will reduce, minimize, or eliminate a metabolic pathway. In specific embodiments, the appropriate substituent to decrease or eliminate the susceptibility of an aromatic ring to metabolic reactions is, by way of example only, a halogen or an alkyl group.

In additional or further embodiments, the compounds described herein are metabolized upon administration to an organism in need to produce a metabolite that is then used to produce a desired effect, including a desired therapeutic effect.

### Pharmaceutical Compositions

In certain embodiments, the compound as described herein is administered as a pure chemical. In other embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)), the disclosure of which is hereby incorporated herein by reference in its entirety.

Accordingly, provided herein is a pharmaceutical composition comprising at least one compound described herein, or a stereoisomer, pharmaceutically acceptable salt, hydrate, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable carrier and a compound of Formula (A), Formula (I), or Formula (II), or a pharmaceutically acceptable salt or solvate thereof.

Another embodiment provides a pharmaceutical composition consisting essentially of a pharmaceutically acceptable carrier and a compound of Formula (A), Formula (I), or Formula (II), or a pharmaceutically acceptable salt or solvate thereof.

In certain embodiments, the compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, rectal, topical, buccal, parenteral (e.g., subcutaneous, intramuscular, intradermal, or intravenous), rectal, vaginal, or aerosol administration, although the most suitable form of administration in any given case will depend on the degree and severity of the condition being treated and on the nature of the particular compound being used. For example, disclosed compositions are formulated as a unit dose, and/or are formulated for oral or subcutaneous administration.

In some instances, exemplary pharmaceutical compositions are used in the form of a pharmaceutical preparation, for example, in solid, semisolid, or liquid form, which includes one or more of a disclosed compound, as an active ingredient, in admixture with an organic or inorganic carrier or excipient suitable for external, enteral, or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets in some instances, the principal active ingredient is mixed with a pharmaceutical carrier, e.g., conventional tableting ingredients such as corn starch, lactose, sucrose, sorbitol, talc, stearic acid, magnesium stearate, dicalcium phosphate, or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills, and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions also comprise buffering agents in some embodiments. Solid compositions of a similar type are also employed as fillers in soft and hard-filled gelatin capsules using such excipients as lactose or milk sugars, as well as high molecular weight polyethylene glycols and the like.

In some instances, a tablet is made by compression or molding, optionally with one or more accessory ingredients. Compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets are made by molding in a suitable machine a mixture of the subject composition moistened with an inert liquid diluent. Tablets, and other solid dosage forms, such as dragees, capsules, pills and granules, are optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms contain optionally inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, optionally contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which optionally contain antioxidants, buffers, bacteriostats, solutes which render the formulation isotonic with the blood of the intended recipient or suspending or thickening agents.

Examples of suitable aqueous and non-aqueous carriers employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. In some embodiments, proper fluidity is maintained, for example, by the use of coating materials, such as lecithin, by the maintenance of the required particle size in the case of dispersions, and by the use of surfactants

Also contemplated are enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure will recognize that it is not comprehensive and that there are other enteric materials that meet the objectives of the present disclosure.

In some embodiments, the doses of the composition comprising at least one compound as described herein differ, depending upon the patient's (e.g., human) condition, that is, stage of the disease, general health status, age, and other factors that a person skilled in the medical art will use to determine dose.

In some instances, pharmaceutical compositions are administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

In some embodiments, oral doses typically range from about 1.0 mg to about 1000 mg, one to four times, or more, per day.

### The Hippo Signaling Network

The Hippo signaling network (also known as the Salvador/Warts/Hippo (SWH) pathway) is a master regulator of cell proliferation, death, and differentiation. In some embodiments, the main function of the Hippo signaling pathway is to regulate negatively the transcriptional co-activators Yes-associated protein (YAP) and its paralogue, the transcriptional co-activator with PDZ-binding motif (TAZ; also known as WWTR1). The Hippo kinase cascade phosphorylates and inhibits YAP/TAZ by promoting its cytoplasmic retention and degradation, thereby inhibiting the growth promoting function regulated under the YAP/TAZ control. In an un-phosphorylated/de-phosphorylated state, YAP, also known as YAP1 or YAP65, together with TAZ, are transported into the nucleus where they interact with TEAD family of transcription factors to upregulate genes that promote proliferation and migration, and inhibit apoptosis. In some instances, unregulated upregulation of these genes involved in proliferation, migration, and anti-apoptosis leads to development of cancer. In some instances, overexpression of YAP/TAZ is associated with cancer.

Additional core members of the Hippo signaling pathway comprise the serine/threonine kinases MST1/2 (homologues of Hippo/Hpo in Drosophila), Lats1/2 (homologues of Warts/Wts), and their adaptor proteins Sav1 (homologue of Salvador/Sav) and Mob (MOBKL1A and MOBKL1B; homologues of Mats), respectively. In general, MST1/2 kinase complexes with the scaffold protein Sav1, which in turn phosphorylates and activates Lats1/2 kinase. Lats1/2 is also activated by the scaffold protein Mob. The activated Lats1/2 then phosphorylates and inactivates YAP or its paralog TAZ. The phosphorylation of YAP/TAZ leads to their nuclear export, retention within the cytoplasm, and degradation by the ubiquitin proteasome system.

In some instances, Lats1/2 phosphorylates YAP at the [HXRXXS] consensus motifs. YAP comprises five [HXRXXS] consensus motifs, wherein X denotes any amino acid residue. In some instances, Lats1/2 phosphorylates YAP at one or more of the consensus motifs. In some instances, Lats1/2 phosphorylates YAP at all five of the consensus motifs. In some instances, Lats1/2 phosphorylate at the S127 amino acid position. The phosphorylation of YAP S127 promotes 14-3-3 protein binding and results in cytoplasmic sequestration of YAP. Mutation of YAP at the S127 position thereby disrupts its interaction with 14-3-3 and subsequently promotes nuclear translocation.

Additional phosphorylation occurs at the S381 amino acid position in YAP. Phosphorylation of YAP at the S381 position and on the corresponding site in TAZ primes both proteins for further phosphorylation events by CK16/F in the degradation motif, which then signals for interaction with the β-TRCP E3 ubiquitin ligase, leading to polyubiquitination and degradation of YAP.

In some instances, Lats1/2 phosphorylates TAZ at the [HXRXXS] consensus motifs. TAZ comprises four [HXRXXS] consensus motifs, wherein X denotes any amino acid residues. In some instances, Lats1/2 phosphorylates TAZ at one or more of the consensus motifs. In some instances, Lats1/2 phosphorylates TAZ at all four of the consensus motifs. In some instances, Lats1/2 phosphorylate at the S89 amino acid position. The phosphorylation of TAZ S89 promotes 14-3-3 protein binding and results in cytoplasmic sequestration of TAZ. Mutation of TAZ at the S89 position thereby disrupts its interaction with 14-3-3 and subsequently promotes nuclear translocation.

In some embodiments, phosphorylated YAP/TAZ accumulates in the cytoplasm, and undergoes SCF^{β-TRCP}-mediated ubiquitination and subsequent proteasomal degradation. In some instances, the Skp, Cullin, F-box containing complex (SCF complex) is a multi-protein E3 ubiquitin ligase complex that comprises a F-box family member protein (e.g. Cdc4), Skp1, a bridging protein, and RBX1, which contains a small RING Finger domain which interacts with E2-ubiquitin conjugating enzyme. In some cases, the F-box family comprises more than 40 members, in which exemplary members include F-box/WD repeat-containing protein 1A (FBXW1A, βTrCP1, Fbxw1, hsSlimb, plkappaBalpha-E3 receptor subunit) and S-phase kinase-associated proteins 2 (SKP2). In some embodiments, the SCF complex (e.g. SCF^{βTrCP1}) interacts with an E1 ubiquitin-activating enzyme and an E2 ubiquitin-conjugating enzyme to catalyze the transfer of ubiquitin to the YAP/TAZ substrate. Exemplary E1 ubiquitin-activating enzymes include those encoded by the following genes: UBA1, UBA2, UBA3, UBA5, UBA5, UBA7, ATG7, NAE1, and SAE1. Exemplary E2 ubiquitin-conjugating enzymes include those encoded by the following genes: UBE2A, UBE2B, UBE2C, UBE2D1, UBE2D2, UBE2D3, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2M, UBE2N, UBE2O, UBE2Q1, UBE2Q2, UBE2R1, UBE2R2, UBE2S, UBE2T, UBE2U, UBE2V1, UBE2V2, UBE2Z, ATG2, BIRC5, and UFC1. In some embodiments, the ubiquitinated YAP/TAZ further undergoes the degradation process through the 26S proteasome.

In some embodiments, the Hippo pathway is regulated upstream by several different families of regulators. In some instances, the Hippo pathway is regulated by the G-protein and its coupled receptors, the Crumbs complex, regulators upstream of the MST kinases, and the adherens junction.

YAP/TAZ Interaction with TEAD

In some embodiments, un-phosphorylated and/or dephosphorylated YAP/TAZ accumulates in the nucleus. Within the nucleus, YAP/TAZ interacts with the TEAD family of transcription factors (e.g. TEAD1, TEAD2, TEAD3, or TEAD4) to activate genes involved in anti-apoptosis and proliferation, such as for example CTFG, Cyr61, and FGF1.

In some embodiments, the compounds disclosed herein modulate the interaction between YAP/TAZ and TEAD. In some embodiments, the compounds disclosed herein bind to TEAD, YAP, or TAZ and prevent the interaction between YAP/TAZ and TEAD.

### YAP/TAZ Regulation Mediated by G-Proteins GPCRs

In some embodiments, the Hippo pathway is regulated by the G protein-coupled receptor (GPCR) and G protein (also known as guanine nucleotide-binding proteins) family of proteins. G proteins are molecular switches that transmit extracellular stimuli into the cell through GPCRs. In some instances, there are two classes of G proteins: monomeric small GTPases and heterotrimeric G protein complexes. In some instances, the latter class of complexes comprise of alpha (G_{α}), beta (G_{β}), and gamma (G_{γ}) subunits. In some cases, there are several classes of G_{α} subunits: G_{q/11}α, G_{12/13}α, G_{i/o}α (G inhibitory, G other), and Gₛα (G stimulatory).

In some instances, Gᵢα (G inhibitory), Gₒα (G other), G_{q/11}α, and G_{12/13}α coupled GPCRs activate YAP/TAZ and promote nuclear translocation. In other instances, Gₛα (G stimulatory) coupled GPCRs suppress YAP/TAZ activity, leading to YAP/TAZ degradation.

In some cases, Gᵢα (G inhibitory), Gₒα (G other), G_{q/11}α, and G_{12/13}α coupled GPCRs activate YAP/TAZ through repression of Lats1/2 activities. In contrast, Gₛα, in some embodiments, induces Lats1/2 activity, thereby promoting YAP/TAZ degradation.

### G_{q} Family

G_{q}α (also known as G_{q/11} protein), participates in the inositol trisphosphate (IP₃) signal transduction pathway and calcium (Ca²⁺) release from intracellular storage through the activation of phospholipase C (PLC). The activated PLC hydrolyzes phosphatidylinositol 4,5-bisphosphate (PIP₂) to diacyl glycerol (DAG) and IP₃. In some instances, IP₃ then diffuses through the cytoplasm into the ER or the sarcoplasmic reticulum (SR) in the case of muscle cells, and then binds to inositol trisphosphate receptor (InsP3R), which is a Ca²⁺ channel. In some cases, the binding triggers the opening of the Ca²⁺ channel, and thereby increases the release of Ca²⁺ into the cytoplasm.

In some embodiments, the GPCRs that interact with G_{q}α include, but are not limited to, 5-hydroxytryptamine receptor (5-HT receptor) types 5-HT₂ and 5-HT₃; alpha-1 adrenergic receptor; vasopressin type 1 receptors 1A and 1B; angiotensin II receptor type 1; calcitonin receptor; histamine H1 receptor; metabotropic glutamate receptor, group I; muscarinic receptors M₁, M₃, and M₅; and trace amine-associated receptor 1.

In some instances, there are several types of G_{q}α. G_{q}, G_{q/11}, G_{q/14}, and G_{q/15}. The G_{q} protein is encoded by GNAQ. G_{q/11} is encoded by GNA11. G_{q/14} is encoded by GNA14. G_{q/15} is encoded by GNA15.

In some instances, mutations or modifications of the G_{q}α genes have been associated with cancer. Indeed, studies have shown that mutations in G_{q}α promote uveal melanoma (UM) tumorigenesis. In some instances, about 80% of UM cases have been detected to contain a mutation in GNAQ and/or GNA11.

In some instances, mutations or modifications of the G_{q}α genes have been associated with congenital diseases. In some instances, mutations of G_{q}α have been observed in congenital diseases such as Port-Wine Stain and/or Sturge-Weber Syndrome. In some instances, about 92% of Port-Wine stain cases harbors a mutation in GNAQ. In some instances, about 88% of Sturge-Weber Syndrome harbors a mutation in GNAQ.

### G_{12/13} Family

G_{12/13}α modulates actin cytoskeletal remodeling in cells and regulates cell processes through guanine nucleotide exchange factors (GEFs). GEFs participate in the activation of small GTPases which acts as molecular switches in a variety of intracellular signaling pathways. Examples of small GTPases include the Ras-related GTPase superfamily (e.g. Rho family such as Cdc42), which is involved in cell differentiation, proliferation, cytoskeletal organization, vesicle trafficking, and nuclear transport.

In some embodiments, the GPCRs that interact with G_{12/13}α include, but are not limited to, purinergic receptors (e.g. P2Y₁, P2Y₂, P2Y₄, P2Y₆); muscarinic acetylcholine receptors M1 and M3; receptors for thrombin [protease-activated receptor (PAR)-1, PAR-2]; thromboxane (TXA2); sphingosine 1-phosphate (e.g. S1P₂, S1P₃, S1P₄ and S1P₅); lysophosphatidic acid (e.g. LPA₁, LPA₂, LPA₃); angiotensin II (AT1); serotonin (5-HT_{2c} and 5-HT₄); somatostatin (sst₅); endothelin (ET_{A} and ET_{B}); cholecystokinin (CCK₁); V₁ₐ vasopressin receptors; D₅ dopamine receptors; fMLP formyl peptide receptors; GAL₂ galanin receptors; EP₃ prostanoid receptors; A₁ adenosine receptors; α₁ adrenergic receptors; BB₂ bombesin receptors; B₂ bradykinin receptors; calcium-sensing receptors; KSHV-ORF74 chemokine receptors; NK₁ tachykinin receptors; and thyroid-stimulating hormone (TSH) receptors.

In some instances, G_{12/13}α is further subdivided into G₁₂ and G₁₃ types which are encoded by GNA12 and GNA13, respectively.

### G_{i/o} Family

G_{i/o}α (G inhibitory, G other) (also known as Gᵢ/G₀ or Gᵢ protein) suppresses the production of 3′,5′-cyclic AMP (cAMP) from adenosine triphosphate (ATP) through an inhibition of adenylate cyclase activity, which converts ATP to cAMP.

In some embodiments, the GPCRs that interact with Gᵢα include, but are not limited to, 5-hydroxytryptamine receptor (5-HT receptor) types 5-HT₁ and 5-HT₅; muscarinic acetylcholine receptors such as M₂ and M₄; adenosine receptors such as A₁ and A₃; adrenergic receptors such as α_{2A}, α_{2B}, and α_{2C}; apelin receptors; calcium-sensing receptor; cannabinoid receptors CB1 and CB2; chemokine CXCR4 receptor; dopamines D₂, D₃, and D₄; GABA_{B} receptor; glutamate receptors such as metabotropic glutamate receptor 2 (mGluR2), metabotropic glutamate receptor 3 (mGluR3), metabotropic glutamate receptor 4 (mGluR4), metabotropic glutamate receptor 6 (mGluR6), metabotropic glutamate receptor 7 (mGluR7), and metabotropic glutamate receptor 8 (mGluR8); histamine receptors such as H₃ and H₄ receptors; melatonin receptors such as melatonin receptor type 1 (MT1), melatonin receptor type 2 (MT2), and melatonin receptor type 3 (MT3); niacin receptors such as NIACR1 and NIACR2; opioid receptors such as δ, κ, μ, and nociceptin receptors; prostaglandin receptors such as prostaglandin E receptor 1 (EP₁), prostaglandin E receptor 3 (EP₃), prostaglandin F receptor (FP), and thromboxane receptor (TP); somatostatin receptors sst1, sst2, sst3, sst4, and sst5; and trace amine-associated receptor 8.

In some instances, there are several types of Gᵢα: Gᵢα1, Gᵢα2, Gᵢα3, Gᵢα4, Gₒα, Gₜ, G_{gust}, and G_{z}. Gᵢα1 is encoded by GNAI1. Gᵢα2 is encoded by GNAI2. Gᵢα3 is encoded by GNAI3. Gₒα, the aₒ subunit, is encoded by GNAO1. Gₜ is encoded by GNAT1 and GNAT2. G_{gust} is encoded by GNAT3. G_{z} is encoded by GNAZ.

### Gₛ Family

Gₛα (also known as G stimulatory, Gₛ alpha subunit, or Gₛ protein) activates the cAMP-dependent pathway through the activation of adenylate cyclase, which convers adenosine triphosphate (ATP) to 3′,5′-cyclic AMP (cAMP) and pyrophosphate. In some embodiments, the GPCRs that interact with Gₛα include, but are not limited to, 5-hydroxytryptamine receptor (5-HT receptor) types 5-HT₄, 5-HT₆, and 5-HT₇; adrenocorticotropic hormone receptor (ACTH receptor) (also known as melanocortin receptor 2 or MC2R); adenosine receptor types A₂ₐ and A_{2b}; arginine vasopressin receptor 2 (AVPR2); β-adrenergic receptors β₁, β₂, and β₃; calcitonin receptor; calcitonin gene-related peptide receptor; corticotropin-releasing hormone receptor; dopamine receptor D₁-like family receptors such as D₁ and D₅; follicle-stimulating hormone receptor (FSH-receptor); gastric inhibitory polypeptide receptor; glucagon receptor; histamine H₂ receptor; luteinizing hormone/choriogonadotropin receptor; melanocortin receptors such as MC1R, MC2R, MC3R, MC4R, and MC5R; parathyroid hormone receptor 1; prostaglandin receptor types D₂ and I₂; secretin receptor; thyrotropin receptor; trace amine-associated receptor 1; and box jellyfish opsin.

In some instances, there are two types of Gₛα: Gₛ and G_{olf}. Gₛ is encoded by GNAS. G_{olf} is encoded by GNAL.

### Additional Regulators of the Hippo Signaling Network

In some embodiments, the additional regulator of the Hippo signaling pathway is the Crumbs (Crb) complex. The Crumbs complex is a key regulator of cell polarity and cell shape. In some instances, the Crumbs complex comprises transmembrane CRB proteins which assemble multi-protein complexes that function in cell polarity. In some instances, CRB complexes recruit members of the Angiomotin (AMOT) family of adaptor proteins that interact with the Hippo pathway components. In some instances, studies have shown that AMOT directly binds to YAP, promotes YAP phosphorylation, and inhibits its nuclear localization.

In some instances, the additional regulator of the Hippo signaling pathway comprises regulators of the MST kinase family. MST kinases monitor actin cytoskeletal integrity. In some instances, the regulators include TAO kinases and cell polarity kinase PAR-1.

In some instances, the additional regulator of the Hippo signaling pathway comprises molecules of the adherens junction. In some instances, E-Cadherin (E-cad) suppresses YAP nuclear localization and activity through regulating MST activity. In some embodiments, E-cad-associated protein α-catenin regulates YAP through sequestering YAP/14-3-3 complexes in the cytoplasm. In other instances, Ajuba protein family members interact with Lats1/2 kinase activity, thereby preventing inactivation of YAP/TAZ.

In some embodiments, additional proteins that interact with YAP/TAZ either directly or indirectly include, but are not limited to, Merlin, protocadherin Fat 1, MASK1/2, HIPK2, PTPN14, RASSF, PP2A, Salt-inducible kinases (SIKs), Scribble (SCRIB), the Scribble associated proteins Discs large (Dlg), KIBRA, PTPN14, NPHP3, LKB1, Ajuba, and ZO1/2.

In some embodiments, the compounds described herein are inhibitors of transcriptional coactivator with PDZ binding motif/Yes-associated protein transcriptional coactivator (TAZ/YAP). In some embodiments, the compounds described herein increase the phosphorylation of transcriptional coactivator with PDZ binding motif/Yes-associated protein transcriptional coactivator (TAZ/YAP) or decrease the dephosphorylation of transcriptional coactivator with PDZ binding motif/Yes-associated protein transcriptional coactivator (TAZ/YAP). In some embodiments, the compounds increase the ubiquitination of transcriptional coactivator with PDZ binding motif/Yes-associated protein transcriptional coactivator (TAZ/YAP) or decrease the deubiquitination of transcriptional coactivator with PDZ binding motif/Yes-associated protein transcriptional coactivator (TAZ/YAP).

In some embodiments, the compounds disclosed herein are inhibitors of one or more of the proteins encompassed by, or related to, the Hippo pathway. In some instances, the one or more proteins comprise a protein described above. In some embodiments, an inhibitor of the Hippo pathway is an inhibitor of a G-protein and/or its coupled GPCR. In some embodiments, an inhibitor of the Hippo pathway is an inhibitor of a G-protein. In some embodiments, an inhibitor of the Hippo pathway is an inhibitor of the G_{q}a family proteins such as G_{q}, G_{q/11}, G_{q/14}, and G_{q/15}; the G_{12/13}α family of proteins such as G₁₂ and G₁₃; or the Gᵢα family of proteins such as Gᵢα1, Gᵢα2, Gᵢα3, Gᵢα4, Gₒα, Gₜ, G_{gust}, and G_{z}. In some embodiments, an inhibitor of the Hippo pathway is an inhibitor of G_{q}. In some embodiments, an inhibitor of the Hippo pathway is an inhibitor of G_{q/11}. In some embodiments, an inhibitor of the Hippo pathway is an inhibitor of G_{q/14}. In some embodiments, an inhibitor of the Hippo pathway is an inhibitor of G_{q/15}. In some embodiments, an inhibitor of the Hippo pathway is an inhibitor of G₁₂. In some embodiments, an inhibitor of the Hippo pathway is an inhibitor of G₁₃. In some embodiments, an inhibitor of the Hippo pathway is an inhibitor of Gᵢα1. In some embodiments, an inhibitor of the Hippo pathway is an inhibitor of Gᵢα2. In some embodiments, an inhibitor of the Hippo pathway is an inhibitor of Gᵢα3. In some embodiments, an inhibitor of the Hippo pathway is an inhibitor of Gᵢα4. In some embodiments, an inhibitor of the Hippo pathway is an inhibitor of Gₒα. In some embodiments, an inhibitor of the Hippo pathway is an inhibitor of Gₜ. In some embodiments, an inhibitor of the Hippo pathway is an inhibitor of G_{gust}. In some embodiments, an inhibitor of the Hippo pathway is an inhibitor of G_{z}.

In some embodiments, an inhibitor of the Hippo pathway is an inhibitor of a core protein of the Hippo pathway. In some embodiments, an inhibitor of the Hippo pathway is an inhibitor of Sav1. In some embodiments, an inhibitor of the Hippo pathway is an inhibitor of Mob. In some embodiments, an inhibitor of the Hippo pathway is an inhibitor of YAP. In some embodiments, an inhibitor of the Hippo pathway is an inhibitor of TAZ. In some embodiments, an inhibitor of the Hippo pathway is an inhibitor of TEAD.

In some embodiments, an inhibitor of the Hippo pathway is an inhibitor of a protein associated with the ubiquitination and proteasomal degradation pathway. In some embodiments, an inhibitor of the Hippo pathway is an inhibitor of a proteasomal degradation pathway protein (e.g. 26S proteasome).

In some embodiments, an inhibitor of the Hippo pathway is an inhibitor of a protein of the Ras superfamily of proteins. In some embodiments, an inhibitor of the Hippo pathway is an inhibitor of a protein of the Rho family of proteins. In some embodiments, an inhibitor of the Hippo pathway is an inhibitor of Cdc42.

Cdc42 is a member of the Ras superfamily of small GTPases. Specifically, Cdc42 belongs to the Rho family of GTPases, in which the family members participate in diverse and critical cellular processes such as gene transcription, cell-cell adhesion, and cell cycle progression. Cdc42 is involved in cell growth and polarity, and in some instances, Cdc42 is activated by guanine nucleotide exchange factors (GEFs). In some cases, an inhibitor of Cdc42 is a compound disclosed herein.

In some embodiments, an inhibitor of the Hippo pathway is an inhibitor of a deubiquitinating enzyme. In some embodiments, an inhibitor of the Hippo pathway is an inhibitor of a cysteine protease or a metalloprotease. In some embodiments, an inhibitor of the Hippo pathway is an inhibitor of an ubiquitin-specific protease. USP47 is a member of the ubiquitin-specific protease (USP/UBP) superfamily of cysteine proteases. In some embodiments, the compounds disclosed herein are inhibitors of USP47.

Further embodiments provided herein include combinations of one or more of the particular embodiments set forth above.

### Diseases

### Cancer

In some embodiments, the compounds disclosed herein are useful for treating cancer. In some embodiments, disclosed herein is a method for treating a cancer in a subject in need thereof comprising administering a therapeutically effective amount of a compound disclosed herein or a pharmaceutically acceptable salt or solvate thereof. In some embodiments, disclosed herein is a compound for use in treating a cancer in a subject in need thereof comprising administering to the subject in need thereof a therapeutically effective amount of a compound disclosed herein or a pharmaceutically acceptable salt or solvate thereof. In some embodiments, the cancer is mediated by activation of transcriptional coactivator with PDZ binding motif/Yes-associated protein transcription coactivator (TAZ/YAP). In some embodiments, the cancer is mediated by modulation of the interaction of YAP/TAZ with TEAD. In some embodiments, the cancer is characterized by a mutant Gα-protein. In some embodiments, the mutant Gα-protein is selected from G12, G13, Gq, G11, Gi, Go, and Gs. In some embodiments, the mutant Gα-protein is G12. In some embodiments, the mutant Gα-protein is G13. In some embodiments, the mutant Gα-protein is Gq. In some embodiments, the mutant Gα-protein is G11. In some embodiments, the mutant Gα-protein is Gi. In some embodiments, the mutant Gα-protein is Go. In some embodiments, the mutant Gα-protein is Gs.

In some embodiments, the cancer is a solid tumor. In some instances, the cancer is a hematologic malignancy. In some instances, the solid tumor is a sarcoma or carcinoma. In some instances, the solid tumor is a sarcoma. In some instances, the solid tumor is a carcinoma.

Exemplary sarcoma includes, but is not limited to, alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epithelioid cell differentiation, osteosarcoma, parosteal osteosarcoma, neoplasm with perivascular epithelioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, and telangiectatic osteosarcoma.

Exemplary carcinoma includes, but is not limited to, adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, and vulvar cancer. In some instances, the liver cancer is primary liver cancer.

In some instances, the cancer is selected from uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, and meningioma. In some cases, the cancer is uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, or meningioma. In some cases, the cancer is uveal melanoma, mesothelioma, esophageal cancer, or liver cancer. In some cases, the cancer is uveal melanoma. In some cases, the cancer is mesothelioma. In some cases, the cancer is esophageal cancer. In some cases, the cancer is liver cancer. In some cases, the cancer is primary liver cancer.

In some instances, the cancer is a hematologic malignancy. In some embodiments, a hematologic malignancy is a leukemia, a lymphoma, a myeloma, a non-Hodgkin's lymphoma, a Hodgkin's lymphoma, a T-cell malignancy, or a B-cell malignancy. In some instances, a hematologic malignancy is a T-cell malignancy. Exemplary T-cell malignancy includes, but is not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, and treatment-related T-cell lymphomas.

In some instances, a hematologic malignancy is a B-cell malignancy. Exemplary B-cell malignancy includes, but is not limited to, chronic lymphocytic leukemia (CLL), small lymphocytic lymphoma (SLL), high risk CLL, and a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some instances, the cancer is a relapsed or refractory cancer. In some embodiments, the relapsed or refractory cancer is a relapsed or refractory solid tumor. In some embodiments, the relapsed or refractory solid tumor is a relapsed or refractory sarcoma or a relapsed or refractory carcinoma. In some embodiments, the relapsed or refractory carcinoma includes adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, and vulvar cancer.

In some instances, the relapsed or refractory cancer is selected from relapsed or refractory uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, and meningioma. In some cases, the relapsed or refractory cancer is relapsed or refractory uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, or meningioma. In some cases, the relapsed or refractory cancer is relapsed or refractory uveal melanoma, mesothelioma, esophageal cancer, or liver cancer. In some cases, the relapsed or refractory cancer is relapsed or refractory uveal melanoma. In some cases, the relapsed or refractory cancer is relapsed or refractory mesothelioma. In some cases, the relapsed or refractory cancer is relapsed or refractory esophageal cancer. In some cases, the relapsed or refractory cancer is relapsed or refractory liver cancer. In some cases, the relapsed or refractory cancer is relapsed or refractory primary liver cancer.

In some instances, the relapsed or refractory cancer is a relapsed or refractory hematologic malignancy. In some embodiments, a relapsed or refractory hematologic malignancy is a relapsed or refractory leukemia, a relapsed or refractory lymphoma, a relapsed or refractory myeloma, a relapsed or refractory non-Hodgkin's lymphoma, a relapsed or refractory Hodgkin's lymphoma, a relapsed or refractory T-cell malignancy, or a relapsed or refractory B-cell malignancy. In some instances, a relapsed or refractory hematologic malignancy is a relapsed or refractory T-cell malignancy. In some instances, a relapsed or refractory hematologic malignancy is a relapsed or refractory B-cell malignancy, such as for example, chronic lymphocytic leukemia (CLL), small lymphocytic lymphoma (SLL), high risk CLL, or a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some instances, the cancer is a metastasized cancer. In some instances, the metastasized cancer is a metastasized solid tumor. In some instances, the metastasized solid tumor is a metastasized sarcoma or a metastasized carcinoma. In some embodiments, the metastasized carcinoma includes adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, and vulvar cancer.

In some instances, the metastasized cancer is selected from metastasized uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, and meningioma. In some cases, the metastasized cancer is metastasized uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, or meningioma. In some cases, the metastasized cancer is metastasized uveal melanoma, mesothelioma, esophageal cancer, or liver cancer. In some cases, the metastasized cancer is metastasized uveal melanoma. In some cases, the metastasized cancer is metastasized mesothelioma. In some cases, the metastasized cancer is metastasized esophageal cancer. In some cases, the metastasized cancer is metastasized liver cancer. In some cases, the metastasized cancer is metastasized primary liver cancer.

In some instances, the metastasized cancer is a metastasized hematologic malignancy. In some embodiments, the metastasized hematologic malignancy is a metastasized leukemia, a metastasized lymphoma, a metastasized myeloma, a metastasized non-Hodgkin's lymphoma, a metastasized Hodgkin's lymphoma, a metastasized T-cell malignancy, or a metastasized B-cell malignancy. In some instances, a metastasized hematologic malignancy is a metastasized T-cell malignancy. In some instances, a metastasized hematologic malignancy is a metastasized B-cell malignancy, such as for example, chronic lymphocytic leukemia (CLL), small lymphocytic lymphoma (SLL), high risk CLL, or a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

### Congenital Diseases

In some embodiments, the compounds disclosed herein are useful for treating a congenital disease. In some embodiments, the congenital disease is mediated by activation of transcriptional coactivator with PDZ binding motif/Yes-associated protein transcription coactivator (TAZ/YAP). In some embodiments, the congenital disease is characterized by a mutant Gα-protein. In some embodiments, the mutant Gα-protein is selected from G12, G13, Gq, G11, Gi, Go, and Gs. In some embodiments, the mutant Gα-protein is G12. In some embodiments, the mutant Gα-protein is G13. In some embodiments, the mutant Gα-protein is Gq. In some embodiments, the mutant Gα-protein is G11. In some embodiments, the mutant Gα-protein is Gi. In some embodiments, the mutant Gα-protein is Go. In some embodiments, the mutant Gα-protein is Gs.

In some embodiments, the congenital disease is the result of a genetic abnormality, an intrauterine environment, errors related to morphogenesis, infection, epigenetic modifications on a parental germline, or a chromosomal abnormality. Exemplary congenital diseases include, but are not limited to, Sturge-Weber Syndrome, Port-Wine stain, Holt-Oram syndrome, abdominal wall defects, Becker muscular dystrophy (BMD), biotinidase deficiency, Charcot-Marie-Tooth (CMT), cleft lip, cleft palate, congenital adrenal hyperplasia, congenital heart defects, congenital hypothyroidism, congenital muscular dystrophy, cystic fibrosis, Down syndrome, Duchenne muscular dystrophy, Fragile X syndrome, Friedreich's ataxia, galactosemia, hemoglobinopathies, Krabbe disease, limb-girdle muscular dystrophy, medium chain acyl-CoA dehydrogenase deficiency, myasthenia gravis, neural tube defects, phenylketonuria, Pompe disease, severe combined immunodeficiency (SCID), Stickler syndrome (or hereditary progressive arthro-ophthalmopathy), spinal muscular atrophy, and trisomy 18. In some embodiments, the congenital disease is Sturge-Weber Syndrome or Port-Wine stain. In some embodiments, the congenital disease is Sturge-Weber Syndrome. In some embodiments, the congenital disease is Port-Wine stain.

### EXAMPLES

These examples are provided for illustrative purposes only and not to limit the scope of the claims provided herein.

### List of Abbreviations

As used above, and throughout the disclosure, the following abbreviations, unless otherwise indicated, shall be understood to have the following meanings:
ACN or MeCN acetonitrile
Ac acetyl
BOC or Boc tert-butyl carbamate
t-Bu tert-butyl
° C. degrees Celsius
DBA or dba dibenzylideneacetone
DCE dichloroethane (ClCH₂CH₂Cl)
DCM dichloromethane (CH₂Cl₂)
DIPEA or DIEA diisopropylethylamine
DMF dimethylformamide
DMSO dimethylsulfoxide
EA or EtOAc ethyl acetate
Et ethyl
EtOH ethanol
g gram(s)
h, hr, hrs hour(s)
HPLC high performance liquid chromatography
Hz hertz
LCMS liquid chromatography mass spectrometry
m/z mass-to-charge ratio
M molar
Me methyl
MeOH methanol
mg milligram(s)
MHz megahertz
umol micromole(s)
uL microliter(s)
mL milliliter(s)
mmol millimole(s)
MS mass spectroscopy
NMR nuclear magnetic resonance
PE petroleum ether
Ph phenyl
prep-HPLC preparative high pressure liquid chromatography
prep-TLC preparative thin layer chromatography
Py pyridine
RT retention time
TEA triethylamine
TFA trifluoroacetic acid
THF tetrahydrofuran
TLC thin layer chromatography

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times were approximate and were not optimized. Column chromatography and thin layer chromatography (TLC) were performed on silica gel unless otherwise noted.

In some embodiments, compounds are prepared by following procedures described in international application no. PCT/US2020/028363, and/or as described herein.

### Example A: 5-(4-(Trifluoromethyl)phenoxy)-2-naphthoic acid

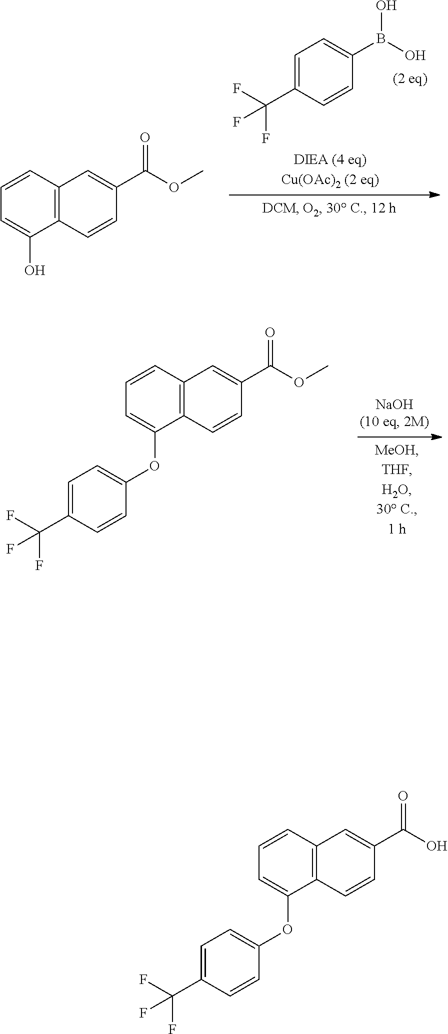 [SMILES: COC(=O)c1ccc2c(O)cccc2c1 ; OB(O)c1ccc(C(F)(F)F)cc1 ; COC(=O)c1ccc2c(Oc3ccc(C(F)(F)F)cc3)cccc2c1 ; O=C(O)c1ccc2c(Oc3ccc(C(F)(F)F)cc3)cccc2c1]

methyl 5-[4-(trifluoromethyl)phenoxy]naphthalene-2-carboxylate: to a solution of methyl 5-hydroxy-2-naphthoate (3.6 g, 17.80 mmol, 1 eq) and (4-(trifluoromethyl)phenyl)boronic acid (6.76 g, 35.61 mmol, 2 eq) in DCM (120 mL) were added DIEA (9.20 g, 71.21 mmol, 12.40 mL, 4 eq) and Cu(OAc)₂ (6.47 g, 35.61 mmol, 2 eq) under O₂. The mixture was degassed under vacuum and purged with O₂ 3 times. The mixture was stirred under O₂ (15 psi) at 30° C. for 12 hours. The mixture was filtered. The filtrate was diluted with H₂O (250 mL), extracted with EA (500 mL*3). The combined organic layers were washed with brine (300 mL), dried over Na₂SO₄, filtered and concentrated under reduced pressure to give a residue. The residue was purified by flash silica gel chromatography to give methyl 5-[4-(trifluoromethyl)phenoxy]naphthalene-2-carboxylate (2.5 g, 7.22 mmol, 20.3% yield) and 1 (1.5 g, 7.42 mmol, 20.8% yield).

5-[4-(trifluoromethyl)phenoxy]naphthalene-2-carboxylic acid: to a mixture of methyl 5-[4-(trifluoromethyl)phenoxy]naphthalene-2-carboxylate (200 mg, 0.58 mmol, 1 eq) in MeOH (1.5 mL), THF (0.5 mL) and H₂O (0.5 mL) was added NaOH (2 M, 2.89 mL, 10 eq). The mixture was stirred at 30° C. for 1 h. The mixture was concentrated. The residue was diluted with H₂O (20 mL) and adjusted pH=6-7 with 1N HCl. The mixture was extracted with EA (40 mL*3). The combined organic layers were washed with brine (30 mL), dried over Na₂SO₄, filtered and concentrated under reduced pressure to give 5-[4-(trifluoromethyl)phenoxy]naphthalene-2-carboxylic acid (220 mg, crude).

### Example 1: (3-aminoazetidin-1-yl)(5-(4-(trifluoromethyl)phenoxy)naphthalen-2-yl)methanone (Compound 1A) and N-(1-(5-(4-(Trifluoromethyl)phenoxy)-2-naphthoyl)azetidin-3-yl)acetamide (Compound 1)

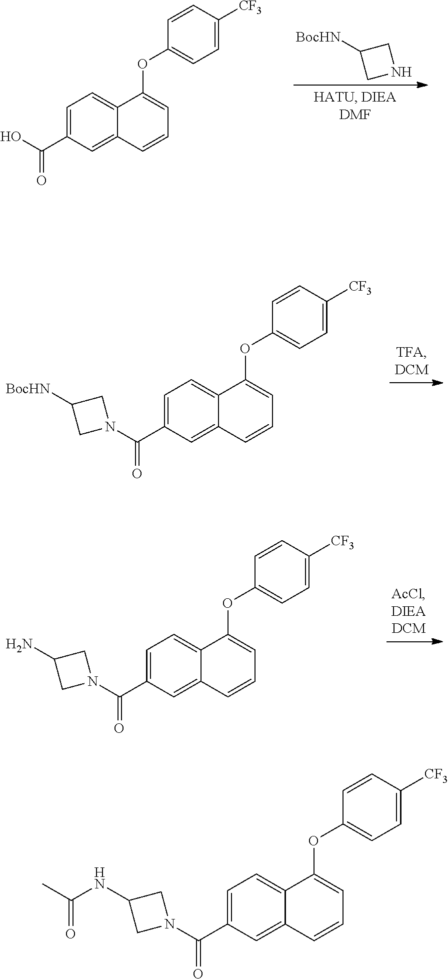 [SMILES: O=C(O)c1ccc2c(Oc3ccc(C(F)(F)F)cc3)cccc2c1 ; CC(C)(C)OC(=O)NC1CNC1 ; CC(C)(C)OC(=O)NC1CN(C(=O)c2ccc3c(Oc4ccc(C(F)(F)F)cc4)cccc3c2)C1 ; NC1CN(C(=O)c2ccc3c(Oc4ccc(C(F)(F)F)cc4)cccc3c2)C1 ; CC(=O)NC1CN(C(=O)c2ccc3c(Oc4ccc(C(F)(F)F)cc4)cccc3c2)C1]

tert-butyl (1-(5-(4-(trifluoromethyl)phenoxy)-2-naphthoyl)azetidin-3-yl)carbamate: 5-(4-(Trifluoromethyl)phenoxy)-2-naphthoic acid (66 mg, 0.2 mmol, 1 eq.), tert-butyl azetidin-3-ylcarbamate HCl (46 mg, 0.22 mmol, 1.1 eq.), HATU (91 mg, 0.24 mmol, 1.2 eq.), DIEA (87 μL, 0.5 mmol, 2.5 eq.), and DMF (1 mL, 0.2 M) were stirred at 23° C. until LCMS indicated complete conversion of the starting material, 2 hr. The reaction mixture was diluted with H₂O, and the resulting precipitate was filtered, rinsed with H₂O, and dried to give tert-butyl (1-(5-(4-(trifluoromethyl)phenoxy)-2-naphthoyl)azetidin-3-yl)carbamate (86 mg, 0.18 mmol, 88% yield).

(3-Aminoazetidin-1-yl)(5-(4-(trifluoromethyl)phenoxy)naphthalen-2-yl)methanone: tert-butyl (1-(5-(4-(trifluoromethyl)phenoxy)-2-naphthoyl)azetidin-3-yl)carbamate (49 mg, 0.1 mmol, 1 eq.) was dissolved in DCM (0.8 mL), and TFA (0.2 mL) was added carefully. The reaction mixture was stirred at 23° C. until LCMS indicated complete conversion to the desired product, 1 hr. Upon completion, mixture was concentrated, and the residue rinsed with heptane to give the desired product, (3-aminoazetidin-1-yl)(5-(4-(trifluoromethyl)phenoxy)naphthalen-2-yl)methanone, as the TFA salt (50 mg, 0.1 mmol, 100% yield). LCMS: [M+H]⁺: 387.1. ¹H NMR: (400 MHz, DMSO-d6) δ ppm 3.91 (br s, 3H) 4.13 (br s, 2H) 4.21-4.41 (m, 2H) 4.72 (br t, J=7.78 Hz, 1H) 7.18 (d, J=8.53 Hz, 2H) 7.35 (d, J=7.35 Hz, 1H) 7.65 (t, J=8.03 Hz, 1H) 7.72-7.79 (m, 3H) 8.04 (d, J=8.19 Hz, 2H) 8.33 (s, 1H) 8.51 (br s, 3H).

N-(1-(5-(4-(Trifluoromethyl)phenoxy)-2-naphthoyl)azetidin-3-yl)acetamide: (3-Aminoazetidin-1-yl)(5-(4-(trifluoromethyl)phenoxy)naphthalen-2-yl)methanone, TFA salt (10 mg, 0.02 mmol, 1 eq.) was dissolved in DCM (1 mL) and cooled to 0° C. DIEA (7 μL, 0.04 mmol, 2 eq.) was carefully added, followed by AcCl (2 μL, 0.026 mmol, 1.3 eq.). The reaction mixture was warmed to 23° C. and stirred 2 hr. Upon completion, the mixture was diluted with DCM, washed with sat. NaHCO₃, H₂O, and brine. The organic layer was dried with Na₂SO₄, concentrated, and purified by semi-prep HPLC (Luna C18, 5 μm, 100 Å, 250×10 mm, ACN+0.1% TFA:H₂O+0.1% TFA, gradient) to give the desired product as the TFA salt (6 mg, 55% yield). LCMS: [M+H]+: 429.0. ¹H NMR: (400 MHz, DMSO-d6) δ ppm 1.84 (s, 3H) 3.85-4.03 (m, 1H) 4.18 (br d, J=8.13 Hz, 1H) 4.35 (br t, J=8.94 Hz, 1H) 4.46-4.70 (m, 2H) 7.18 (d, J=8.50 Hz, 2H) 7.31-7.37 (m, 1H) 7.64 (t, J=7.94 Hz, 1H) 7.71-7.80 (m, 3H) 8.02 (dd, J=8.51, 4.00 Hz, 2H) 8.31-8.35 (m, 1H) 8.58 (br d, J=6.63 Hz, 1H).

### Example 2: Piperazin-1-yl(5-(4-(trifluoromethyl)phenoxy)naphthalen-2-yl)methanone (Compound 2)

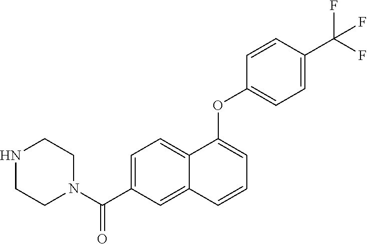 [SMILES: O=C(c1ccc2c(Oc3ccc(C(F)(F)F)cc3)cccc2c1)N1CCNCC1]

The title compound was synthesized following the procedure outlined for (3-aminoazetidin-1-yl)(5-(4-(trifluoromethyl)phenoxy)naphthalen-2-yl)methanone. LCMS: [M+H]⁺: 401.1 ¹H NMR: (400 MHz, DMSO-d6) δ ppm 3.20 (br s, 4H) 7.18 (d, J=8.53 Hz, 2H) 7.34 (d, J=7.03 Hz, 1H) 7.59-7.68 (m, 2H) 7.76 (d, J=8.78 Hz, 2H) 7.96 (d, J=8.53 Hz, 1H) 8.03 (d, J=8.78 Hz, 1H) 8.18 (s, 1H) 8.99 (br s, 2H).

### Example 3: (3-Aminopyrrolidin-1-yl)(5-(4-(trifluoromethyl)phenoxy)naphthalen-2-yl)methanone (Compound 3)

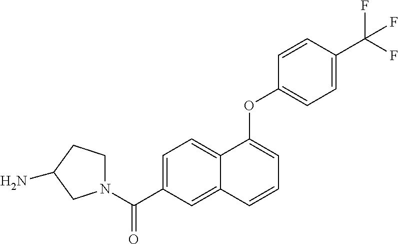 [SMILES: NC1CCN(C(=O)c2ccc3c(Oc4ccc(C(F)(F)F)cc4)cccc3c2)C1]

The title compound was synthesized following the procedure outlined for 3-aminoazetidin-1-yl)(5-(4-(trifluoromethyl)phenoxy)naphthalen-2-yl)methanone. LCMS: [M+H]⁺: 401.1. ¹H NMR: (400 MHz, DMSO-d6) δ ppm 2.01 (br s, 1H) 2.19-2.33 (m, 1H) 3.59 (br s, 2H) 3.66 (br d, J=9.79 Hz, 2H) 3.72-3.87 (m, 2H) 3.92 (br s, 1H) 7.18 (br d, J=8.28 Hz, 2H) 7.35 (br t, J=6.90 Hz, 1H) 7.62-7.70 (m, 2H) 7.76 (d, J=8.53 Hz, 2H) 7.91-8.07 (m, 4H) 8.10 (br s, 2H) 8.23 (s, 1H).

### Example 4: (4-Aminopiperidin-1-yl)(5-(4-(trifluoromethyl)phenoxy)naphthalen-2-yl)methanone (Compound 4)

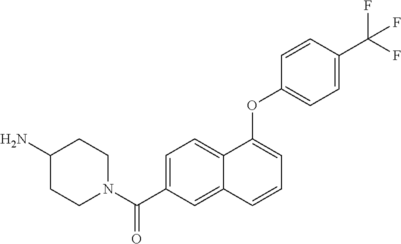 [SMILES: NC1CCN(C(=O)c2ccc3c(Oc4ccc(C(F)(F)F)cc4)cccc3c2)CC1]

The title compound was synthesized following the procedure outlined for 3-aminoazetidin-1-yl)(5-(4-(trifluoromethyl)phenoxy)naphthalen-2-yl)methanone. LCMS: [M+H]⁺: 415.0. ¹H NMR: (400 MHz, DMSO-d6) δ ppm 1.47 (br s, 2H) 1.89 (br s, 1H) 2.02 (br s, 1H) 2.92 (br d, J=15.63 Hz, 1H) 3.17 (br s, 2H) 3.70 (br s, 1H) 4.54 (br s, 1H) 7.18 (m, J=8.50 Hz, 2H) 7.33 (dd, J=7.63, 0.75 Hz, 1H) 7.53 (dd, J=8.69, 1.56 Hz, 1H) 7.64 (t, J=7.94 Hz, 1H) 7.76 (m, J=8.63 Hz, 2H) 7.89-7.99 (m, 4H) 8.03 (d, J=8.63 Hz, 1H) 8.08 (d, J=1.13 Hz, 1H).

### Example 5: (4-(Aminomethyl)piperidin-1-yl)(5-(4-(trifluoromethyl)phenoxy)naphthalen-2-yl)methanone (Compound 5)

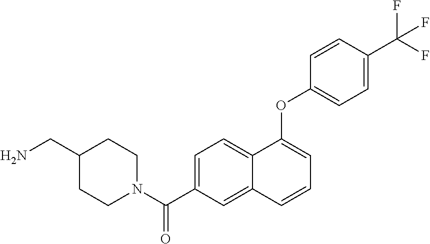 [SMILES: NCC1CCN(C(=O)c2ccc3c(Oc4ccc(C(F)(F)F)cc4)cccc3c2)CC1]

The title compound was synthesized following the procedure outlined for 3-aminoazetidin-1-yl)(5-(4-(trifluoromethyl)phenoxy)naphthalen-2-yl)methanone. LCMS: [M+H]⁺: 429.1. ¹H NMR: (400 MHz, DMSO-d6) δ ppm 1.19-1.31 (m, 2H) 1.67 (br s, 1H) 1.80-1.95 (m, 2H) 2.71-2.91 (m, 3H) 3.10 (br s, 1H) 4.54 (br s, 1H) 7.18 (d, J=8.50 Hz, 2H) 7.32 (d, J=7.00 Hz, 1H) 7.53 (dd, J=8.63, 1.50 Hz, 1H) 7.64 (t, J=7.94 Hz, 1H) 7.72-7.83 (m, 4H) 7.93 (d, J=8.38 Hz, 1H) 8.01 (d, J=8.63 Hz, 1H) 8.07 (d, J=0.88 Hz, 1H).

### Example 6: (3-Aminopiperidin-1-yl)(5-(4-(trifluoromethyl)phenoxy)naphthalen-2-yl)methanone (Compound 6)

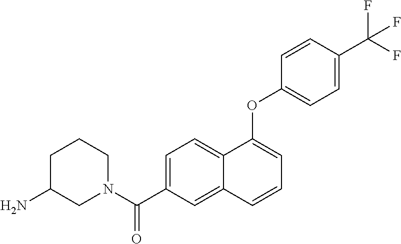 [SMILES: NC1CCCN(C(=O)c2ccc3c(Oc4ccc(C(F)(F)F)cc4)cccc3c2)C1]

The title compound was synthesized following the procedure outlined for 3-aminoazetidin-1-yl)(5-(4-(trifluoromethyl)phenoxy)naphthalen-2-yl)methanone. LCMS: [M+H]⁺: 415.0. ¹H NMR: (400 MHz, DMSO-d6) δ ppm 1.23-1.29 (m, 2H) 1.51-1.67 (m, 2H) 1.75 (br s, 1H) 2.02 (br s, 1H) 3.15 (br dd, J=7.28, 4.27 Hz, 2H) 7.18 (d, J=8.53 Hz, 2H) 7.33 (d, J=7.53 Hz, 1H) 7.59 (dd, J=8.66, 1.38 Hz, 1H) 7.65 (t, J=8.03 Hz, 1H) 7.76 (d, J=8.78 Hz, 2H) 7.94 (d, J=8.28 Hz, 2H) 8.03 (d, J=8.53 Hz, 2H) 8.13 (s, 1H).

### Example 7: (3-(Aminomethyl)azetidin-1-yl)(5-(4-(trifluoromethyl)phenoxy)naphthalen-2-yl)methanone (Compound 7)

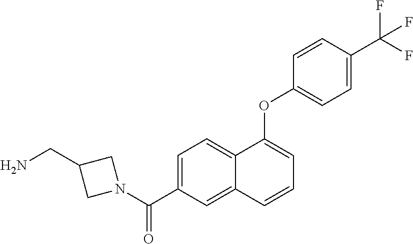 [SMILES: NCC1CN(C(=O)c2ccc3c(Oc4ccc(C(F)(F)F)cc4)cccc3c2)C1]

The title compound was synthesized following the procedure outlined for 3-aminoazetidin-1-yl)(5-(4-(trifluoromethyl)phenoxy)naphthalen-2-yl)methanone. LCMS: [M+H]⁺: 401.1. ¹H NMR: (400 MHz, DMSO-d6) δ ppm 2.86-2.98 (m, 1H) 3.15 (br t, J=5.65 Hz, 2H) 3.86-3.95 (m, 1H) 4.12-4.25 (m, 2H) 4.50 (br t, J=8.53 Hz, 1H) 7.17 (d, J=8.53 Hz, 2H) 7.35 (d, J=7.36 Hz, 1H) 7.65 (t, J=7.91 Hz, 1H) 7.73-7.79 (m, 3H) 7.87 (br s, 3H) 8.00 (t, J=8.41 Hz, 2H) 8.34 (d, J=1.25 Hz, 1H).

### Example 8: (3-(Aminomethyl)pyrrolidin-1-yl)(5-(4-(trifluoromethyl)phenoxy)naphthalen-2-yl)methanone (Compound 8)

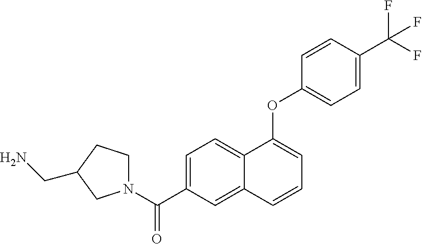 [SMILES: NCC1CCN(C(=O)c2ccc3c(Oc4ccc(C(F)(F)F)cc4)cccc3c2)C1]

The title compound was synthesized following the procedure outlined for 3-aminoazetidin-1-yl)(5-(4-(trifluoromethyl)phenoxy)naphthalen-2-yl)methanone. LCMS: [M+H]⁺: 415.1. ¹H NMR: (400 MHz, DMSO-d6) δ ppm 1.63-1.77 (m, 1H) 1.99-2.16 (m, 1H) 2.87 (dt, J=12.36, 6.24 Hz, 1H) 2.98 (br s, 1H) 3.26-3.37 (m, 1H) 3.45-3.60 (m, 5H) 3.60-3.67 (m, 3H) 3.72-3.80 (m, 1H) 7.17 (br d, J=7.03 Hz, 2H) 7.33 (t, J=6.53 Hz, 1H) 7.61-7.70 (m, 2H) 7.76 (br d, J=8.03 Hz, 3H) 7.85-8.03 (m, 3H) 8.24 (d, J=13.05 Hz, 1H).

### Example 9: (3-(Aminomethyl)piperidin-1-yl)(5-(4-(trifluoromethyl)phenoxy)naphthalen-2-yl)methanone (Compound 9)

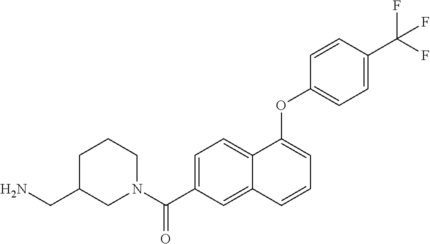 [SMILES: NCC1CCCN(C(=O)c2ccc3c(Oc4ccc(C(F)(F)F)cc4)cccc3c2)C1]

The title compound was synthesized following the procedure outlined for 3-aminoazetidin-1-yl)(5-(4-(trifluoromethyl)phenoxy)naphthalen-2-yl)methanone. LCMS: [M+H]⁺: 429.1. ¹H NMR: (400 MHz, DMSO-d6) δ ppm 1.30 (q, J=10.29 Hz, 1H) 1.39-1.53 (m, 1H) 1.62 (br s, 1H) 1.75-1.93 (m, 2H) 2.82 (br s, 2H) 3.08 (br s, 1H) 3.37-3.64 (m, 1H) 4.45 (br s, 1H) 7.18 (d, J=8.53 Hz, 2H) 7.31 (d, J=7.30 Hz, 1H) 7.54 (dd, J=8.53, 1.25 Hz, 1H) 7.63 (t, J=7.91 Hz, 1H) 7.75 (d, J=8.53 Hz, 2H) 7.96 (br d, J=8.28 Hz, 2H) 8.01 (d, J=8.53 Hz, 1H) 8.09 (br s, 1H).

### Example 10: N-((1-(5-(4-(trifluoromethyl)phenoxy)-2-naphthoyl)azetidin-3-yl)methyl)acetamide (Compound 10)

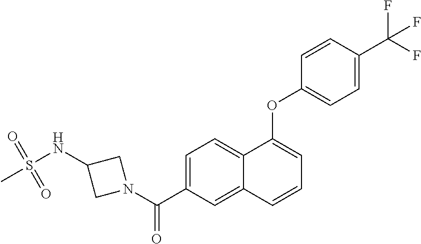 [SMILES: CS(=O)(=O)NC1CN(C(=O)c2ccc3c(Oc4ccc(C(F)(F)F)cc4)cccc3c2)C1]

The title compound was synthesized following the procedure outlined for tert-butyl (1-(5-(4-(trifluoromethyl)phenoxy)-2-naphthoyl)azetidin-3-yl)carbamate. LCMS: [M+H]⁺: 465.0. ¹H NMR: (400 MHz, DMSO-d6) δ ppm 2.93 (s, 3H) 3.96-4.07 (m, 1H) 4.21-4.33 (m, 2H) 4.38-4.47 (m, 1H) 4.71 (br s, 1H) 7.18 (d, J=8.50 Hz, 2H) 7.33-7.38 (m, 1H) 7.65 (t, J=7.94 Hz, 1H) 7.71-7.79 (m, 3H) 7.92 (br d, J=4.75 Hz, 1H) 8.02 (dd, J=8.57, 3.56 Hz, 2H) 8.33 (d, J=1.13 Hz, 1H).

### Example 11: (2,6-Diazaspiro[3.3]heptan-2-yl)(5-(4-(trifluoromethyl)phenoxy)naphthalen-2-yl)methanone (Compound 11)

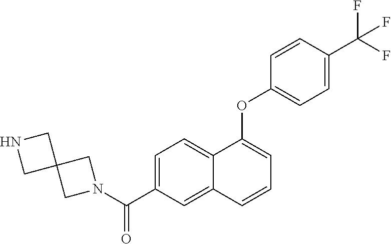 [SMILES: O=C(c1ccc2c(Oc3ccc(C(F)(F)F)cc3)cccc2c1)N1CC2(CNC2)C1]

The title compound was synthesized following the procedure outlined for 3-aminoazetidin-1-yl)(5-(4-(trifluoromethyl)phenoxy)naphthalen-2-yl)methanone. LCMS: [M+H]⁺: 413.2. ¹H NMR: (400 MHz, DMSO-d6) δ ppm 4.16 (dt, J=9.35, 5.99 Hz, 4H) 4.28 (s, 2H) 4.58 (s, 2H) 7.17 (d, J=8.28 Hz, 2H) 7.35 (d, J=7.77 Hz, 1H) 7.65 (t, J=7.91 Hz, 1H) 7.72-7.78 (m, 3H) 8.01 (t, J=8.16 Hz, 2H) 8.32 (d, J=1.25 Hz, 1H) 8.62 (br s, 2H).

### Example 12: 3-(Dimethylamino)azetidin-1-yl)(5-(4-(trifluoromethyl)phenoxy)naphthalen-2-yl)methanone (Compound 12)

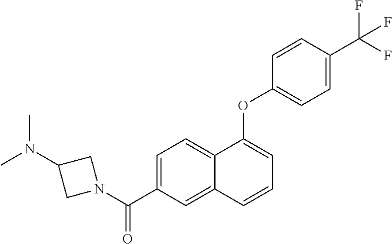 [SMILES: CN(C)C1CN(C(=O)c2ccc3c(Oc4ccc(C(F)(F)F)cc4)cccc3c2)C1]

The title compound was synthesized following the procedure outlined for tert-butyl (1-(5-(4-(trifluoromethyl)phenoxy)-2-naphthoyl)azetidin-3-yl)carbamate. LCMS: [M+H]⁺: 415.0. ¹H NMR: (400 MHz, DMSO-d6) δ ppm 2.12 (br s, 6H) 3.14 (br s, 1H) 3.89 (br dd, J=9.13, 4.38 Hz, 1H) 4.06-4.16 (m, 1H) 4.17-4.24 (m, 1H) 4.33-4.46 (m, 1H) 7.17 (d, J=8.50 Hz, 2H) 7.31-7.39 (m, 1H) 7.64 (t, J=7.94 Hz, 1H) 7.73-7.81 (m, 3H) 7.94-8.06 (m, 3H) 8.36 (d, J=1.25 Hz, 1H).

### Example 13: (3-(Hydroxymethyl)azetidin-1-yl)(5-(4-(trifluoromethyl)phenoxy)naphthalen-2-yl)methanone (Compound 13)

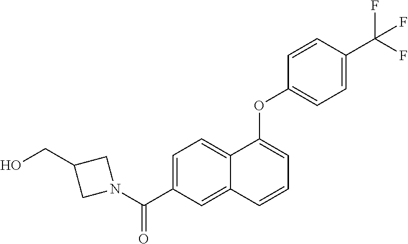 [SMILES: O=C(c1ccc2c(Oc3ccc(C(F)(F)F)cc3)cccc2c1)N1CC(CO)C1]

The title compound was synthesized following the procedure outlined for tert-butyl (1-(5-(4-(trifluoromethyl)phenoxy)-2-naphthoyl)azetidin-3-yl)carbamate. LCMS: [M+H]⁺: 402.1. ¹H NMR: (400 MHz, DMSO-d6) δ ppm 3.57 (t, J=5.75 Hz, 2H) 3.84 (dd, J=9.69, 5.44 Hz, 1H) 4.05-4.15 (m, 2H) 4.41 (t, J=8.44 Hz, 1H) 4.84 (t, J=5.32 Hz, 1H) 7.17 (d, J=8.50 Hz, 2H) 7.34 (dd, J=7.50, 0.75 Hz, 1H) 7.64 (t, J=7.94 Hz, 1H) 7.70-7.80 (m, 3H) 7.96 (s, 1H) 8.00 (dd, J=8.50, 4.88 Hz, 2H) 8.34 (d, J=1.25 Hz, 1H).

### Example 14: (3-((Dimethylamino)methyl)azetidin-1-yl)(5-(4-(trifluoromethyl)phenoxy)naphthalen-2-yl)methanone (Compound 14)

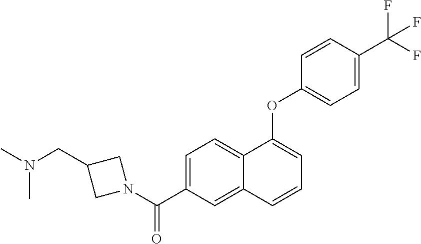 [SMILES: CN(C)CC1CN(C(=O)c2ccc3c(Oc4ccc(C(F)(F)F)cc4)cccc3c2)C1]

The title compound was synthesized following the procedure outlined for tert-butyl (1-(5-(4-(trifluoromethyl)phenoxy)-2-naphthoyl)azetidin-3-yl)carbamate. LCMS: [M+H]⁺: 429.1. ¹H NMR: (400 MHz, DMSO-d6) δ ppm 2.18 (s, 6H) 2.57 (br d, J=7.03 Hz, 2H) 3.74 (br dd, J=9.79, 5.52 Hz, 1H) 4.05 (br dd, J=8.28, 6.02 Hz, 1H) 4.11-4.25 (m, 1H) 4.47 (br t, J=8.41 Hz, 1H) 7.17 (d, J=8.53 Hz, 2H) 7.34 (d, J=7.03 Hz, 1H) 7.64 (t, J=8.03 Hz, 1H) 7.72-7.79 (m, 3H) 7.93-8.05 (m, 3H) 8.34 (s, 1H).

### Example 15: (3-Hydroxyazetidin-1-yl)(5-(4-(trifluoromethyl)phenoxy)naphthalen-2-yl)methanone (Compound 15)

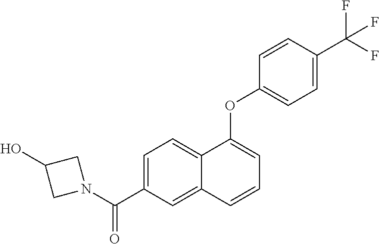 [SMILES: O=C(c1ccc2c(Oc3ccc(C(F)(F)F)cc3)cccc2c1)N1CC(O)C1]

The title compound was synthesized following the procedure outlined for tert-butyl (1-(5-(4-(trifluoromethyl)phenoxy)-2-naphthoyl)azetidin-3-yl)carbamate. LCMS: [M+H]⁺: 388.0.

### Example 16: N-((1-(5-(4-(trifluoromethyl)phenoxy)-2-naphthoyl)azetidin-3-yl)methyl)acetamide (Compound 16)

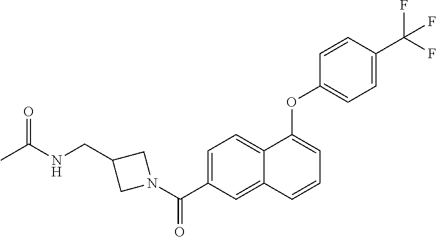 [SMILES: CC(=O)NCC1CN(C(=O)c2ccc3c(Oc4ccc(C(F)(F)F)cc4)cccc3c2)C1]

The title compound was synthesized following the procedure outlined for N-(1-(5-(4-(trifluoromethyl)phenoxy)-2-naphthoyl)azetidin-3-yl)acetamide. LCMS: [M+H]⁺: 443.1. ¹H NMR: (400 MHz, DMSO-d6) δ ppm 1.81 (s, 3H) 2.71-2.81 (m, 1H) 3.30 (t, J=6.38 Hz, 2H) 4.04 (br dd, J=8.57, 5.44 Hz, 1H) 4.11 (br t, J=9.19 Hz, 1H) 4.44 (br t, J=8.50 Hz, 1H) 7.17 (d, J=8.50 Hz, 2H) 7.34 (d, J=7.50 Hz, 1H) 7.64 (t, J=7.88 Hz, 1H) 7.75 (d, J=8.63 Hz, 3H) 7.97-8.07 (m, 3H) 8.33 (d, J=1.38 Hz, 1H).

### Example 17: N-((1-(5-(4-(trifluoromethyl)phenoxy)-2-naphthoyl)azetidin-3-yl)methyl)methanesulfonamide (Compound 17)

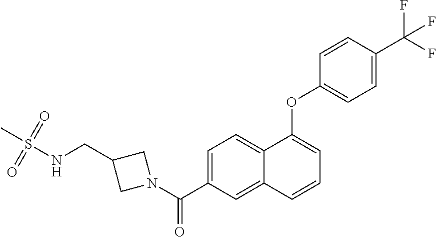 [SMILES: CS(=O)(=O)NCC1CN(C(=O)c2ccc3c(Oc4ccc(C(F)(F)F)cc4)cccc3c2)C1]

The title compound was synthesized following the procedure outlined for N-(1-(5-(4-(trifluoromethyl)phenoxy)-2-naphthoyl)azetidin-3-yl)acetamide. LCMS: [M+H]⁺: 479.1. ¹H NMR: (400 MHz, DMSO-d6) δ ppm 2.82 (dt, J=13.55, 6.78 Hz, 1H) 2.92 (s, 3H) 3.16-3.25 (m, 2H) 3.84 (br dd, J=10.04, 5.52 Hz, 1H) 4.02-4.20 (m, 2H) 4.46 (br t, J=8.53 Hz, 1H) 7.15-7.25 (m, 3H) 7.34 (d, J=7.03 Hz, 1H) 7.64 (t, J=7.91 Hz, 1H) 7.73-7.78 (m, 3H) 7.97-8.03 (m, 2H) 8.33 (s, 1H).

### Example 18: (6-Methyl-2,6-diazaspiro[3.3]heptan-2-yl)(5-(4-(trifluoromethyl)phenoxy)naphthalen-2-yl)methanone (Compound 18)

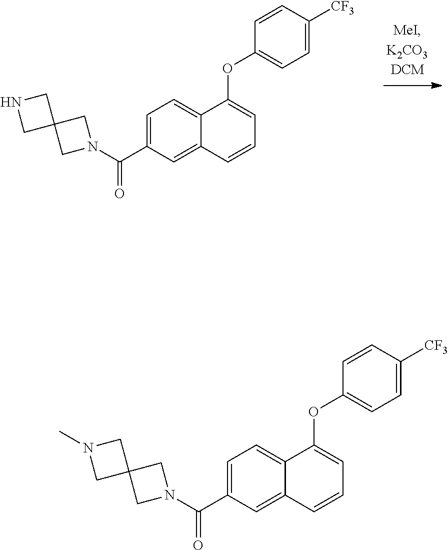 [SMILES: O=C(c1ccc2c(Oc3ccc(C(F)(F)F)cc3)cccc2c1)N1CC2(CNC2)C1 ; CN1CC2(C1)CN(C(=O)c1ccc3c(Oc4ccc(C(F)(F)F)cc4)cccc3c1)C2]

(2,6-Diazaspiro[3.3]heptan-2-yl)(5-(4-(trifluoromethyl)phenoxy)naphthalen-2-yl)methanone (10 mg, 1 eq.), synthesized following the procedure outlined for tert-butyl (1-(5-(4-(trifluoromethyl)phenoxy)-2-naphthoyl)azetidin-3-yl)carbamate), K₂CO₃ (10 mg, 3 eq.), and DMF (0.3 mL) were stirred at 0° C. Mel (3 μL, 1.5 eq.) was added carefully and warmed to 23° C. and stirred 2 hr. Upon completion, the reaction mixture was acidified with conc. HCl, and the mixture was purified by semi-prep HPLC (Luna C18, 5 μm, 100 Å, 250×10 mm, ACN+0.1% TFA:H₂O+0.1% TFA, gradient) to give the desired product as the TFA salt (2.8 mg, 22% yield). LCMS: [M+H]⁺: 427.0.

### Example 19: (6-Ethyl-2,6-diazaspiro[3.3]heptan-2-yl)(5-(4-(trifluoromethyl)phenoxy)naphthalen-2-yl)methanone (Compound 19)

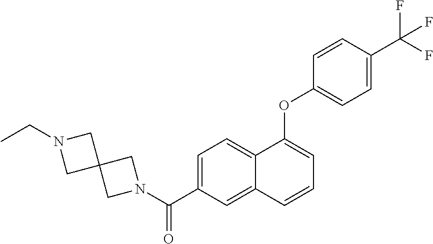 [SMILES: CCN1CC2(C1)CN(C(=O)c1ccc3c(Oc4ccc(C(F)(F)F)cc4)cccc3c1)C2]

The title compound was synthesized following the procedure outlined for (6-methyl-2,6-diazaspiro[3.3]heptan-2-yl)(5-(4-(trifluoromethyl)phenoxy)naphthalen-2-yl)methanone. LCMS: [M+H]⁺: 441.1. ¹H NMR: (400 MHz, DMSO-d6) δ ppm 0.86 (t, J=7.03 Hz, 3H) 1.95-2.05 (m, 1H) 2.40 (br dd, J=2.64, 1.63 Hz, 1H) 4.18 (s, 2H) 4.48 (s, 2H) 7.17 (d, J=8.53 Hz, 2H) 7.34 (d, J=7.53 Hz, 1H) 7.60-7.66 (m, 1H) 7.71-7.80 (m, 3H) 8.00 (dd, J=8.41, 3.39 Hz, 2H) 8.34 (s, 1H).

### Example 20: (6-Isopropyl-2,6-diazaspiro[3.3]heptan-2-yl)(5-(4-(trifluoromethyl)phenoxy)naphthalen-2-yl)methanone (Compound 20)

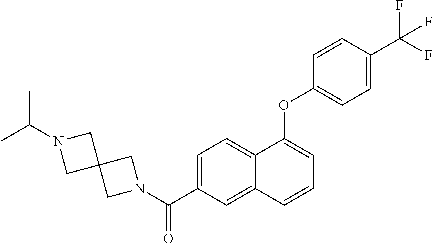 [SMILES: CC(C)N1CC2(CN(C(=O)c3ccc4c(Oc5ccc(C(F)(F)F)cc5)cccc4c3)C2)C1]

The title compound was synthesized following the procedure outlined for (6-methyl-2,6-diazaspiro[3.3]heptan-2-yl)(5-(4-(trifluoromethyl)phenoxy)naphthalen-2-yl)methanone. LCMS: [M+H]⁺: 455.1. ¹H NMR: (400 MHz, DMSO-d6) δ ppm 1.11 (br d, J=3.76 Hz, 6H) 1.91-2.06 (m, 1H) 4.17-4.33 (m, 5H) 4.36 (s, 1H) 4.52 (s, 1H) 4.65 (s, 1H) 7.17 (d, J=8.53 Hz, 2H) 7.36 (d, J=7.53 Hz, 1H) 7.66 (t, J=7.91 Hz, 1H) 7.76 (br d, J=8.53 Hz, 3H) 7.93-8.05 (m, 2H) 8.33 (br d, J=4.27 Hz, 1H) 9.71-9.96 (m, 1H).

### Example 21: (6-(2-Hydroxyethyl)-2,6-diazaspiro[3.3]heptan-2-yl)(5-(4-(trifluoromethyl)phenoxy)naphthalen-2-yl)methanone (Compound 21)

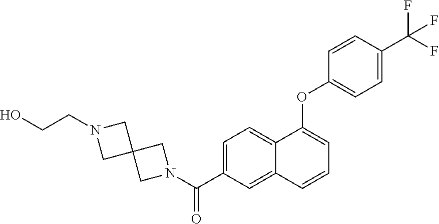 [SMILES: O=C(c1ccc2c(Oc3ccc(C(F)(F)F)cc3)cccc2c1)N1CC2(CN(CCO)C2)C1]

The title compound was synthesized following the procedure outlined for (6-methyl-2,6-diazaspiro[3.3]heptan-2-yl)(5-(4-(trifluoromethyl)phenoxy)naphthalen-2-yl)methanone. LCMS: [M+H]⁺: 457.0.

### Example 22: (6-(2-Methoxyethyl)-2,6-diazaspiro[3.3]heptan-2-yl)(5-(4-(trifluoromethyl)phenoxy)naphthalen-2-yl)methanone (Compound 22)

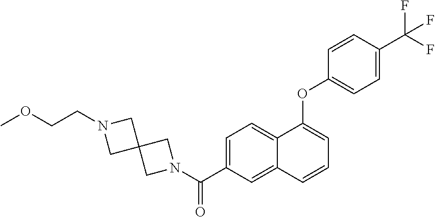 [SMILES: COCCN1CC2(C1)CN(C(=O)c1ccc3c(Oc4ccc(C(F)(F)F)cc4)cccc3c1)C2]

The title compound was synthesized following the procedure outlined for (6-methyl-2,6-diazaspiro[3.3]heptan-2-yl)(5-(4-(trifluoromethyl)phenoxy)naphthalen-2-yl)methanone. LCMS: [M+H]⁺: 471.1. ¹H NMR: (400 MHz, DMSO-d6) δ ppm 3.29 (s, 3H) 3.49 (br s, 3H) 4.16-4.38 (m, 6H) 4.51 (s, 1H) 4.62 (s, 1H) 7.17 (br d, J=8.53 Hz, 2H) 7.36 (d, J=7.53 Hz, 1H) 7.66 (t, J=7.91 Hz, 1H) 7.76 (br d, J=8.53 Hz, 3H) 8.01 (br t, J=8.16 Hz, 2H) 8.32 (br d, J=3.51 Hz, 1H) 9.71-9.86 (m, 1H).

### Example 23: (6-(2-Fluoroethyl)-2,6-diazaspiro[3.3]heptan-2-yl)(5-(4-(trifluoromethyl)phenoxy)naphthalen-2-yl)methanone (Compound 23)

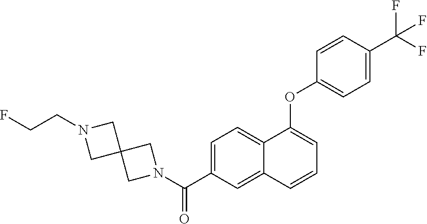 [SMILES: O=C(c1ccc2c(Oc3ccc(C(F)(F)F)cc3)cccc2c1)N1CC2(CN(CCF)C2)C1]

The title compound was synthesized following the procedure outlined for (6-methyl-2,6-diazaspiro[3.3]heptan-2-yl)(5-(4-(trifluoromethyl)phenoxy)naphthalen-2-yl)methanone. LCMS: [M+H]⁺: 459.1.

### Example 24: (6-(2,2-Difluoroethyl)-2,6-diazaspiro[3.3]heptan-2-yl)(5-(4-(trifluoromethyl)phenoxy)naphthalen-2-yl)methanone (Compound 24)

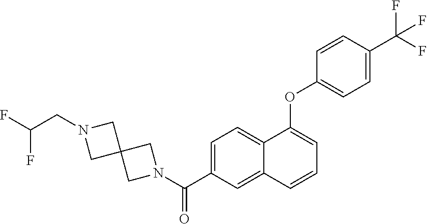 [SMILES: O=C(c1ccc2c(Oc3ccc(C(F)(F)F)cc3)cccc2c1)N1CC2(CN(CC(F)F)C2)C1]

The title compound was synthesized following the procedure outlined for (6-methyl-2,6-diazaspiro[3.3]heptan-2-yl)(5-(4-(trifluoromethyl)phenoxy)naphthalen-2-yl)methanone. LCMS: [M+H]⁺: 477.1.

### Example 25: (3-((Methylamino)methyl)azetidin-1-yl)(5-(4-(trifluoromethyl)phenoxy)naphthalen-2-yl)methanone (Compound 25)

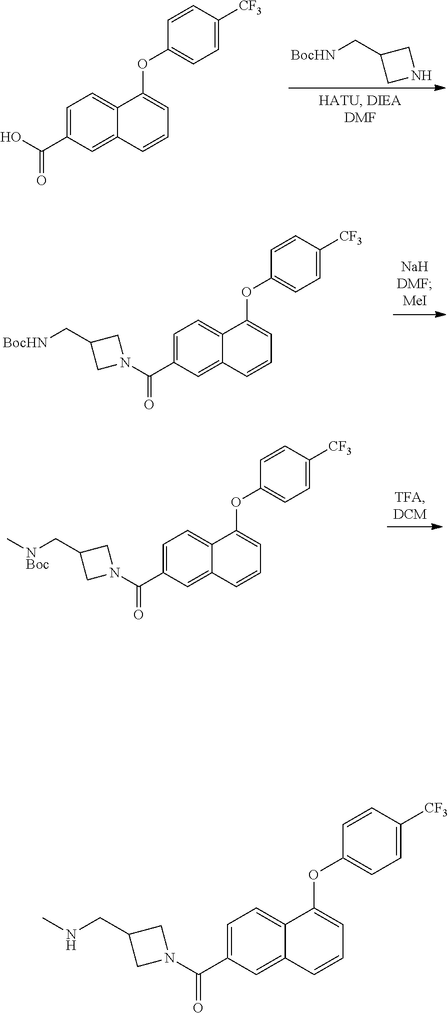 [SMILES: O=C(O)c1ccc2c(Oc3ccc(C(F)(F)F)cc3)cccc2c1 ; CC(C)(C)OC(=O)NCC1CNC1 ; CC(C)(C)OC(=O)NCC1CN(C(=O)c2ccc3c(Oc4ccc(C(F)(F)F)cc4)cccc3c2)C1 ; CN(CC1CN(C(=O)c2ccc3c(Oc4ccc(C(F)(F)F)cc4)cccc3c2)C1)C(=O)OC(C)(C)C ; CNCC1CN(C(=O)c2ccc3c(Oc4ccc(C(F)(F)F)cc4)cccc3c2)C1]

tert-Butyl ((1-(5-(4-(trifluoromethyl)phenoxy)-2-naphthoyl)azetidin-3-yl)methyl)carbamate: 5-(4-(Trifluoromethyl)phenoxy)-2-naphthoic acid (66 mg, 0.2 mmol, 1 eq.), tert-butyl (azetidin-3-ylmethyl)carbamate HCl (49 mg, 0.22 mmol, 1.1 eq.), HATU (91 mg, 0.24 mmol, 1.2 eq.), DIEA (87 μL, 0.5 mmol, 2.5 eq.), and DMF (1 mL, 0.2 M) were stirred at 23° C. until LCMS indicated complete conversion of the starting material, 2 hr. The reaction mixture was diluted with H₂O, and the resulting precipitate was filtered, rinsed with H₂O, and dried to give tert-butyl ((1-(5-(4-(trifluoromethyl)phenoxy)-2-naphthoyl)azetidin-3-yl)methyl)carbamate (68 mg, 68% yield).

tert-Butyl methyl((1-(5-(4-(trifluoromethyl)phenoxy)-2-naphthoyl)azetidin-3-yl)methyl)carbamate: tert-Butyl ((1-(5-(4-(trifluoromethyl)phenoxy)-2-naphthoyl)azetidin-3-yl)methyl)carbamate (12 mg, 1 eq.) was dissolved in DMF (1 mL) and cooled to 0° C. NaH (2 mg, 2 eq., 60% in mineral oil) was carefully added and the mixture was stirred 30 min. Mel (3 μL, 1.5 eq.) was carefully added and the mixture was stirred 2 hr. Upon completion, the reaction was quenched with sat. aq. NH₄Cl, diluted with EtOAc, and the organic layer was washed with H₂O, brine, and dried on Na₂SO₄. The residue was used directly in the next step without further purification.

(3-((Methylamino)methyl)azetidin-1-yl)(5-(4-(trifluoromethyl)phenoxy)-naphthalen-2-yl)methanone: tert-Butyl methyl((1-(5-(4-(trifluoromethyl)phenoxy)-2-naphthoyl)azetidin-3-yl)methyl)carbamate (crude from the previous step, 1 eq.) was dissolved in DCM (0.8 mL), and TFA (0.2 mL) was added carefully. The reaction mixture was stirred at 23° C. until LCMS indicated complete conversion to the desired product, 1 hr. Upon completion, mixture was concentrated, and the residue was purified by semi-prep HPLC (Luna C18, 5 μm, 100 Å, 250×10 mm, ACN+0.1% TFA:H₂O+0.1% TFA, gradient) to give the desired product as the TFA salt (5 mg, 40% yield over 2 steps). LCMS: [M+H]⁺: 415.1. ¹H NMR: (400 MHz, DMSO-d6) δ ppm 2.57 (br s, 2H) 2.91-3.13 (m, 1H) 3.15-3.28 (m, 2H) 3.93 (br dd, J=10.04, 5.52 Hz, 1H) 4.10-4.28 (m, 2H) 4.53 (br t, J=8.53 Hz, 1H) 7.17 (d, J=8.53 Hz, 2H) 7.33-7.38 (m, 1H) 7.65 (t, J=7.91 Hz, 1H) 7.76 (d, J=9.03 Hz, 2H) 8.00 (t, J=8.03 Hz, 2H) 8.29-8.38 (m, 1H) 8.43 (br s, 1H).

### Example 26: (3-(Methylamino)azetidin-1-yl)(5-(4-(trifluoromethyl)phenoxy)naphthalen-2-yl)methanone (Compound 26)

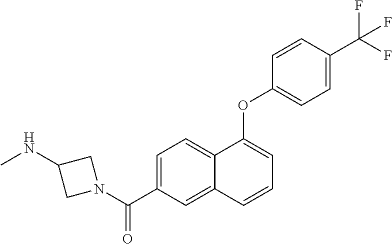 [SMILES: CNC1CN(C(=O)c2ccc3c(Oc4ccc(C(F)(F)F)cc4)cccc3c2)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 401.1. ¹H NMR: (400 MHz, DMSO-d6) δ ppm 2.56-2.63 (m, 3H) 4.09 (br s, 1H) 4.17 (br d, J=11.80 Hz, 1H) 4.25-4.49 (m, 2H) 4.71 (br t, J=7.91 Hz, 1H) 6.55 (br s, 1H) 7.18 (d, J=8.53 Hz, 2H) 7.37 (d, J=7.03 Hz, 1H) 7.67 (t, J=7.91 Hz, 1H) 7.73-7.80 (m, 3H) 8.00-8.07 (m, 2H) 8.35 (s, 1H) 9.10 (brs, 2H).

### Example 27: (3-(Ethylamino)azetidin-1-yl)(5-(4-(trifluoromethyl)phenoxy)naphthalen-2-yl)methanone (Compound 27)

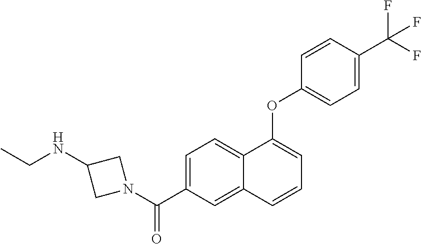 [SMILES: CCNC1CN(C(=O)c2ccc3c(Oc4ccc(C(F)(F)F)cc4)cccc3c2)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 415.1. ¹H NMR: (400 MHz, DMSO-d6) δ ppm 1.18 (t, J=7.28 Hz, 3H) 2.97 (br s, 2H) 4.19 (br d, J=10.79 Hz, 2H) 4.27-4.48 (m, 2H) 4.73 (br t, J=8.28 Hz, 1H) 7.18 (d, J=8.53 Hz, 2H) 7.37 (d, J=7.53 Hz, 1H) 7.67 (t, J=8.03 Hz, 1H) 7.73-7.80 (m, 3H) 8.04 (dd, J=8.41, 3.89 Hz, 2H) 8.33-8.36 (m, 1H) 9.20 (br s, 2H).

### Example 28: (3-(Isopropylamino)azetidin-1-yl)(5-(4-(trifluoromethyl)phenoxy)naphthalen-2-yl)methanone (Compound 28)

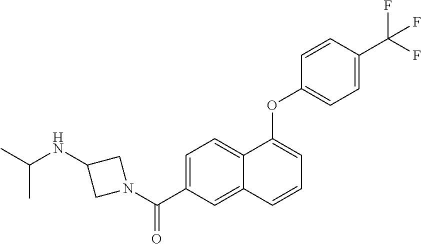 [SMILES: CC(C)NC1CN(C(=O)c2ccc3c(Oc4ccc(C(F)(F)F)cc4)cccc3c2)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 429.1.

### Example 29: (3-((2-Methoxyethyl)amino)azetidin-1-yl)(5-(4-(trifluoromethyl)phenoxy)naphthalen-2-yl)methanone (Compound 29)

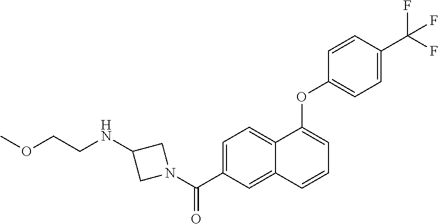 [SMILES: COCCNC1CN(C(=O)c2ccc3c(Oc4ccc(C(F)(F)F)cc4)cccc3c2)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 445.0. ¹H NMR: (400 MHz, DMSO-d6) δ ppm 2.63 (br t, J=5.27 Hz, 3H) 3.24 (s, 3H) 3.63 (br s, 1H) 3.78 (br dd, J=10.04, 5.27 Hz, 1H) 4.04 (br dd, J=8.41, 5.40 Hz, 1H) 4.15-4.26 (m, 1H) 4.48 (br t, J=7.91 Hz, 1H) 7.17 (d, J=8.53 Hz, 2H) 7.34 (d, J=7.53 Hz, 1H) 7.63 (t, J=8.03 Hz, 1H) 7.75 (br d, J=8.78 Hz, 3H) 8.00 (dd, J=8.53, 4.27 Hz, 2H) 8.33 (s, 1H).

### Example 30: (3-((2-Hydroxyethyl)amino)azetidin-1-yl)(5-(4-(trifluoromethyl)phenoxy)naphthalen-2-yl)methanone (Compound 30)

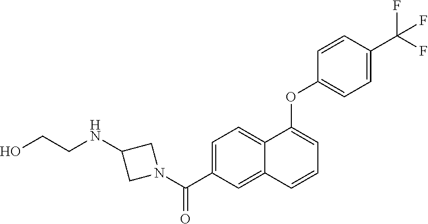 [SMILES: O=C(c1ccc2c(Oc3ccc(C(F)(F)F)cc3)cccc2c1)N1CC(NCCO)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 431.1. ¹H NMR: (400 MHz, DMSO-d6) δ ppm 3.03 (br d, J=3.13 Hz, 1H) 3.63 (br d, J=4.25 Hz, 1H) 4.01-4.45 (m, 4H) 4.73 (br s, 1H) 5.27-5.43 (m, 1H) 6.56 (br s, 1H) 7.18 (d, J=8.63 Hz, 2H) 7.37 (d, J=7.63 Hz, 1H) 7.63-7.70 (m, 1H) 7.76 (br d, J=8.75 Hz, 3H) 7.99-8.08 (m, 2H) 8.34 (s, 2H) 9.22 (br dd, J=7.44, 2.31 Hz, 1H).

### Example 31: (3-((2-Fluoroethyl)amino)azetidin-1-yl)(5-(4-(trifluoromethyl)phenoxy)naphthalen-2-yl)methanone (Compound 31)

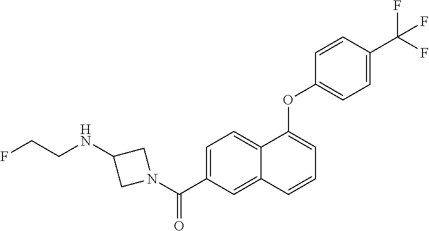 [SMILES: O=C(c1ccc2c(Oc3ccc(C(F)(F)F)cc3)cccc2c1)N1CC(NCCF)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 433.0.

### Example 32: (3-((2,2-Difluoroethyl)amino)azetidin-1-yl)(5-(4-(trifluoromethyl)phenoxy)naphthalen-2-yl)methanone (Compound 32)

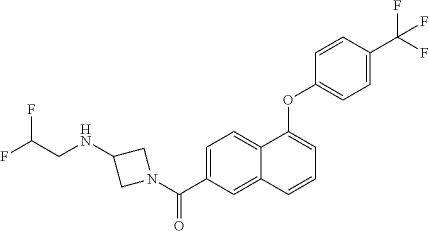 [SMILES: O=C(c1ccc2c(Oc3ccc(C(F)(F)F)cc3)cccc2c1)N1CC(NCC(F)F)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 451.0.

### Example 33: (3-((Ethylamino)methyl)azetidin-1-yl)(5-(4-(trifluoromethyl)-phenoxy)naphthalen-2-yl)methanone (Compound 33)

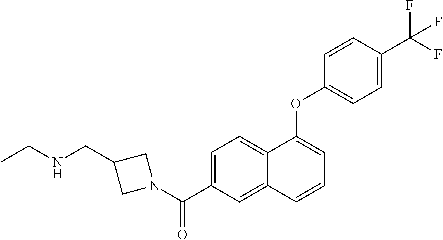 [SMILES: CCNCC1CN(C(=O)c2ccc3c(Oc4ccc(C(F)(F)F)cc4)cccc3c2)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 429.1. ¹H NMR: (400 MHz, DMSO-d6) δ ppm 1.12-1.25 (m, 4H) 2.91-3.02 (m, 3H) 3.14-3.30 (m, 3H) 3.92 (br dd, J=10.04, 5.52 Hz, 1H) 4.11-4.28 (m, 2H) 4.54 (br t, J=8.53 Hz, 1H) 7.17 (d, J=8.53 Hz, 2H) 7.36 (d, J=7.03 Hz, 1H) 7.65 (t, J=7.91 Hz, 1H) 7.76 (d, J=8.78 Hz, 3H) 8.00 (t, J=8.03 Hz, 2H) 8.26-8.43 (m, 3H).

### Example 34: (3-((Isopropylamino)methyl)azetidin-1-yl)(5-(4-(trifluoromethyl)phenoxy)-naphthalen-2-yl)methanone (Compound 34)

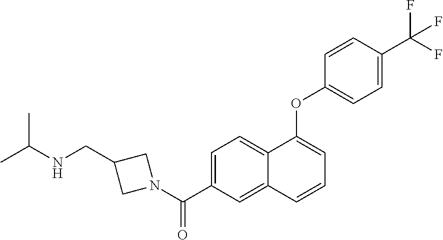 [SMILES: CC(C)NCC1CN(C(=O)c2ccc3c(Oc4ccc(C(F)(F)F)cc4)cccc3c2)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 443.1.

### Example 35: (3-(((2-Methoxyethyl)amino)methyl)azetidin-1-yl)(5-(4-(trifluoromethyl)-phenoxy)naphthalen-2-yl)methanone (Compound 35)

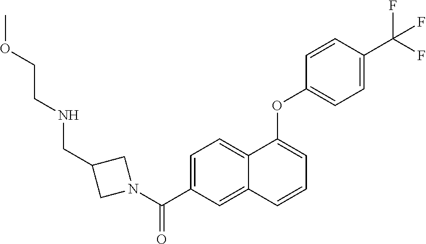 [SMILES: COCCNCC1CN(C(=O)c2ccc3c(Oc4ccc(C(F)(F)F)cc4)cccc3c2)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 459.1.

### Example 36: (3-(((2-Fluoroethyl)amino)methyl)azetidin-1-yl)(5-(4-(trifluoromethyl)-phenoxy)naphthalen-2-yl)methanone (Compound 36)

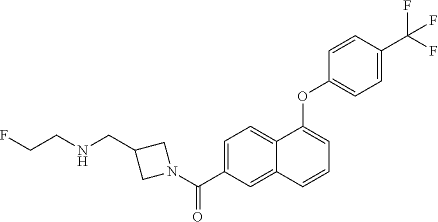 [SMILES: O=C(c1ccc2c(Oc3ccc(C(F)(F)F)cc3)cccc2c1)N1CC(CNCCF)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 447.1. ¹H NMR: (400 MHz, DMSO-d6) δ ppm 2.95-3.08 (m, 1H) 3.93 (br dd, J=9.91, 5.65 Hz, 1H) 4.13-4.25 (m, 2H) 4.54 (br t, J=8.28 Hz, 1H) 4.62-4.68 (m, 1H) 4.73-4.82 (m, 1H) 7.17 (d, J=8.53 Hz, 2H) 7.36 (d, J=7.03 Hz, 1H) 7.65 (t, J=8.03 Hz, 1H) 7.76 (d, J=8.78 Hz, 3H) 8.00 (t, J=7.91 Hz, 2H) 8.34 (d, J=1.00 Hz, 1H).

### Example 37: (3-(((2-Hydroxyethyl)amino)methyl)azetidin-1-yl)(5-(4-(trifluoromethyl)-phenoxy)naphthalen-2-yl)methanone (Compound 37)

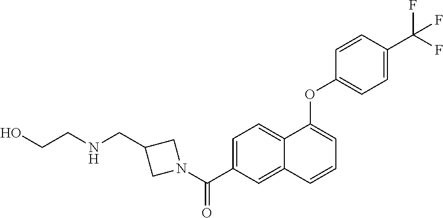 [SMILES: O=C(c1ccc2c(Oc3ccc(C(F)(F)F)cc3)cccc2c1)N1CC(CNCCO)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 445.0.

### Example 38: (3-(((2,2-Fifluoroethyl)amino)methyl)azetidin-1-yl)(5-(4-(trifluoromethyl)-phenoxy)naphthalen-2-yl)methanone (Compound 38)

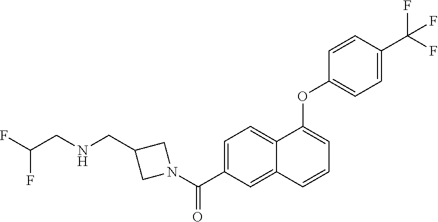 [SMILES: O=C(c1ccc2c(Oc3ccc(C(F)(F)F)cc3)cccc2c1)N1CC(CNCC(F)F)C1]

The title compound was synthesized following the procedure outlined for (3-((methylamino)methyl)azetidin-1-yl)(5-(4-(trifluoromethyl)phenoxy)naphthalen-2-yl)methanone. LCMS: [M+H]⁺: 465.1.

### Example 39: (3-(Pyridin-2-ylamino)azetidin-1-yl)(5-(4-(trifluoromethyl)-phenoxy)naphthalen-2-yl)methanone (Compound 39)

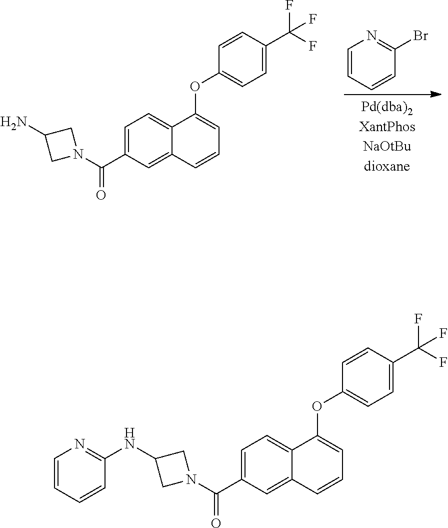

(3-Aminoazetidin-1-yl)(5-(4-(trifluoromethyl)phenoxy)naphthalen-2-yl)methanone TFA salt (10 mg, 1 eq.), 2-bromopyridine (4 mg, 1.3 eq.), XantPhos (1 mg, 0.05 eq.), NaOtBu (6 mg, 3 eq.), and dioxane (1 mL) were thoroughly purged with N₂ for 20 min. To this mixture was added Pd(dba)₂ (1 mg, 0.05 eq.) and the reaction was heated to 90° C. for 4 hr. Upon completion, the reaction mixture was diluted with diluted with EtOAc, and the organic layer was washed with sat. aq. NH₄Cl, H₂O, brine, and dried on Na₂SO₄. The residue was purified by semi-prep HPLC (Luna C18, 5 μm, 100 Å, 250×10 mm, ACN+0.1% TFA:H₂O+0.1% TFA, gradient) to give the desired product (1 mg, 11% yield). LCMS: [M+H]⁺: 464.1.

### Example 40: (3-((Pyridin-2-ylamino)methyl)azetidin-1-yl)(5-(4-(trifluoromethyl)-phenoxy)naphthalen-2-yl)methanone (Compound 40)

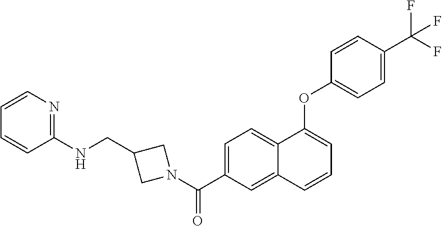 [SMILES: O=C(c1ccc2c(Oc3ccc(C(F)(F)F)cc3)cccc2c1)N1CC(CNc2ccccn2)C1]

The title compound was synthesized following the procedure outlined for ((3-(pyridin-2-ylamino)azetidin-1-yl)(5-(4-(trifluoromethyl)phenoxy)naphthalen-2-yl)methanone. LCMS: [M+H]⁺: 478.0. ¹H NMR: (400 MHz, DMSO-d6) δ ppm 2.89-3.03 (m, 1H) 3.56-3.64 (m, 2H) 3.85 (br dd, J=10.04, 5.27 Hz, 1H) 4.08-4.26 (m, 2H) 4.52 (br t, J=8.78 Hz, 1H) 7.17 (d, J=8.53 Hz, 2H) 7.35 (d, J=7.03 Hz, 1H) 7.65 (t, J=7.91 Hz, 1H) 7.72-7.82 (m, 4H) 7.94 (d, J=5.02 Hz, 1H) 8.00 (dd, J=8.53, 3.51 Hz, 2H) 8.35 (s, 1H).

### Example 41: (6-(Pyridin-2-yl)-2,6-diazaspiro[3.3]heptan-2-yl)(5-(4-(trifluoromethyl)-phenoxy)naphthalen-2-yl)methanone (Compound 41)

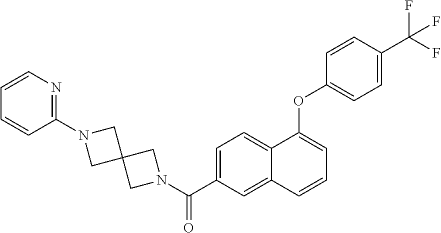 [SMILES: O=C(c1ccc2c(Oc3ccc(C(F)(F)F)cc3)cccc2c1)N1CC2(C1)CN(c1ccccn1)C2]

The title compound was synthesized following the procedure outlined for ((3-(pyridin-2-ylamino)azetidin-1-yl)(5-(4-(trifluoromethyl)phenoxy)naphthalen-2-yl)methanone. LCMS: [M+H]⁺: 490.0.

### Example 42: N,N-dimethyl-5-(4-(trifluoromethyl)phenoxy)-2-naphthamide (Compound 42)

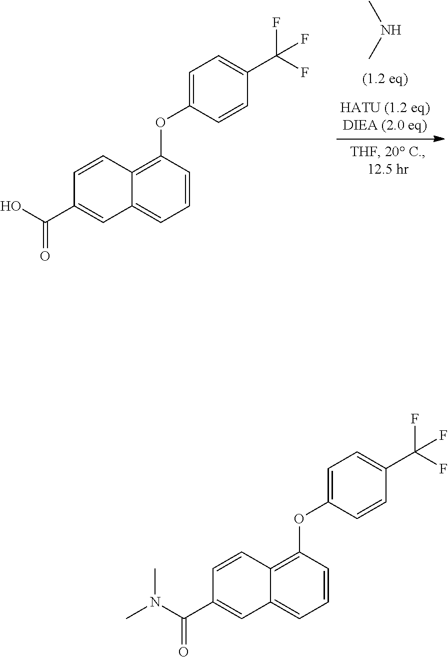

To a solution of 5-(4-(Trifluoromethyl)phenoxy)-2-naphthoic acid (50 mg, 0.15 mmol, 1 eq) and HATU (58.16 mg, 0.18 mmol, 1.2 eq) in DCM (1 mL) was added DIEA (38.90 mg, 0.30 mmol, 52.4 uL, 2 eq). The mixture was stirred at 20° C. for 0.5 h. N-methylmethanamine (2 M, 90.3 uL, 1.2 eq) in THF was added into the mixture. The resulting mixture was stirred at 20° C. for 12 h. LCMS detected the desired compound. The mixture was diluted with water (10 mL), extracted with EtOAc (20 mL*3). The combined organic layer was washed with brine (20 mL), dried over Na₂SO₄, filtered and concentrated. The residue was purified by prep-HPLC (column: Waters Xbridge 150*50 10 u; mobile phase: [water (0.05% ammonia hydroxide v/v)-ACN]; B %: 56%-86%, 7.8 min) to give the title compound (13.8 mg, 38.6 umol, 25.6% yield) as white solid. LCMS (ESI): mass calcd. for C₂₀H₁₆F₃NO₂ 359.11, m/z found 360.0 [M+H]⁺; ¹H NMR (400 MHz, CDCl₃) δ 8.09 (d, J=8.8 Hz, 1H), 7.97 (s, 1H), 7.73 (d, J=8.4 Hz, 1H), 7.58 (d, J=8.8 Hz, 2H), 7.53-7.49 (m, 2H), 7.12 (d, J=7.2 Hz, 1H), 7.06 (d, J=8.8 Hz, 2H), 3.17 (s, 3H), 3.04 (s, 3H).

### Example 43: N-cyano-N-methyl-5-(4-(trifluoromethyl)phenoxy)-2-naphthamide (Compound 43)

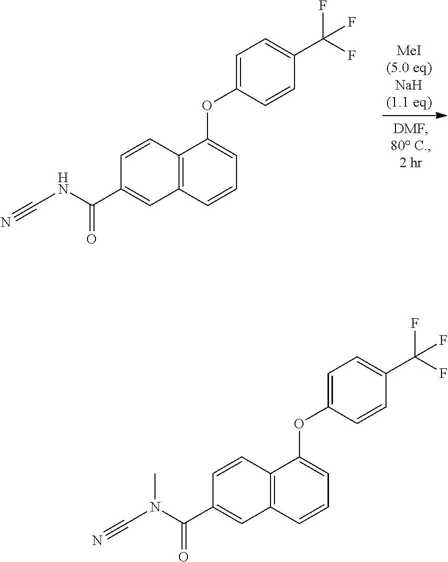 [SMILES: N#CNC(=O)c1ccc2c(Oc3ccc(C(F)(F)F)cc3)cccc2c1 ; CN(C#N)C(=O)c1ccc2c(Oc3ccc(C(F)(F)F)cc3)cccc2c1]

To a solution of N-cyano-5-[4-(trifluoromethyl)phenoxy]naphthalene-2-carboxamide (100 mg, 0.28 mmol, 1 eq) in DMF (2 mL) was added NaH (12.3 mg, 0.30 mmol, 60%, 1.1 eq) and Mel (199.1 mg, 1.40 mmol, 87.3 uL, 5 eq). The mixture was stirred at 80° C. for 2 hr. TLC (PE:EA=1:1) showed new spot was detected. The reaction mixture was diluted with H₂O (10 mL) and extracted with EA (10 mL*3). The combined organic phase was washed with brine (10 mL*2), dried with anhydrous Na₂SO₄, filtered and concentrated in vacuum. The residue was purified by flash silica gel chromatography (ISCO®; 4 g SepaFlash® Silica Flash Column, Eluent of 0-40% Ethyl acetate/Petroleum ether gradient @ 20 mL/min). The title compound (16.3 mg, 43.5 umol, 15.5% yield) was obtained as a white solid. LCMS (ESI): RT=1.006 min, mass calcd for C₂₀H₁₃F₃N₂O₂ 370.32 m/z found 371.1 [M+H]⁺. ¹H NMR (400 MHz, DMSO-d₆) δ 8.57 (s, 1H), 8.11 (br d, J=8.8 Hz, 1H), 8.03 (br d, J=8.3 Hz, 1H), 7.84 (br d, J=8.8 Hz, 1H), 7.79-7.66 (m, 3H), 7.41 (br d, J=7.5 Hz, 1H), 7.21 (br d, J=8.3 Hz, 2H), 3.37 (s, 3H).

### II. Biological Evaluation

### Example A1: YAP Reporter Assay

HEK293T cells stably transfected with 8×TBD luciferase reporter and pRLTK in 384-well plates were treated with the test compounds, starting from 3 μM (final concentration in assay plate), 1:3 dilution, and 10 points in quadruplicates. Post 24-hr incubation with compounds at 37° C. and 5% CO2, cells were lysed and 8×TBD-driven firefly luciferase and control TK-driven renilla luciferase activities were measured using Promega Dual-Luciferase Reporter Assay System.

Reagents: The reagents used for this study are: DMEM: Invitrogen #11960077, Dual-Glo Luciferase Assay System: Promega-E2980, Puromycin Dihydrochloride: Invitrogen-A1113803, 384-well plate: PerkinElmer-6007480, L-GLUTAMINE: Invitrogen-25030164, Hygromycin B: Invitrogen-10687010, and Penicillin-Streptomycin: Merk-TMS-AB2-C

Media: The media used for this assay were: Culture Medium: DMEM+1 ug/mL puromycin+200 ug/mL hygromycin (with 10% FBS+1 mM L-glutamine); and Assay Medium: DMEM (with 10% FBS+1 mM L-glutamine+1×P/S).

Cell Plating: The appropriate media was warmed at 37° C. by water bath: Culture Medium, Assay Medium, 1*D-PBS, 0.05% trypsin-EDTA. The cells were trypsinized after removing all media, then washed with 1*sterile D-PBS and then with 2 ml 0.05% trypsin-EDTA. The cells were then incubated at RT for one minute. Then 10 ml/75 cm2 flask Assay Medium was added to each flask. Using a 10 ml pipette, the cells were then gently resuspended in the media, until the clumps completely disappeared. The cells were then transferred into 50 ml centrifuge tubes and were centrifuged at 800 rpm for 5 mins. The medium was removed and the cells were resuspended with Assay Medium. An aliquot of cells was used to count the cell density (cells/ml). The cell suspension was then diluted with Assay Medium to a concentration of 6×104 cells/ml. 50 ul cells suspension was then plated to 384-well plate (PerkinElmer-6007480), 3×103 cells/well and the cells were incubated in an incubator at 37° C., 5% CO2.

Compound Treatment: In the afternoon (incubation of the plate with 3-4 hrs), the test compounds were added by Echo, starting from 3 uM (final concentration in the assay plate), 1:3 dilution, 10 points, quadruplicates. The plate was placed at 37° C., 5% CO2 incubator for 24 hrs.

Detection: The Dual-Glo Luciferase Reagent was prepared by transferring the contents of one bottle of Dual-Glo Luciferase Buffer to one bottle of Dual-Glo Luciferase Substrate to create the Dual-G Luciferase Reagent. Mixing was performed by inversion until the substrate was thoroughly dissolved. After mixing, the reagent was aliquoted into 15 ml tubes. In the afternoon (24 hrs post compound treatment), the DMEM+ medium in the 384 well plates were aspirated by Microplate Washer.

Measuring firefly luciferase activity: 20 ul Dual-Glo Luciferase Reagent was added to the 384-well plates. The plates were protected from light to prevent interference with the assay. The plates were shaken for mm followed centrifuging plates at 1000 rpm for 30 seconds. After waiting at least 10 minutes, the firefly luminescence was measured by Envision.

Measuring renilla luciferase activity: 20 ul Stop-Glo Reagent was added to the 384-well plates. The plates were shaken for m and then centrifuged at 1000 rpm for 30 seconds. After waiting at least 10 minutes, the renilla luminescence was measured by Envision.

Compound IC₅₀ and maximum inhibition on the firefly luciferase and renilla luciferase activities were reported separately. IC₅₀ for firefly luciferase activity are shown in Table 2.

TABLE 2

| | | Firefly |
|---|---|---|
| Compound | | Luciferase |
| # | Name | IC₅₀ (μM) |
| 1A | (3-aminoazetidin-1-yl)(5-(4- | A |
| | (trifluoromethyl)phenoxy)naphthalen-2-yl)methanone | |
| 1 | N-(1-(5-(4-(Trifluoromethyl)phenoxy)-2-naphthoyl)azetidin- | A |
| | 3-yl)acetamide | |
| 2 | Piperazin-1-yl(5-(4-(trifluoromethyl)phenoxy)naphthalen-2- | C |
| | yl)methanone | |
| 3 | (3-Aminopyrrolidin-1-yl)(5-(4- | B |
| | (trifluoromethyl)phenoxy)naphthalen-2-yl)methanone | |
| 4 | (4-Aminopiperidin-1-yl)(5-(4- | A |
| | (trifluoromethyl)phenoxy)naphthalen-2-yl)methanone | |
| 5 | (4-(Aminomethyl)piperidin-1-yl)(5-(4- | — |
| | (trifluoromethyl)phenoxy)naphthalen-2-yl)methanone | |
| 6 | (3-Aminopiperidin-1-yl)(5-(4- | — |
| | (trifluoromethyl)phenoxy)naphthalen-2-yl)methanone | |
| 7 | (3-(Aminomethyl)azetidin-1-yl)(5-(4- | A |
| | (trifluoromethyl)phenoxy)naphthalen-2-yl)methanone | |
| 8 | (3-(Aminomethyl)pyrrolidin-1-yl)(5-(4- | A |
| | (trifluoromethyl)phenoxy)naphthalen-2-yl)methanone | |
| 9 | (3-(Aminomethyl)piperidin-1-yl)(5-(4- | B |
| | (trifluoromethyl)phenoxy)naphthalen-2-yl)methanone | |
| 10 | N-((1-(5-(4-(trifluoromethyl)phenoxy)-2-naphthoyl)azetidin- | A |
| | 3-yl)methyl)acetamide | |
| 11 | (2,6-Diazaspiro[3.3]heptan-2-yl)(5-(4- | B |
| | (trifluoromethyl)phenoxy)naphthalen-2-yl)methanone | |
| 12 | (3-(Dimethylamino)azetidin-1-yl)(5-(4- | C |
| | (trifluoromethyl)phenoxy)naphthalen-2-yl)methanone | |
| 13 | (3-(Hydroxymethyl)azetidin-1-yl)(5-(4- | A |
| | (trifluoromethyl)phenoxy)naphthalen-2-yl)methanone | |
| 14 | (3-((Dimethylamino)methyl)azetidin-1-yl)(5-(4- | B |
| | (trifluoromethyl)phenoxy)naphthalen-2-yl)methanone | |
| 15 | (3-Hydroxyazetidin-1-yl)(5-(4- | A |
| | (trifluoromethyl)phenoxy)naphthalen-2-yl)methanone | |
| 16 | N-((1-(5-(4-(trifluoromethyl)phenoxy)-2-naphthoyl)azetidin- | A |
| | 3-yl)methyl)acetamide | |
| 17 | N-((1-(5-(4-(trifluoromethyl)phenoxy)-2-naphthoyl)azetidin- | A |
| | 3-yl)methyl)methanesulfonamide | |
| 18 | (6-Methyl-2,6-diazaspiro[3.3]heptan-2-yl)(5-(4- | B |
| | (trifluoromethyl)phenoxy)naphthalen-2-yl)methanone | |
| 19 | (6-Ethyl-2,6-diazaspiro[3.3]heptan-2-yl)(5-(4- | B |
| | (trifluoromethyl)phenoxy)naphthalen-2-yl)methanone | |
| 20 | (6-Isopropyl-2,6-diazaspiro[3.3]heptan-2-yl)(5-(4- | B |
| | (trifluoromethyl)phenoxy)naphthalen-2-yl)methanone | |
| 21 | (6-(2-Hydroxyethyl)-2,6-diazaspiro[3.3]heptan-2-yl)(5-(4- | B |
| | (trifluoromethyl)phenoxy)naphthalen-2-yl)methanone | |
| 22 | (6-(2-Methoxyethyl)-2,6-diazaspiro[3.3]heptan-2-yl)(5-(4- | B |
| | (trifluoromethyl)phenoxy)naphthalen-2-yl)methanone | |
| 23 | (6-(2-Fluoroethyl)-2,6-diazaspiro[3.3]heptan-2-yl)(5-(4- | B |
| | (trifluoromethyl)phenoxy)naphthalen-2-yl)methanone | |
| 24 | (6-(2,2-Difluoroethyl)-2,6-diazaspiro[3.3]heptan-2-yl)(5-(4- | B |
| | (trifluoromethyl)phenoxy)naphthalen-2-yl)methanone | |
| 25 | (3-((Methylamino)methyl)azetidin-1-yl)(5-(4- | B |
| | (trifluoromethyl)phenoxy)naphthalen-2-yl)methanone | |
| 26 | (3-(Methylamino)azetidin-1-yl)(5-(4- | B |
| | (trifluoromethyl)phenoxy)naphthalen-2-yl)methanone | |
| 27 | (3-(Ethylamino)azetidin-1-yl)(5-(4- | B |
| | (trifluoromethyl)phenoxy)naphthalen-2-yl)methanone | |
| 28 | (3-(Isopropylamino)azetidin-1-yl)(5-(4- | A |
| | (trifluoromethyl)phenoxy)naphthalen-2-yl)methanone | |
| 29 | (3-((2-Methoxyethyl)amino)azetidin-1-yl)(5-(4- | B |
| | (trifluoromethyl)phenoxy)naphthalen-2-yl)methanone | |
| 30 | (3-((2-Hydroxyethyl)amino)azetidin-1-yl)(5-(4- | A |
| | (trifluoromethyl)phenoxy)naphthalen-2-yl)methanone | |
| 31 | (3-((2-Fluoroethyl)amino)azetidin-1-yl)(5-(4- | B |
| | (trifluoromethyl)phenoxy)naphthalen-2-yl)methanone | |
| 32 | (3-((2,2-Difluoroethyl)amino)azetidin-1-yl)(5-(4- | B |
| | (trifluoromethyl)phenoxy)naphthalen-2-yl)methanone | |
| 33 | (3-((Ethylamino)methyl)azetidin-1-yl)(5-(4- | B |
| | (trifluoromethyl)phenoxy)naphthalen-2-yl)methanone | |
| 34 | (3-((Isopropylamino)methyl)azetidin-1-yl)(5-(4- | B |
| | (trifluoromethyl)phenoxy)naphthalen-2-yl)methanone | |
| 35 | (3-(((2-Methoxyethyl)amino)methyl)azetidin-1-yl)(5-(4- | B |
| | (trifluoromethyl)phenoxy)naphthalen-2-yl)methanone | |
| 36 | (3-(((2-Fluoroethyl)amino)methyl)azetidin-1-yl)(5-(4- | B |
| | (trifluoromethyl)phenoxy)naphthalen-2-yl)methanone | |
| 37 | (3-(((2-Hydroxyethyl)amino)methyl)azetidin-1-yl)(5-(4- | A |
| | (trifluoromethyl)phenoxy)naphthalen-2-yl)methanone | |
| 38 | (3-(((2,2-Fifluoroethyl)amino)methyl)azetidin-1-yl)(5-(4- | A |
| | (trifluoromethyl)phenoxy)naphthalen-2-yl)methanone | |
| 39 | (3-(Pyridin-2-ylamino)azetidin-1-yl)(5-(4- | B |
| | (trifluoromethyl)phenoxy)naphthalen-2-yl)methanone | |
| 40 | (3-((Pyridin-2-ylamino)methyl)azetidin-1-yl)(5-(4- | A |
| | (trifluoromethyl)phenoxy)naphthalen-2-yl)methanone | |
| 41 | (6-(Pyridin-2-yl)-2,6-diazaspiro[3.3]heptan-2-yl)(5-(4- | C |
| | (trifluoromethyl)phenoxy)naphthalen-2-yl)methanone | |
| 42 | N,N-dimethyl-5-(4-(trifluoromethyl)phenoxy)naphthalene-2- | B |
| | carboxamide | |
| 43 | N-cyano-N-methyl-5-(4-(trifluoromethyl)phenoxy)-2- | A |
| | naphthamide | |

Note:
Biochemical assay IC₅₀ data are designated within the following ranges:
A: ≤0.1 μM;
B: >0.1 μM to ≤1.0 μM;
C: >1.0 μM to ≤3 μM;
D: >3 μM ≤ 10 μM;

### Example A2: Tumor Suppression Assay

The procedures described herein for the tumor suppression assay is as described in PCT/US2013/043752 (WO 2013/188138). Mouse procedures are performed according to the guidelines of approved animal protocol and based on the methods. After the cells are grown to 90%>confluence, these cells are harvested by trypsinization, washed in phosphate-buffered saline (PBS), and resuspended in PBS supplemented with 50% Matrigel (BD Biosciences). An appropriate amount of cells is prepared for administration, such as 200 μL per injection site. Immuno-compromised mice are injected on the dorsolateral sites subcutaneously. Any one of the compounds described herein is formulated accordingly and is then administered at a suitable dose. Control mice received vehicle alone. The average tumor diameter (two perpendicular axes of the tumor are measured) are recorded. The data are expressed in tumor volume estimated by ([width]2×length/2). Paired, two-tailed Student's t-test is performed to access the statistical significance.

### Example A3: Cell Proliferation Assay

Cancer cell lines are plated in 384-well plates 24 h before drug treatment. Post incubation for various time periods with the test compounds, starting from 3 μM (final concentration in assay plate), 1:3 dilution, and 10 points in duplicates, the number of viable cells and proliferative cells are determined using CellTiter-Glo® Luminescent Cell Viability Assay Kit (Promega) and Click-iT EdU HCS Assay Kit (Invitrogen) according to the manufacturers' protocols. The IC₅₀ values and maximum % inhibition of the test compounds are calculated using the dose response curves.

The examples and embodiments described herein are for illustrative purposes only and various modifications or changes suggested to persons skilled in the art are to be included within the spirit and purview of this application and scope of the appended claims.

What is claimed is:

1. A compound of Formula (A), or a pharmaceutically acceptable salt or solvate thereof: 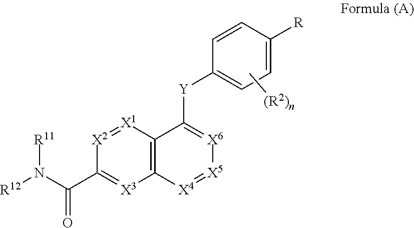
wherein,
X¹ is N or CR^{X1}; X² is N or CR^{X2}; X³ is N or CR^{X3}; X⁴ is N or CR^{X4}; X⁵ is N or CR^{X5}; X⁶ is N or CR^{X6};
Y is O, S, or NR³;
each R^{X1}, R^{X2}, R^{X3}, R^{X4}, R^{X5}, and R^{X6} is independently hydrogen, halogen, nitro, —OR³, —SR³, —CN, —C(═O)R³, —C(═O)NR³R⁴, —C(═O)OR³, —S(═O)R³, —S(═O)₂R³, —NR³R⁴, —NRS(═O)₂R³, —NR³C(═O)R³, —NR³C(═O)OR³, substituted or unsubstituted C₁-C₆alkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₇cycloalkyl, or substituted or unsubstituted C₂-C₆heterocycloalkyl;
R is halogen, nitro, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, or substituted or unsubstituted C₁-C₆fluoroalkyl;
R¹¹ and R¹² on the same nitrogen atom taken together with the nitrogen atom to which they are attached to form 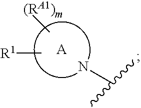
Ring A is substituted or unsubstituted N-containing heterocycloalkyl;
R¹ is hydrogen, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆ aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl;
m is 0, 1, 2, 3, 4, 5, or 6;
or each of R¹¹ and R¹² is independently —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R² is independently halogen, nitro, —CN, —OR³, —SR³, —S(═O)₂R³, —NR³R⁴, —C(═O)OR³, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R³ and R⁴ is independently hydrogen, —CN, substituted or unsubstituted C₁-C₆alkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or R³ and R⁴ on the same nitrogen atom taken together with the nitrogen atom to which they are attached to form a substituted or unsubstituted N-containing C₃-C₇ heterocycloalkyl; and
n is 0, 1, 2, 3, or 4.

2. The compound of claim 1, wherein the compound has the structure of Formula (I), or a pharmaceutically acceptable salt or solvate thereof: 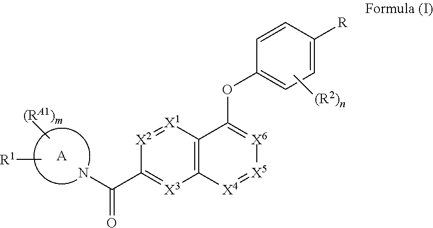
wherein,
Ring A is substituted or unsubstituted N-containing heterocycloalkyl;
X¹ is N or CR^{X1}; X² is N or CR^{X2}; X³ is N or CR^{X3}; X⁴ is N or CR^{X4}; X⁵ is N or CR^{X5}; X⁶ is N or CR^{X6};
each R^{X1}, R^{X2}, R^{X3}, R^{X4}, R^{X5}, and R^{X6} is independently hydrogen, halogen, nitro, —OR³, —SR³, —CN, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆ alkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₇cycloalkyl, or substituted or unsubstituted C₂-C₆heterocycloalkyl;
R is halogen, nitro, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, or substituted or unsubstituted C₁-C₆fluoroalkyl;
R¹ is hydrogen, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆ aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl;
each R² is independently halogen, nitro, —CN, —OR³, —SR³, —S(═O)₂R³, —NR³R⁴, —C(═O)OR³, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R³ and R⁴ is independently hydrogen, —CN, substituted or unsubstituted C₁-C₆alkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or R³ and R⁴ on the same nitrogen atom taken together with the nitrogen atom to which they are attached to form a substituted or unsubstituted N-containing C₃-C₇ heterocycloalkyl;
m is 0, 1, 2, 3, 4, 5, or 6; and
n is 0, 1, 2, 3, or 4.

3. The compound of claim 1 or claim 2, or a pharmaceutically acceptable salt or solvate thereof, wherein Ring A is substituted or unsubstituted monocyclic C₃-C₁₁heterocycloalkyl.

4. The compound of any one of claims 1-3, or a pharmaceutically acceptable salt or solvate thereof, wherein Ring A is substituted or unsubstituted monocyclic C₃-C₇heterocycloalkyl.

5. The compound of any one of claims 1-4, or a pharmaceutically acceptable salt or solvate thereof, wherein 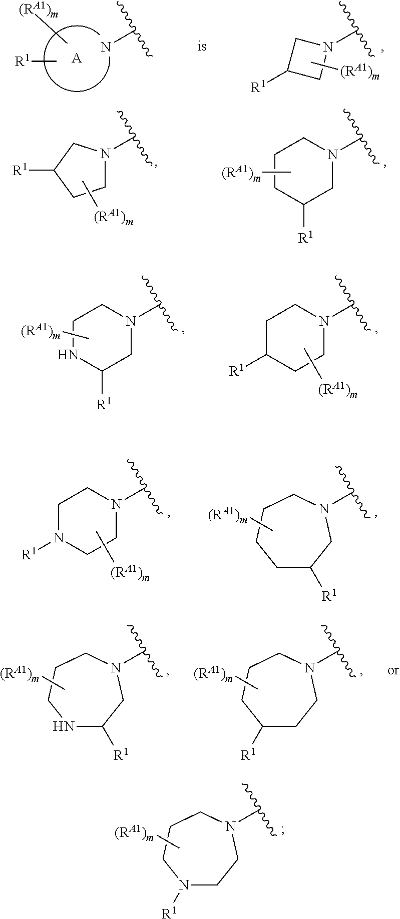
wherein each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl; R³ is hydrogen, substituted or unsubstituted C₁-C₄alkyl, or substituted or unsubstituted C₃-C₆cycloalkyl; and m is 0, 1, 2, 3, 4, 5, or 6.

6. The compound of any one of claims 1-5, or a pharmaceutically acceptable salt or solvate thereof, wherein

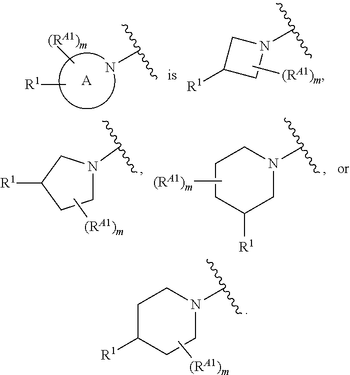

7. The compound of any one of claims 1-6, or a pharmaceutically acceptable salt or solvate thereof, wherein

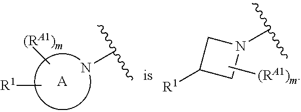

8. The compound of claim 1 or claim 2, or a pharmaceutically acceptable salt or solvate thereof, wherein Ring A is substituted or unsubstituted polycyclic C₃-C₁₆heterocycloalkyl.

9. The compound of any one of claims 1, 2, or 8, or a pharmaceutically acceptable salt or solvate thereof, wherein Ring A is substituted or unsubstituted fused- or spiro-C₄-C₁₂heterocycloalkyl.

10. The compound of any one of claims 1, 2, or 8-9, or a pharmaceutically acceptable salt or solvate thereof, wherein Ring A is substituted or unsubstituted fused C₄-C₁₂heterocycloalkyl.

11. The compound of any one of claims 1, 2, or 8-10, or a pharmaceutically acceptable salt or solvate thereof, wherein 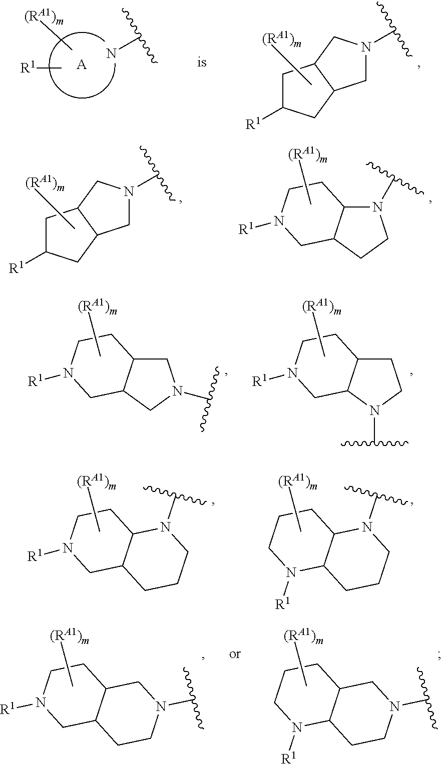
wherein each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl; R³ is hydrogen, substituted or unsubstituted C-C₄alkyl, or substituted or unsubstituted C₃-C₆cycloalkyl; and m is 0, 1, 2, 3, 4, 5, or 6.

12. The compound of any one of claims 1, 2, or 8-9, or a pharmaceutically acceptable salt or solvate thereof, wherein Ring A is substituted or unsubstituted spiro C₄-C₁₂heterocycloalkyl.

13. The compound of any one of claims 1, 2, 8-9, or 12, or a pharmaceutically acceptable salt or solvate thereof, wherein 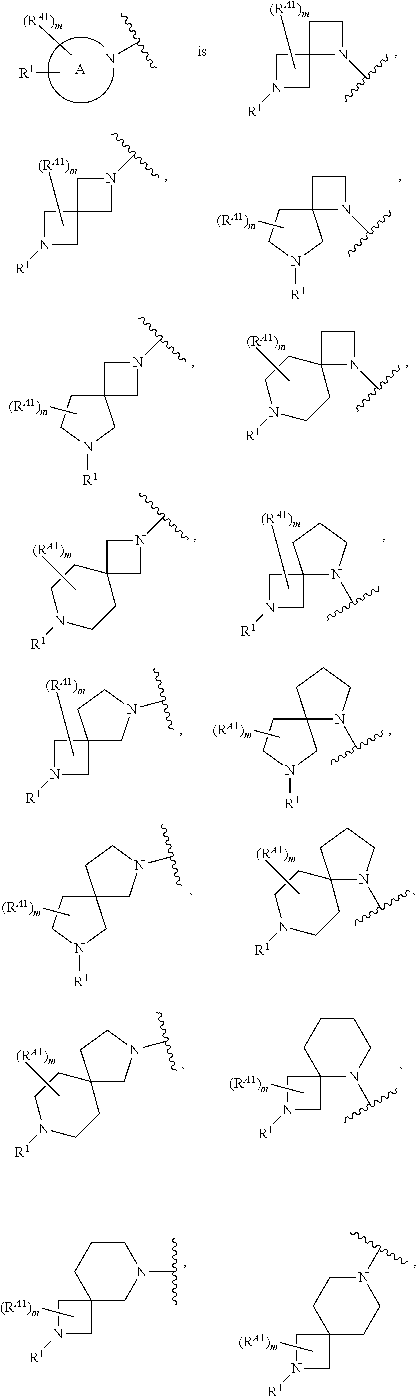 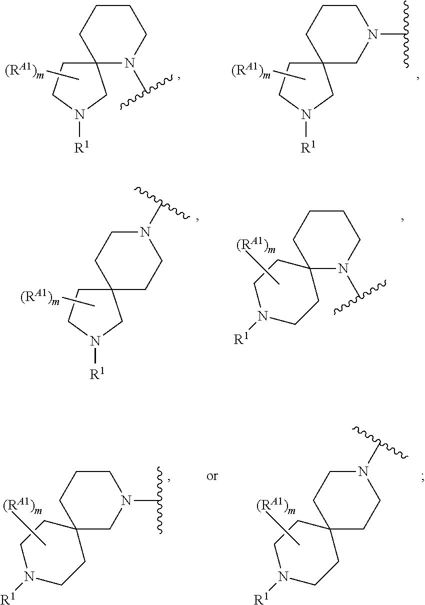
wherein each R^{A1} is independently F, —CN, —OR³, substituted or unsubstituted C₁-C₄alkyl, substituted or unsubstituted C₁-C₄heteroalkyl, substituted or unsubstituted C₃-C₆cycloalkyl, or substituted or unsubstituted C₂-C₅heterocycloalkyl; R³ is hydrogen, substituted or unsubstituted C₁-C₄alkyl, or substituted or unsubstituted C₃-C₆cycloalkyl; and m is 0, 1, 2, 3, 4, 5, or 6.

14. The compound of claim 1 or claim 2, or a pharmaceutically acceptable salt or solvate thereof, wherein:

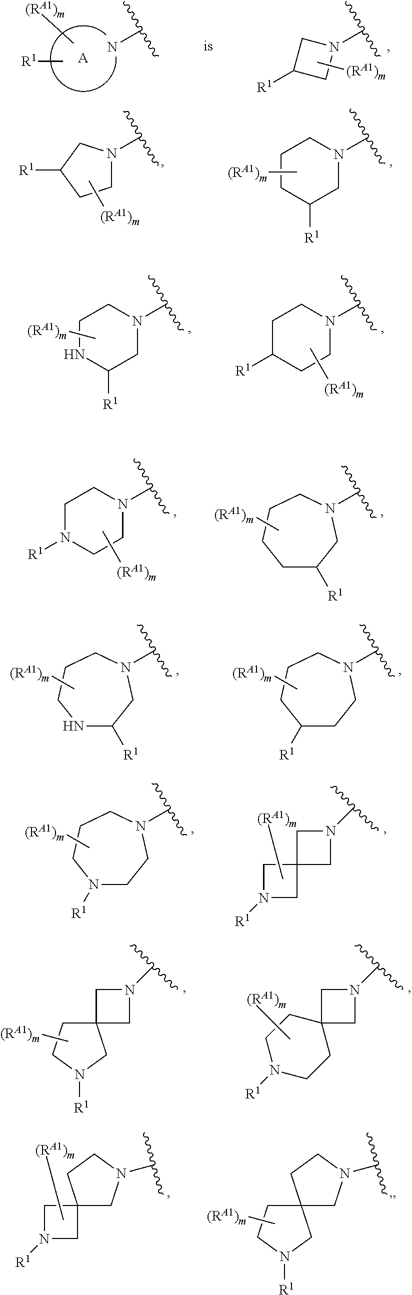

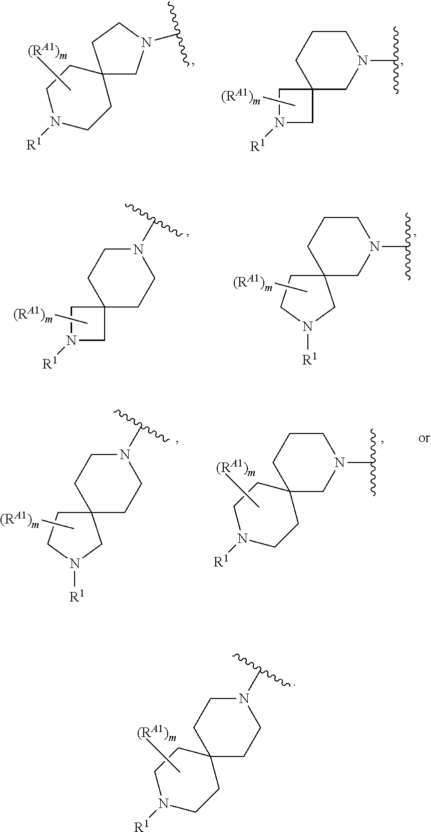

15. The compound of claim 1 or claim 2, or a pharmaceutically acceptable salt or solvate thereof, wherein:

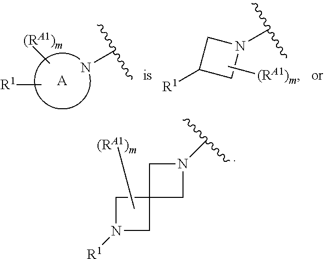

16. The compound of any one of claims 1-15, or a pharmaceutically acceptable salt or solvate thereof, wherein:
each R^{A1} is independently F, —CN, —OR³, or substituted or unsubstituted C₁-C₄alkyl; and R³ is hydrogen, substituted or unsubstituted C₁-C₄alkyl, or substituted or unsubstituted C₃-C₆cycloalkyl.

17. The compound of any one of claims 1-15, or a pharmaceutically acceptable salt or solvate thereof, wherein each R^{A1} is independently F, —OH, —CH₃, or —OCH₃.

18. The compound of any one of claims 1-17, or a pharmaceutically acceptable salt or solvate thereof, wherein m is 1 or 2.

19. The compound of any one of claims 1-15, or a pharmaceutically acceptable salt or solvate thereof, wherein m is 0.

20. The compound of any one of claims 1-19, or a pharmaceutically acceptable salt or solvate thereof, wherein R¹ is —OR³, —SR³, —C(═O)R³, —C(═O)NR³R⁴, —C(═O)OR³, —NR³R⁴, substituted or unsubstituted C₁-C₆ alkyl, or substituted or unsubstituted C₁-C₆ aminoalkyl; and each R³ and R⁴ is independently hydrogen, substituted or unsubstituted C₁-C₄alkyl, or substituted or unsubstituted C₃-C₆cycloalkyl.

21. The compound of any one of claims 1-20, or a pharmaceutically acceptable salt or solvate thereof, wherein R¹ is substituted or unsubstituted C₁-C₆ alkyl.

22. The compound of claim 21, or a pharmaceutically acceptable salt or solvate thereof, wherein R¹ is C₁-C₄ alkyl substituted with —OR³ or —NR³R⁴.

23. The compound of claim 22, or a pharmaceutically acceptable salt or solvate thereof, wherein each R³ and R⁴ is independently hydrogen or C₁-C₄ alkyl.

24. The compound of claim 22 or 23, or a pharmaceutically acceptable salt or solvate thereof, wherein R¹ is C₁-C₄ alkyl substituted with —OH or —NH₂.

25. The compound of any one of claims 22-24, or a pharmaceutically acceptable salt or solvate thereof, wherein R¹ is —CH₂OH, —CH₂CH₂OH, —CH₂NH₂, or —CH₂CH₂NH₂.

26. The compound of any one of claims 1-20, or a pharmaceutically acceptable salt or solvate thereof, wherein R¹ is —OR³ or —NR³R⁴.

27. The compound of claim 26, or a pharmaceutically acceptable salt or solvate thereof, wherein each R³ and R⁴ is independently hydrogen or C₁-C₄ alkyl.

28. The compound or a pharmaceutically acceptable salt or solvate thereof of claim 26 or claim 27, wherein R¹ is —NH₂ or —OH.

29. A compound of Formula (II), or a pharmaceutically acceptable salt or solvate thereof: 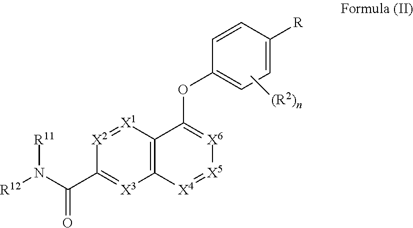
wherein,
X¹ is N or CR^{X1}; X² is N or CR^{X2}; X³ is N or CR^{X3}; X⁴ is N or CR^{X4}; X⁵ is N or CR^{X5}; X⁶ is N or CR^{X6};
each R^{X1}, R^{X2}, R^{X3}, R^{X4}, R^{X5}, and R^{X6} is independently hydrogen, halogen, nitro, —OR³, —SR³, —CN, —C(═O)R³, —C(═O)NR³R⁴, —C(═O)OR³, —S(═O)R³, —S(═O)₂R³, —NR³R⁴, —NRS(═O)₂R³, —NR³C(═O)R³, —NR³C(═O)OR³, substituted or unsubstituted C₁-C₆alkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₇cycloalkyl, or substituted or unsubstituted C₂-C₆heterocycloalkyl;
R is halogen, nitro, —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, or substituted or unsubstituted C₁-C₆fluoroalkyl;
each R¹¹ and R¹² is independently —CN, —OR³, —SR³, —C(═O)R³, —C(═O)NR³R⁴, —C(═O)OR³, —S(═O)R³, —S(═O)₂R³, —NR³R⁴, —NR³S(═O)₂R³, —NR³C(═O)R³, —NR³C(═O)OR³, substituted or unsubstituted C₁-C₆alkyl, substituted or unsubstituted C₁-C₆aminoalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R² is independently halogen, nitro, —CN, —OR³, —SR³, —S(═O)₂R³, —NR³R⁴, —C(═O)OR³, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R³ and R⁴ is independently hydrogen, —CN, substituted or unsubstituted C₁-C₆alkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or R³ and R⁴ on the same nitrogen atom taken together with the nitrogen atom to which they are attached to form a substituted or unsubstituted N-containing C₃-C₇ heterocycloalkyl; and
n is 0, 1, 2, 3, or 4.

30. The compound of claim 1 or claim 29, or a pharmaceutically acceptable salt or solvate thereof, wherein each R¹¹ and R¹² is independently —CN, —OR³, —SR³, —C(═O)R³, —C(═O)NR³R⁴, —C(═O)OR³, —S(═O)R³, —S(═O)₂R³, —NR³R⁴, substituted or unsubstituted C₁-C₆alkyl, substituted or unsubstituted C₁-C₆aminoalkyl, or substituted or unsubstituted C₃-C₁₀cycloalkyl; and each R³ and R⁴ is independently hydrogen, substituted or unsubstituted C₁-C₄alkyl, or substituted or unsubstituted C₃-C₆cycloalkyl.

31. The compound of any one of claims 1, 29, or 30, or a pharmaceutically acceptable salt or solvate thereof, wherein
each R¹¹ and R¹² is independently —CN, —OR³, —S(═O)₂R³, —NR³R⁴, substituted or unsubstituted C₁-C₆alkyl, or substituted or unsubstituted C₁-C₆aminoalkyl.

32. The compound of any one of claim 1 or 29-31, or a pharmaceutically acceptable salt or solvate thereof, wherein
each R¹¹ and R¹² is independently —CN or substituted or unsubstituted C₁-C₆alkyl.

33. The compound of any one of claim 1 or 29-32, or a pharmaceutically acceptable salt or solvate thereof, wherein
each R¹¹ and R¹² is independently —CN, —CH₃, —CH₂CH₃, or —CH₂CH₂CH₃.

34. The compound of any one of claims 1-33, or a pharmaceutically acceptable salt or solvate thereof, wherein X¹ is CR^{X1}; X¹ is CR^{X2}; and X³ is CR^{X3}.

35. The compound of any one of claims 1-33, or a pharmaceutically acceptable salt or solvate thereof, wherein X¹ is N; X¹ is CR^{X2}; and X³ is CR^{X3}.

36. The compound of any one of claims 1-33, or a pharmaceutically acceptable salt or solvate thereof, wherein X¹ is CR^{X1}; X¹ is CR^{X2}; and X³ is N.

37. The compound of any one of claims 1-36 or a pharmaceutically acceptable salt or solvate thereof, wherein X⁴ is CR^{X4}; X⁵ is CR^{X5}; and X⁶ is CR^{X6}.

38. The compound of any one of claims 1-36, or a pharmaceutically acceptable salt or solvate thereof, wherein X⁴ is CR^{X4}; X⁵ is CR^{X5}; and X⁶ is N.

39. The compound of any one of claims 1-38, or a pharmaceutically acceptable salt or solvate thereof, wherein
each R^{X1}, R^{X2}, R^{X3}, R^{X4}, R^{X5}, and R^{X6}, when present, is independently hydrogen, halogen, —OR³, —SR³, —CN, —S(═O)R³, —S(═O)₂R³, —NR³R⁴, —NR³S(═O)₂R³, —NR³C(═O)R³, substituted or unsubstituted C₁-C₆alkyl, substituted or unsubstituted C₁-C₆fluoroalkyl, substituted or unsubstituted C₂-C₄alkenyl, substituted or unsubstituted C₂-C₄alkynyl, or substituted or unsubstituted C₁-C₆heteroalkyl; and
each R³ and R⁴ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, or substituted or unsubstituted C₂-C₁₀heterocycloalkyl; or R³ and R⁴ are taken together with the nitrogen atom to which they are attached to form a substituted or unsubstituted C₃-C₇ heterocycloalkyl.

40. The compound of any one of claims 1-38, or a pharmaceutically acceptable salt or solvate thereof, wherein each R^{X1}, R^{X2}, R^{X3}, R^{X4}, R^{X5}, and R^{X6}, when present, is independently hydrogen, F, Cl, Br, I, —CH₃, —CH₂CH₃, cyclopropyl, —C≡CH, —OH, —OCH₃, —OCH₂CH₃, —OCF₃, —SCH₃, cyclopropyloxy, —NH₂, —NHC(═O)CH₃, —N(CH₃)C(═O)CH₃, —NHS(═O)₂CH₃, —N(CH₃)S(═O)₂CH₃, —S(═O)CH₃, or —S(═O)₂CH₃.

41. The compound of any one of claims 1-38, or a pharmaceutically acceptable salt or solvate thereof, wherein each R^{X1}, R^{X2}, R^{X3}, R^{X4}, R^{X5}, and R^{X6}, when present, is independently hydrogen, F, Cl, Br, —CH₃, —OH, —OCH₃, or —OCF₃.

42. The compound of any one of claims 1-38, or a pharmaceutically acceptable salt or solvate thereof, wherein each R^{X1}, R^{X2}, R^{X3}, R^{X4}, R^{X5}, and R^{X6}, when present, is independently hydrogen, F, or —OCH₃.

43. The compound of any one of claims 1-38, or a pharmaceutically acceptable salt or solvate thereof, wherein each R^{X1}, R^{X2}, R^{X3}, R^{X4}, R^{X5}, and R^{X6}, when present, is hydrogen.

44. The compound of any one of claims 1-41, or a pharmaceutically acceptable salt or solvate thereof, wherein:
R is halogen, nitro, —CN, —OR³, —C(═O)R³, —C(═O)NR³R⁴, —C(═O)OR³, —S(═O)R³, —S(═O)₂R³, —NR³S(═O)₂R³, —NR³C(═O)R³, —NR³C(═O)OR³, or substituted or unsubstituted C₁-C₆fluoroalkyl; and
each R³ and R⁴ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, or substituted or unsubstituted C₂-C₁₀heterocycloalkyl; or R³ and R⁴ are taken together with the nitrogen atom to which they are attached to form a substituted or unsubstituted C₃-C₇ heterocycloalkyl.

45. The compound of any one of claims 1-43, or a pharmaceutically acceptable salt or solvate thereof, wherein R is F, Cl, Br, I, nitro, —CN, —OCH₂F, —OCHF₂, —OCF₃, —C(═O)CH₃, —C(═O)OCH₃—C(═O)NH₂, —C(═O)NHCH₃, —C(═O)N(CH₃)₂, —S(═O)CH₃, —S(═O)₂CH₃, —NHS(═O)₂CH₃, —N(CH₃)S(═O)₂CH₃, —NHC(═O)CH₃, —N(CH₃)C(═O)CH₃, —NHC(═O)OCH₃, —N(CH₃)C(═O)OCH₃, —CH₂F, —CHF₂, or —CF₃.

46. The compound of any one of claims 1-43, or a pharmaceutically acceptable salt or solvate thereof, wherein R is F, Cl, —CN, —OCF₃, —CHF₂, or —CF₃.

47. The compound of any one of claims 1-43, or a pharmaceutically acceptable salt or solvate thereof, wherein R is F, Cl, —OCF₃, —CHF₂, or —CF₃.

48. The compound of any one of claims 1-43, or a pharmaceutically acceptable salt or solvate thereof, wherein R is —CF₃.

49. The compound of any one of claims 1-48, or a pharmaceutically acceptable salt or solvate thereof, wherein
each R² is independently halogen, nitro, —CN, —OR³, or substituted or unsubstituted C₁-C₆alkyl; and
each R³ is independently hydrogen, substituted or unsubstituted C₁-C₆alkyl, or substituted or unsubstituted C₁-C₆fluoroalkyl.

50. The compound of any one of claims 1-48, or a pharmaceutically acceptable salt or solvate thereof, wherein each R² is independently F, Cl, —CN, —OCH₃, —OCF₃, or —CF₃.

51. The compound of any one of claims 1-48, or a pharmaceutically acceptable salt or solvate thereof, wherein each R² is independently F, Cl, —OCF₃, or —CF₃.

52. The compound of any one of claims 1-48, or a pharmaceutically acceptable salt or solvate thereof, wherein each R² is independently F or Cl.

53. A compound that has one of the following structures: 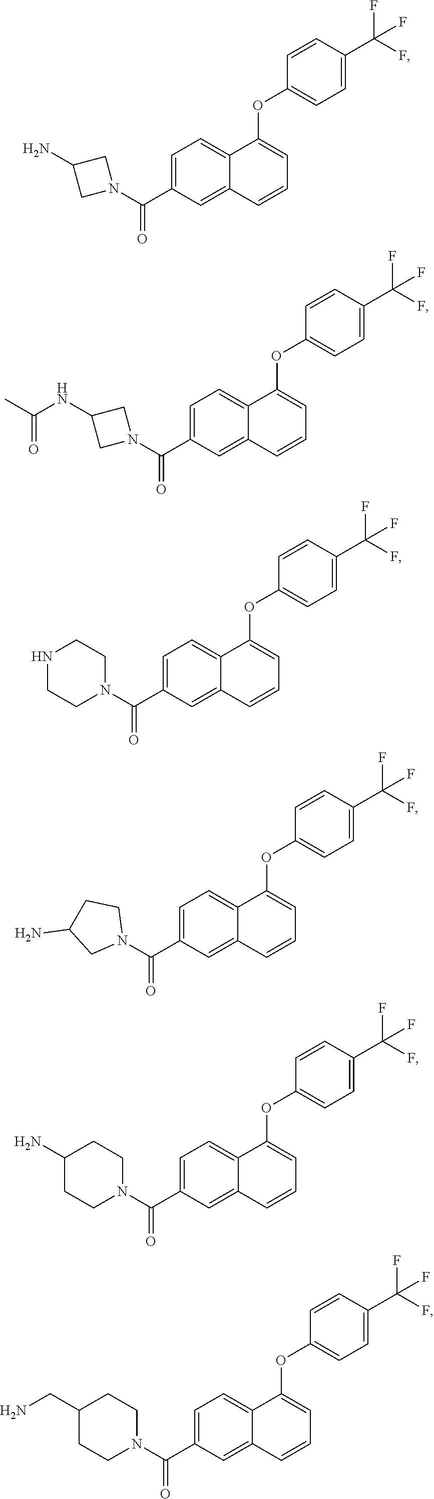 [SMILES: NC1CN(C(=O)c2ccc3c(Oc4ccc(C(F)(F)F)cc4)cccc3c2)C1 ; CC(=O)NC1CN(C(=O)c2ccc3c(Oc4ccc(C(F)(F)F)cc4)cccc3c2)C1 ; O=C(c1ccc2c(Oc3ccc(C(F)(F)F)cc3)cccc2c1)N1CCNCC1 ; NC1CCN(C(=O)c2ccc3c(Oc4ccc(C(F)(F)F)cc4)cccc3c2)C1 ; NC1CCN(C(=O)c2ccc3c(Oc4ccc(C(F)(F)F)cc4)cccc3c2)CC1 ; NCC1CCN(C(=O)c2ccc3c(Oc4ccc(C(F)(F)F)cc4)cccc3c2)CC1] 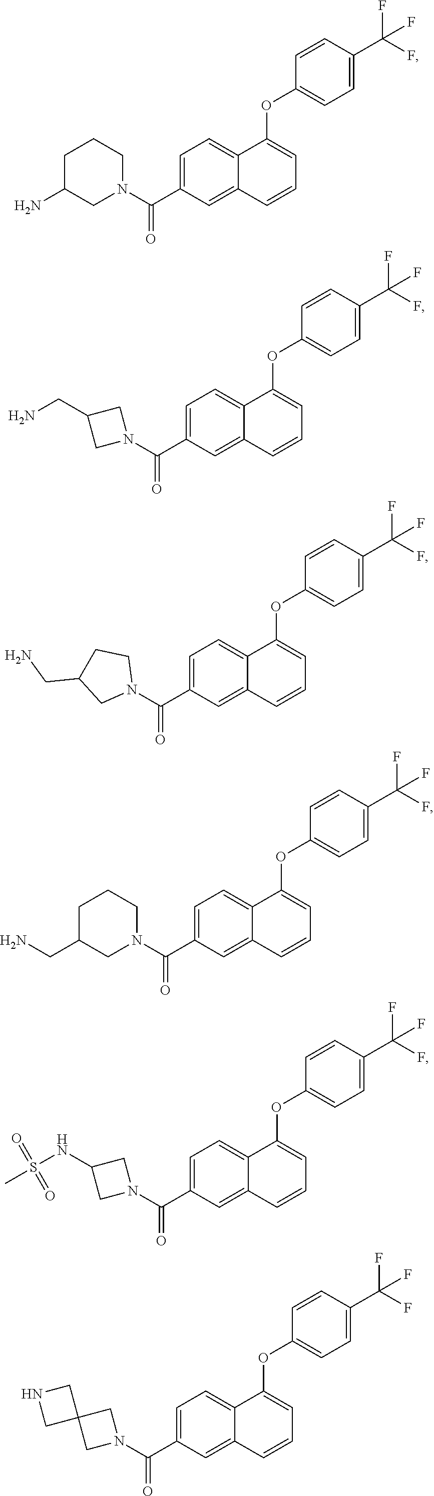 [SMILES: NC1CCCN(C(=O)c2ccc3c(Oc4ccc(C(F)(F)F)cc4)cccc3c2)C1 ; NCC1CN(C(=O)c2ccc3c(Oc4ccc(C(F)(F)F)cc4)cccc3c2)C1 ; NCC1CCN(C(=O)c2ccc3c(Oc4ccc(C(F)(F)F)cc4)cccc3c2)C1 ; NCC1CCCN(C(=O)c2ccc3c(Oc4ccc(C(F)(F)F)cc4)cccc3c2)C1 ; CS(=O)(=O)NC1CN(C(=O)c2ccc3c(Oc4ccc(C(F)(F)F)cc4)cccc3c2)C1 ; O=C(c1ccc2c(Oc3ccc(C(F)(F)F)cc3)cccc2c1)N1CC2(CNC2)C1] 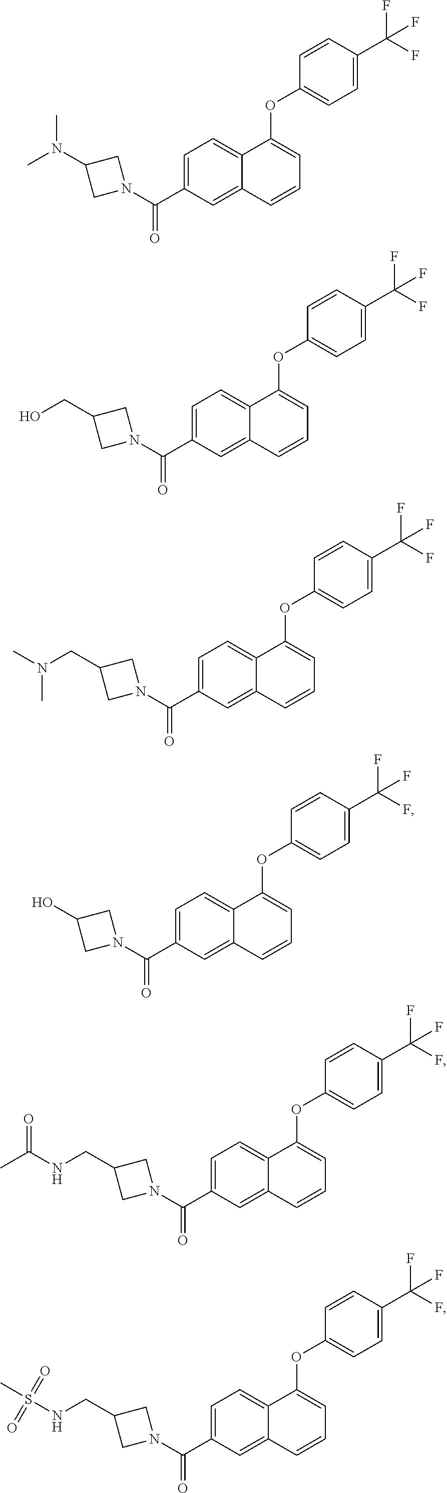 [SMILES: CN(C)C1CN(C(=O)c2ccc3c(Oc4ccc(C(F)(F)F)cc4)cccc3c2)C1 ; O=C(c1ccc2c(Oc3ccc(C(F)(F)F)cc3)cccc2c1)N1CC(CO)C1 ; CN(C)CC1CN(C(=O)c2ccc3c(Oc4ccc(C(F)(F)F)cc4)cccc3c2)C1 ; O=C(c1ccc2c(Oc3ccc(C(F)(F)F)cc3)cccc2c1)N1CC(O)C1 ; CC(=O)NCC1CN(C(=O)c2ccc3c(Oc4ccc(C(F)(F)F)cc4)cccc3c2)C1 ; CS(=O)(=O)NCC1CN(C(=O)c2ccc3c(Oc4ccc(C(F)(F)F)cc4)cccc3c2)C1] 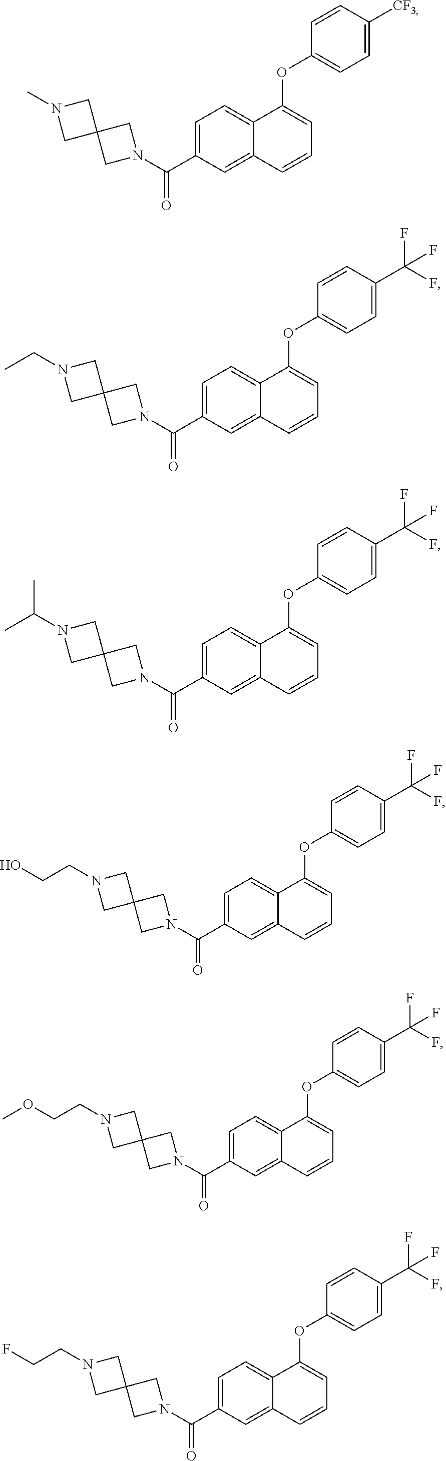 [SMILES: CN1CC2(C1)CN(C(=O)c1ccc3c(Oc4ccc(C(F)(F)F)cc4)cccc3c1)C2 ; CCN1CC2(C1)CN(C(=O)c1ccc3c(Oc4ccc(C(F)(F)F)cc4)cccc3c1)C2 ; CC(C)N1CC2(CN(C(=O)c3ccc4c(Oc5ccc(C(F)(F)F)cc5)cccc4c3)C2)C1 ; O=C(c1ccc2c(Oc3ccc(C(F)(F)F)cc3)cccc2c1)N1CC2(CN(CCO)C2)C1 ; COCCN1CC2(C1)CN(C(=O)c1ccc3c(Oc4ccc(C(F)(F)F)cc4)cccc3c1)C2 ; O=C(c1ccc2c(Oc3ccc(C(F)(F)F)cc3)cccc2c1)N1CC2(CN(CCF)C2)C1] 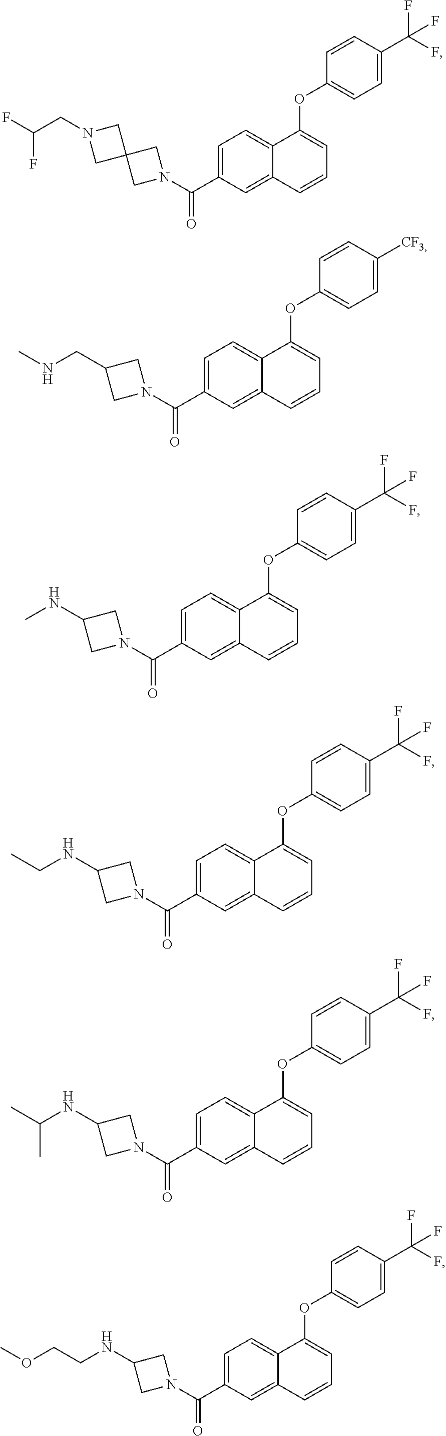 [SMILES: O=C(c1ccc2c(Oc3ccc(C(F)(F)F)cc3)cccc2c1)N1CC2(CN(CC(F)F)C2)C1 ; CNCC1CN(C(=O)c2ccc3c(Oc4ccc(C(F)(F)F)cc4)cccc3c2)C1 ; CNC1CN(C(=O)c2ccc3c(Oc4ccc(C(F)(F)F)cc4)cccc3c2)C1 ; CCNC1CN(C(=O)c2ccc3c(Oc4ccc(C(F)(F)F)cc4)cccc3c2)C1 ; CC(C)NC1CN(C(=O)c2ccc3c(Oc4ccc(C(F)(F)F)cc4)cccc3c2)C1 ; COCCNC1CN(C(=O)c2ccc3c(Oc4ccc(C(F)(F)F)cc4)cccc3c2)C1] 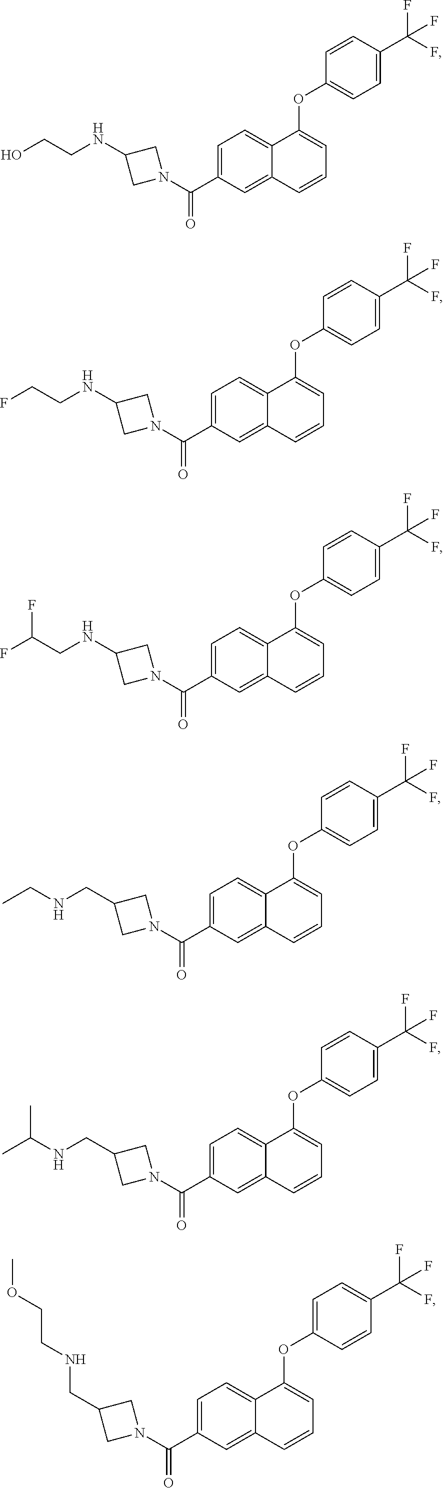 [SMILES: O=C(c1ccc2c(Oc3ccc(C(F)(F)F)cc3)cccc2c1)N1CC(NCCO)C1 ; O=C(c1ccc2c(Oc3ccc(C(F)(F)F)cc3)cccc2c1)N1CC(NCCF)C1 ; O=C(c1ccc2c(Oc3ccc(C(F)(F)F)cc3)cccc2c1)N1CC(NCC(F)F)C1 ; CCNCC1CN(C(=O)c2ccc3c(Oc4ccc(C(F)(F)F)cc4)cccc3c2)C1 ; CC(C)NCC1CN(C(=O)c2ccc3c(Oc4ccc(C(F)(F)F)cc4)cccc3c2)C1 ; COCCNCC1CN(C(=O)c2ccc3c(Oc4ccc(C(F)(F)F)cc4)cccc3c2)C1] 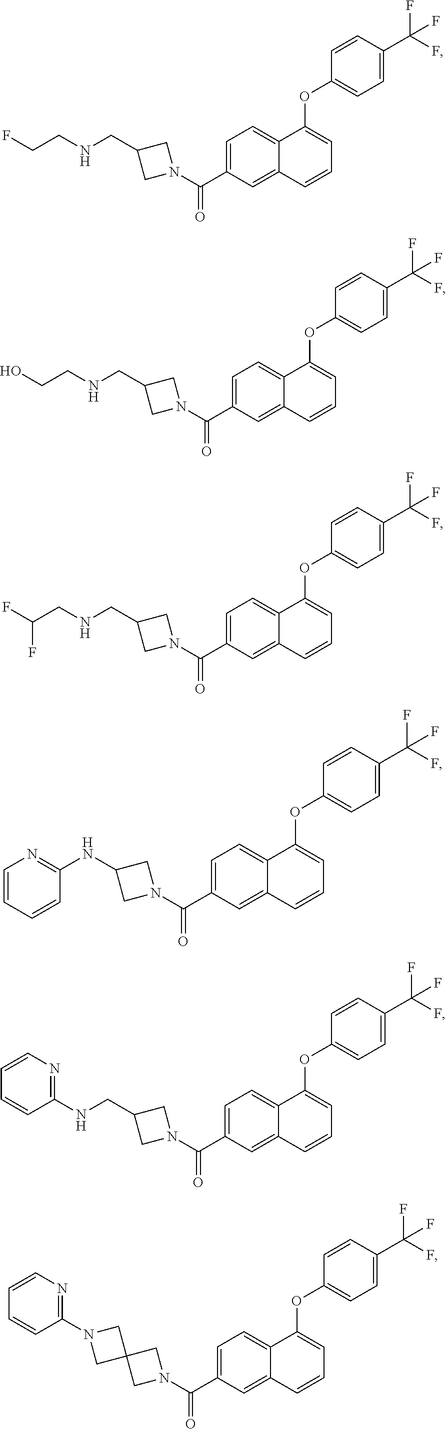 [SMILES: O=C(c1ccc2c(Oc3ccc(C(F)(F)F)cc3)cccc2c1)N1CC(CNCCF)C1 ; O=C(c1ccc2c(Oc3ccc(C(F)(F)F)cc3)cccc2c1)N1CC(CNCCO)C1 ; O=C(c1ccc2c(Oc3ccc(C(F)(F)F)cc3)cccc2c1)N1CC(CNCC(F)F)C1 ; O=C(c1ccc2c(Oc3ccc(C(F)(F)F)cc3)cccc2c1)N1CC(Nc2ccccn2)C1 ; O=C(c1ccc2c(Oc3ccc(C(F)(F)F)cc3)cccc2c1)N1CC(CNc2ccccn2)C1 ; O=C(c1ccc2c(Oc3ccc(C(F)(F)F)cc3)cccc2c1)N1CC2(C1)CN(c1ccccn1)C2] 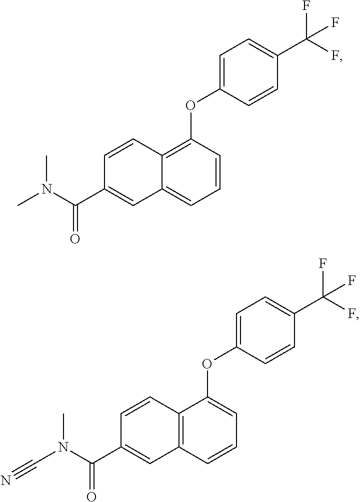 [SMILES: CN(C)C(=O)c1ccc2c(Oc3ccc(C(F)(F)F)cc3)cccc2c1 ; CN(C#N)C(=O)c1ccc2c(Oc3ccc(C(F)(F)F)cc3)cccc2c1]
or a pharmaceutically acceptable salt or solvate thereof.

54. A pharmaceutical composition comprising a pharmaceutically acceptable excipient and a compound of any one of claims 1-53, or a pharmaceutically acceptable salt or solvate thereof.

55. A method of inhibiting one or more of proteins encompassed by, or related to, the Hippo pathway in a subject, comprising administering to a subject a compound of any one of claims 1-53, or a pharmaceutically acceptable salt or solvate thereof.

56. A method of inhibiting transcriptional coactivator with PDZ binding motif/Yes-associated protein transcriptional coactivator (TAZ/YAP) in a subject comprising administering to a subject a compound of any one of claims 1-53, or a pharmaceutically acceptable salt or solvate thereof.

57. The method of claim 55 or claim 56, wherein the subject has cancer, polycystic kidney disease or liver fibrosis.

58. The method of claim 57, wherein the cancer is selected from mesothelioma, hepatocellular carcinoma, meningioma, malignant peripheral nerve sheath tumor, Schwannoma, lung cancer, bladder carcinoma, cutaneous neurofibromas, prostate cancer, pancreatic cancer, glioblastoma, endometrial adenosquamous carcinoma, anaplastic thyroid carcinoma, gastric adenocarcinoma, esophageal adenocarcinoma, ovarian cancer, ovarian serous adenocarcinoma, melanoma, and breast cancer.

59. A method of treating cancer in a subject in need thereof comprising administering to the subject in need thereof a therapeutically effective amount of a compound of any one of claims 1-53, or a pharmaceutically acceptable salt or solvate thereof.

60. The method of claim 59, wherein the cancer is selected from mesothelioma, hepatocellular carcinoma, meningioma, malignant peripheral nerve sheath tumor, Schwannoma, lung cancer, bladder carcinoma, cutaneous neurofibromas, prostate cancer, pancreatic cancer, glioblastoma, endometrial adenosquamous carcinoma, anaplastic thyroid carcinoma, gastric adenocarcinoma, esophageal adenocarcinoma, ovarian cancer, ovarian serous adenocarcinoma, melanoma, and breast cancer.

61. A method of treating polycystic kidney disease or liver fibrosis in a subject in need thereof comprising administering to the subject in need thereof a therapeutically effective amount of a compound of any one of claims 1-53, or a pharmaceutically acceptable salt or solvate thereof.
